(19) United States
(12) Patent Application Publication
(10) Pub. No.: US 2010/0119454 A1
(43) Pub. Date: May 13, 2010

(54) Use of the conserved Drosophila NPFR1 system for uncovering interacting genes and pathways important in nociception and stress response

(76) Inventors: Ping Shen, Athens, GA (US); Jie Xu, Athens, GA (US); Mo Li, Athens, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/590,159

(22) Filed: Nov. 3, 2009

Related U.S. Application Data
(60) Provisional application No. 61/110,699, filed on Nov. 3, 2008.

(51) Int. Cl. A61K 49/00; A01K 67/00; C12Q 1/02; C12N 5/00

(52) U.S. Cl. 424/9.2; 800/3; 800/13; 435/29; 435/325

(57) ABSTRACT

The present disclosure provides methods of identifying compositions capable of inhibiting responses to stressors in an organism, as well as recombinant cells and organisms useful in identifying compositions capable of inhibiting responses to stressors in an organism.

### CROSS-REFERENCE TO RELATED APPLICATION

This application claims priority to U.S. provisional application entitled, “USE OF THE CONSERVED DROSOPHILA NPFR1 SYSTEM FOR UNCOVERING INTERACTING GENES AND PATHWAYS IMPORTANT IN NOCICEPTION AND STRESS RESPONSE,” having Ser. No. 61/110,699, filed on Nov. 3, 2008, which is entirely incorporated herein by reference.

### STATEMENT REGARDING FEDERALLY SPONSORED RESEARCH OR DEVELOPMENT

This invention was made with Government support under Contract/Grant Nos. AA014348 and DK058348, awarded by the National Institutes of Health (NIH). The Government has certain rights in this invention.

### BACKGROUND

Sensory systems define an organism's perception of its environment and play a key role in behavioral development and adaptation. Many organisms display modified behavioral responses to particular sensory stimuli and such behavioral responses may change or evolve in an age-dependent manner as the organism develops. Much is still unknown regarding the chemical pathways and regulatory mechanisms underlying biological responses to sensory stimuli as well as developmentally programmed modifications in such responses.

Pain and other sensations represent subjective experiences related to an organism's perception of inputs to the central nervous system by a specific class of sensory receptors. Organisms have various sensory receptors designed to respond to many different sensory inputs. Some receptors are activated by multiple stimuli, while others are specific. Receptors for what would be considered adverse or unpleasant stimuli or “stressors” are known as nociceptors. Such nociceptors react in response to various stimuli such as noxious mechanical, thermal, and chemical stimuli. However, in some organisms, the response to certain stimuli will elicit an avoidance response, while eliciting no response or even a positive response during other stages of development, thereby indicating a developmental change in the activation of the specific nociceptors to the stimuli.

Neuropeptide Y (NPY) family peptides are conserved structurally and functionally among diverse species including flies and humans. Recent human studies suggest that individuals with haplotypes associated with low NPY expression display diminished resiliency to stress and pain, and higher NPY levels may help prevent posttraumatic stress disorders. In Drosophila, neuropeptide F (NPF, the sole fly homolog of human NPY) displays parallel activities to NPY including promotion of resilience of foraging animals to diverse stressors and suppression of certain behaviors. However, it remains unclear how NPY family peptides modulate physical and emotional responses to various stressors and whether other compounds could be identified to mimic or modulate such activities.

### SUMMARY

Briefly described, embodiments of the present disclosure provide for methods of identifying compositions capable of inhibiting responses to stressors in an organism, and recombinant cells and organisms useful in identifying compositions capable of inhibiting responses to stressors in an organism.

One exemplary method of the present disclosure, among others, includes identifying a composition capable of inhibiting a response to a stressor by exposing a Drosophila melanogaster organism to a medium containing a compound that elicits an avoidance response in a wild-type Drosophila organism, where the Drosophila organism exhibits an avoidance response to the medium, and then contacting the Drosophila organism with a test compound. A reduction in the avoidance response to the medium in the presence of the test compound as compared to in the absence of the test compound indicates that the test compound modulates response to a stressor in a higher organism.

Another exemplary method of identifying a composition capable of modulating a response to a stressor, includes providing a recombinant Drosophila melanogaster organism having a heterologous transient receptor potential (TRP) ion-channel polypeptide from a different organism, where the TRP ion-channel polypeptide is responsive to a particular stressor; exposing the larva to the stressor, where the Drosophila larva exhibits an avoidance response to the stressor; and contacting the larva with a test compound. A reduction in the avoidance response to the stressor in the presence of the test compound as compared to in the absence of the test compound indicates that the test compound modulates response to a stressor in a higher organism. The disclosure also includes the recombinant Drosophila organisms described in the method.

Embodiments of the present disclosure also provide, among others, a method of identifying a composition capable of inhibiting a response to a stressor, including providing a recombinant Drosophila melanogaster organism including neurons comprising a nucleic acid encoding for a transient receptor potential (TRP) ion-channel polypeptide that is responsive to a particular stressor, where the neurons further include a heterologous nucleic acid encoding a neuropeptide family receptor. The exemplary method further includes exposing the recombinant organism to the stressor and observing the organism's response to the stressor and exposing the organism to the stressor in the presence of a test compound and observing the organism's response to the stressor, where a change in the organism's response to the stressor in the presence of the test compound as compared to the response in the absence of the test compound indicates that the test compound modulates the response to the stressor. The present disclosure also includes recombinant Drosophila organisms as described in the exemplary method.

Yet another exemplary method of identifying a composition capable of reducing a cellular response to a stressor, as provided in the present disclosure, includes exposing a first population of cells to a stressor, wherein the cells include a heterologous nucleic acid encoding a transient receptor potential (TRP) ion-channel polypeptide and a heterologous nucleic acid encoding a neuropeptide receptor, and are in contact with (e.g., exposed to) a fluorescent compound capable of intracellularly fluorescing in the presence of calcium, where the TRP ion-channel polypeptide transports calcium into the cell in response to a stressor. The method further includes delivering to a second population of cells a stressor, where the second population of cells also includes the heterologous nucleic acid encoding the TRP ion-channel polypeptide, and the heterologous nucleic acid encoding the neuropeptide receptor, and is in contact with the fluorescent compound capable of intracellularly fluorescing in the presence of calcium and a test compound. The level fluorescence of the first and second cell populations are determined and compared, and if the level of fluorescence in the first cell population is greater than in the second cell population, the test compound inhibits the uptake of calcium via the transient receptor potential ion-channel polypeptide.

Another exemplary embodiment of the present disclosure includes, among others, a method of identifying a composition capable of reducing a cellular response to a stressor. The exemplary method includes exposing a first population of cells to a stressor, where the cells include a heterologous nucleic acid encoding a transient receptor potential (TRP) ion-channel polypeptide and a heterologous nucleic acid encoding a neuropeptide receptor. The cells are in contact with a medium comprising a neuropeptide capable of selectively binding to the neuropeptide receptor and a fluorescent compound capable of intracellularly fluorescing in the presence of calcium, where the TRP ion-channel polypeptide transports calcium into the cell in response to a stressor. The stressor is also delivered to a second population of cells including the heterologous nucleic acid encoding the TRP ion-channel polypeptide and the heterologous nucleic acid encoding the neuropeptide receptor, where the cells are in contact with a medium including the neuropeptide capable of selectively binding to the neuropeptide receptor, the fluorescent compound capable of intracellularly fluorescing in the presence of calcium, and a test compound. The method further includes determining the difference in the level fluorescence of the first and second cell populations, where if the level of fluorescence in the first cell population is greater than in the second cell population, the test compound inhibits the uptake of calcium via the transient receptor potential ion-channel polypeptide.

An exemplary embodiment of the present disclosure of a recombinant eukaryotic cell, among others, includes a heterologous nucleic acid encoding a transient receptor potential (TRP) ion-channel polypeptide, and a heterologous nucleic acid encoding a neuropeptide Y family receptor.

Other systems, methods, features, and advantages of the present disclosure will be or become apparent to one with skill in the art upon examination of the following drawings and detailed description. It is intended that all such additional systems, methods, features, and advantages be included within this description, and be within the scope of the present disclosure.

### DRAWINGS

Many aspects of this disclosure can be better understood with reference to the following drawings. The components in the drawings are not necessarily to scale. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIGS. 1A to 1G illustrate behavioral paradigms for larval response to aversive food chemicals. FIG. 1A is a digital image showing that post-feeding larvae instinctively migrate away from feeding sites and pupate on the plastic surface of a bottle. FIGS. 1B and 1C are digital images showing that most of w¹¹¹⁸ post-feeding larvae (ca. 96 h AEL, n=25 per plate) moved out of 10% fructose (FIG. 1B) but not water agar paste (FIG. 1C). Images were taken after the larvae pupated. Scale bars, 10 mm. FIG. 1D is a bar graph illustrating quantification of larval aversive responses to different media containing 10% fructose, lactose or sorbitol. FIGS. E and F are digital images illustrating that most of w¹¹¹⁸ post-feeding larvae (ca. 96 h AEL, n=30 per plate) had a sequential display of dispersing, clumping and cooperative burrowing on the solid 10% fructose agar medium within a 30-min period (FIG. 1E). The same larvae showed no clumping or burrowing on water agar medium even after 1.5 hr (FIG. 1F). Images were taken 30 min after the larvae were transferred onto the medium. Scale bars, 10 mm. FIG. 1G is a bar graph showing quantification of larval grouping behavior. The feeding larvae do not show clumping and burrowing activity on solid apple juice agar medium. In all figures, unless otherwise stated, error bars represent standard deviations. At least 3 separate trials were performed for each experiment. Asterisks indicate statistically significant differences from paired controls. P<0.001. ANOVA followed by Student-Newman-Keuls analysis.

FIGS. 2A to 2F illustrate that pain is involved in larval aversion to fruit juice/fructose. The hypomorphic alleles pain¹ and pain³ result from P-element insertions in the 5′ region of the pain gene. pain³ has been shown to confer a stronger defect in thermal sensation than pain¹. FIG. 2A is a bar graph illustrating that larvae with different pain mutant alleles showed reduced aversion to apple juice agar medium (P<0.01). FIG. 2B is a bar graph showing that wild type larvae were aversive to 10% fructose, sucrose and glucose media. The behavioral responses to each sugar were compared between the wild type and each of the pain mutants. Asterisks indicate statistically significant alterations (P<0.01). FIG. 2C is another bar graph illustrating that a transgenic construct containing the genomic sequence of the pain gene (as described in Tracey 2003) restored the food-averse migration in pain³ larvae. P<0.001. FIGS. 2 D and 2E are a digital image and bar graph, respectively, showing that pain³ larvae showed no cooperative burrowing throughout the experiment (>1.5 hr). Scale bar, 10 mm. The rescue construct restored the food-conditioned cooperative burrowing in pain³ larvae. FIG. 2F is a bar graph illustrating that pain³ and wild type larvae showed comparable motilities on water agarose medium (P=0.068).

FIGS. 3A to 3C illustrate conditional disruption of pain-expressing neuronal signaling attenuates larval food aversion. FIG. 3A is a live digital image of a second-instar larva expressing DsRed driven by pain-gal4, which has been shown to recapitulate the endogenous pain expression pattern in the peripheral and central nervous system. Peripheral pain-expressing neurons exist in paired clusters (arrowheads). Scale bar, 200 μm. Gr66a-gal4 has been shown to direct gene expression in larval gustatory neurons of the terminal organs. UAS-shi^{ts1} encodes a semi-dominant-negative form of dynamin that can block neurotransmitter release at a restrictive temperature (>29° C.). FIG. 3B is a bar graph illustrating that at permissive temperature (23° C.), both experimental larvae (pain-gal4 X UAS-shi^{ts1}) and control larvae (e.g., Gr66a-gal4 X UAS-shi^{ts1}) displayed aversive response to apple juice media. At 30° C., the experimental but not control larvae showed attenuated food aversion. P<0.001. FIG. 3C is a bar graph showing that the transgenic larvae crawled at the speed of about 0.6 mm/sec, which is comparable to those of control larvae (pain-gal4 alone or Gr66a-gal4 X UAS-shi^{ts1}).

FIGS. 4A to 4D illustrate that those larvae expressing a mammalian vanilloid receptor display capsaicin-averse behaviors. UAS-VR1E600K encodes a variant of the mammalian capsaicin receptor VR1. FIGS. 4A and 4B are digital images showing that experimental larvae expressing UAS-VR1E600K driven by pain-gal4 migrated away from agar paste containing 25 μM capsaicin (FIG. 4B). Control larvae (e.g., UAS-VR1E600K alone) mostly remained on the capsaicin medium (FIG. 4A). Scale bars, 10 mm. FIG. 4C is a graph showing quantification of avoidance response of transgenic larvae in media containing capsaicin, apple juice or water only. P<0.001. FIG. 4D is a graph illustrating that, in a two-choice assay, pain-gal4 x UAS-VR1E600K feeding larvae (74 h AEL) showed no preference for capsaicin-free media. n>90; P<0.001.

FIGS. 5A to 5H show imaging and SOARS analysis of excitation of thoracic PAIN neurons by fructose with the cameleon Ca²⁺ indicator. Stimulation paradigm: the tissue was initially perfused with HL6-Lac solution for up to 10 min before imaging. During imaging, solutions were changed every 120 seconds, alternating between HL6-Lac and HL6-Fru. FIG. 5A is a composite fluorescent and transmitted light image of pain-expressing neurons from the ventral left cluster (below the Keilin's organ, see also FIG. 7) in the third thoracic segment of the control larva (pain^{gal4}; UAS-YC 2.1). CFP fluorescence is shown in green, and the numbers indicate neurons (anterior, to the left). FIG. 5B is an eigenimage of the above tissue generated from the SOARS analysis of the cameleon YFP/CFP FRET data. This eigenimage facilitates the identification of pixels that display spatially correlated, temporally anti-correlated fluorescence changes selectively responding to fructose stimulation (see Broder, J., et al., 2007). Light (dark) pixels indicate regions where the calcium concentration increased (decreased) in response to fructose. The circled regions containing light pixels correspond to neuronal cell bodies, which display statistically significant periodic responses to fructose (p<10⁻⁵). FIG. 5C is a projection (time-course) of the weighted mask in the data sets showing periodic anti-correlated changes of the CFP and YFP signals. FIG. 5D illustrates the dynamic change of fructose concentration as monitored by flowing 0.0005% fluorescein through the perfusion chamber (blue trace). The black trace indicates the solution switching profile. Note the time lag between on and off switches and the corresponding changes in fluorescein concentration due to connective tubing between the pump and perfusion chamber. Two complete cycles of solution alternation are shown here. FIG. 5E illustrates the periodic change in CFP/YFP ratio in individual neurons responding to the fluctuation in fructose concentration. The strongest response was observed in neuron 4. P<10⁻⁵. In these traces a decrease (increase) in the ratio corresponds with increase (decrease) of calcium concentration. FIGS. 5F-5H represent digital images (FIGS. 5F and 5G) and data analysis (FIG. 5H) of the same set of thoracic pain-expressing neurons from pain mutants (pain^{gal4}/pain³; UAS-YC 2.1) performed using the same procedures described above. No spatially correlated, temporally anti-correlated signals were detected. At least 6 tissues were imaged. Scale bars: 20 μm.

FIGS. 6A to 6E illustrate how ablation of fructose-responsive PAIN neurons in the thoracic segments disrupts larval food aversion. FIG. 6A is a live digital image of the anterior of a third instar larva (74 h AEL) expressing UAS-YC 2.1 driven by pain-gal4. Six clusters of fructose-responsive pain-expressing neurons located on the ventral side of three thoracic segments (T1 to T3) are shown (boxed). Scale bar, 50 μm. FIGS. 6B-6D are magnified digital images of PAIN neurons in the boxed areas from FIG. 6A. There are 6 neurons per cluster in T1 and seven in T2 or T3. Scale bars, 10 μm. FIG. 6E is a bar graph illustrating that, compared to the mock group, ablation of all six neuron clusters or two T2 clusters caused significant reduction in food aversion (n=19, P<0.01). Each experimental and the mock group include at least 18 larvae. Larvae from other experimental groups (e.g., those ablated of one T2 and one T3 cluster) showed no significant behavioral changes (n=18, P>0.08).

FIGS. 7A to 7D illustrate that the ventral PAIN neurons of the three thoracic segments (T1 to T3) project to the thoracic ganglia and denticle belts. The nervous tissues of pain-gal4; UAS-mCD8-GFP larvae (96 h AEL, n=7) were immunostained with anti-GFP antibodies. FIG. 7A is a digital image illustrating that the GFP positive ventral neuron clusters (arrowheads) project to the thoracic ganglia. Scale bar: 50 μm. FIGS. 7B-7D are magnified digital images of neuron clusters in each of the thoracic segments showing their projections to the area near the bristles of the ventral denticle belt (open arrowheads). Scale bar: 20 μm.

FIGS. 8A to 8G illustrate the responses of larval sensory neurons to sugar stimulation. FIGS. 8A-8C illustrate the test of the response of thoracic PAIN neurons to 10% lactose, performed as follows. Briefly, the tissue was initially perfused with HL6-Lac solution for up to 10 min before imaging. During imaging, solutions were changed every 120 seconds, alternating between same HL6-Lac from two separate reservoirs. FIG. 8A is a composite fluorescent and transmitted light image of pain-expressing ventral sensory neurons in third thoracic segment of pain^{gal4}; UAS-YC 2.1 larvae (Anterior to the right). FIG. 8B is an eigenimage of the same tissue generated from the SOARS analysis of the YFP/CFP fluorescence data (Broder, J., et al., 2007 and Fan, X., et al., 2007, each of which are incorporated herein by reference). FIG. 8C illustrates the projection (time-course) of the weighted mask in the data sets showing no significant anti-correlated changes of the CFP and YFP signals. FIGS. 8D-8F illustrate the response of larval chordotonal neurons to fructose stimulation. Solutions were changed in the same fashion, alternating between HL6-Lac and HL6-Fru. FIG. 8D is a composite fluorescent and transmitted light image of pain-expressing chordotonal neurons in first abdominal segment of pain^{gal4}; UAS-YC 2.1 larvae, which are located near the thoracic PAIN neurons imaged in FIG. 5 (Anterior to the left). FIG. 8E is an eigenimage of the same tissue generated from the SOARS analysis. FIG. 8F illustrates the projection (time-course) showing no significant anti-correlated changes of the CFP and YFP signals. FIG. 8G is a graph illustrating the dynamic change of fructose concentration was that monitored by flowing 0.0005% fluorescein through the perfusion chamber (blue trace). The black trace indicates the solution switching profile. Note the time lag between on and off switches and the corresponding changes in fluorescein concentration. Two complete cycles of solution alteration are shown here. Scale bars: 20 μm.

FIGS. 9A to 9E show the localization of NPFR1-positive PAIN neurons. The nervous tissues of pain-gal4 X UAS-DsRed larvae (96 h AEL, n=15) were immunostained with affinity-purified anti-NPFR1 peptide and anti-DsRed antibodies, and imaged using a confocal microscope. FIG. 9A is a digital image showing the anti-NPFR1 antibodies selectively stained a small set of pain-expressing neurons in three thoracic segments (arrowheads), which are absent in larvae expressing an attenuated diphtheria toxin driven by npfr1-gal4. Scale bar, 100 μm. The neurons in the boxed regions are shown below. FIGS. 9B and 9C are magnified images of NPFR1-positive PAIN neurons in the second (T2) and third thoracic (T3) segment, respectively. Scale bars, 30 μm. FIGS. 9D and 9E are digital images of the new anti-NPFR1 peptide antibodies showing an immunofluorescence staining pattern in CNS similar to those of Wu, Q., et al., 2003. A partial stack of confocal images is shown, which include six NPFR1-positive neurons at the dorsomedial surface of the ventral nerve cord (see arrowheads in FIG. 9E, merged) and neuropils of the central brain lobes. NPFR1 expression pattern in the CNS does not appear to overlap with that of DsRed directed by pain-gal4 (see magnified views in FIG. 9E). Scale bars, 50 μm.

FIGS. 10A to 10E illustrate that affinity purified anti-NPFR1 antibody selectively stains cells near Keilin's organs in larval ventral epidermis. UAS-DTI encodes an attenuated diphtheria toxin (see Han, D. D., et al., 2000, which is incorporated herein by reference. FIG. 10A is a digital image showing that NPFR1 immunoreactivity was detected in the ventral epidermis of control larvae (UAS-DTI alone; 96 h AEL; n=12.). Arrowheads indicate the NPFR1 positive cells near the Keilin's organ in the three thoracic segments. The digital image of FIG. 10B illustrates that npfr1-gal4 X UAS-DTI larvae show no specific NPFR1 staining. The dorsal and terminal organs are autofluorescent. n=9. Scale bars, 100 μm. FIGS. 10C-10E are high resolution images of NPFR1-positive cells in the three thoracic segments (T1 T2 and T3), respectively. These cells are located below the cuticle near the keilin's organ (FIGS. 10C to 10E, DIC channels). Scale bars, 20 μm.

FIGS. 11A to 11G illustrate regulation of sugar-stimulated social response by decreased or increased NPFR1 signaling. UAS-npfr1^{dsRNA} encodes an npfr1 double-stranded RNA. FIG. 11A is a digital image showing that, while, young control larvae (74 h AEL) disperse randomly on solid fructose agar coated with a thin layer of 10% fructose yeast paste, at least 70% of younger experimental larvae (pain-gal4/UAS-npfr1dsRNA, 74 h AEL) behaved like older postfeeding larvae, displaying stable aggregation at the edge of the plate (arrows) (Wu et al., 2003). FIG. 11B is a bar graph illustrating quantification of the larval clumping activities from FIG. 1A. P<0.001. UAS-npfr1^{cDNA} and UAS-npf contain an npfr1 and npf coding sequence, respectively. FIG. 11C is a digital image showing that most post-feeding larvae overexpressing NPFR1 in PAIN neurons pupated on apple juice agar paste, while control larvae migrated out of the food medium to pupate on food-free surface. FIG. 11D illustrates quantification of the larval migratory activities. P<0.001. FIG. 11E is a digital image showing that post-feeding control larvae (e.g., UAS-npfr1cDNA/+, 96 h AEL) normally show a sequential display of dispersing, clumping and cooperative burrowing on the solid 10% fructose agar medium within a 30-min period. The arrow indicates a cooperative burrowing site. Images were taken 30 min after the larvae were transferred onto the medium. FIG. 11F illustrates that post-feeding larvae over-expressing NPFR1 in PAIN neurons showed no clumping or burrowing activity on 10% fructose agar medium even after 1.5 hr. FIG. 11G is a bar graph illustrating quantification of the larval clumping activities from. P<0.001. In all figures, unless otherwise stated, error bars represent standard deviations. At least 3 separate trials were performed for each experiment. Asterisks indicate statistically significant differences from paired controls using ANOVA followed by Student-Newman-Keuls analysis.

FIGS. 12A to 12B illustrate that NPFR1 suppresses PAINLESS-mediated thermal nociception in larvae and chemical nociception in adults. FIG. 12A is a bar graph showing that NPFR1 suppresses PAINLESS-mediated thermal nociception in larvae. Most wild type larvae display a stereotypical rolling behavior within 1 sec when touched by a 40° C. probe to the lateral body wall (Tracey et al., 2003). In contrast, the majority of NPFR1 Over-expressers respond after 3 sec. n>100 for each line tested. FIG. 12B is a bar graph showing that NPFR1 suppresses PAIN neuron-mediated chemical nociception in adults. 2-day old control flies mostly avoided 0.4 mM BITC. However, the NPFR1 over-expressing flies showed no preference to either of the two types of food. n>250 for each line tested.

FIGS. 13A to 13D illustrate that NPFR1 suppresses larval avoidance to capsaicin induced by ectopically expressed mammalian TRPV1. UAS-TRPV1 encodes a variant of the mammalian vanilloid receptor TRPV1 (TRPV1E600K). FIG. 13 A is a digital image showing that control larvae expressing UAS-TRPV1 driven by pain-gal4 migrate away from agar paste containing 400 nM capsaicin. FIG. 13B is a digital image illustrating that experimental larvae expressing both NPFR1 and TRPV1 in PAIN cells mostly remained on the capsaicin medium. FIG. 13C is a bar graph representing quantification of avoidance response of transgenic larvae in media containing capsaicin. P<0.001. The bar graph of FIG. 13D shows quantification of capsaicin-induced larval aggregation on the sugar-free capsaicin medium. Larvae expressing TRPV1 alone (paingal4/UAS TRPV1) but not those co-expressing TRPV1 and NPFR1 (pain-gal4/UAS-TRPV1/UAS-npfr1^{cDNA}) showed capsaicin-elicited aggregation. P<0.001.

FIGS. 14A to 14D provide digital imaging and SOARS analysis of excitation of thoracic PAIN neurons by fructose with the cameleon Ca²⁺ indicator. Stimulation paradigm: the tissue was initially perfused with HL6-Lactose solution for up to 10 min before imaging. During imaging, solutions were changed every 120 seconds, alternating between HL6-Lactose and HL6-Fructose. At least 6 tissues per group were imaged. Scale bars: 20 μm. FIG. 14A is a composite fluorescent and transmitted light image of pain-expressing neurons from the ventral left cluster in the third thoracic segment of the NPFR1-overexpressing larva (pain^{gal4}, UASnpfr1^{cDNA}; UAS-YC 2.1). CFP fluorescence is shown in green. FIG. 14B is an eigenimage of the above tissue generated from the SOARS analysis of the cameleon YFP/CFP FRET data. This eigenimage facilitates the identification of pixels that may display spatially correlated, temporally anti-correlated fluorescence changes selectively responding to fructose stimulation (Xu et al., 2008). No spatial-correlated pixels were detected. FIG. 14C illustrates that the time-course of the weighted mask in the data sets shows no periodic anti-correlated changes of the CFP and YFP signals (blue and red traces, respectively). FIG. 14D provides data analysis of the same set of thoracic pain-expressing neurons from control larvae (pain^{gal4}; UAS-YC2.1) that display periodic anti-correlated changes of the CFP and YFP signals under the same conditions as described above. The fructose stimulation paradigm is indicated at the bottom. Three complete cycles of solution alternation are shown here.

FIGS. 15A to 15F illustrate that NPFR1 suppresses Ca²⁺ influx mediated by rat TRPV1 in human cells and presents Ca²⁺ imaging and SOARS analysis of Human Embryonic Kidney (HEK) 293 cells expressing the TRPV1 channel. HEK 293 cells were loaded with the Fluo-4 and Fura-red fluorescent Ca²⁺ indicators, stimulated by 400 nM capsaicin (CAP) and imaged for 300 s. Eigenimages highlight the clusters of pixels showing statistically significant anticorrelated changes in Fluo-4 and Fura-red fluorescence intensities. FIG. 15A is an eigenimage of HEK cells transfected with empty pcDNA3.1 vectors. FIGS. 15 B and 15C are eigenimages of cells transfected with TRPV1 cDNA and stimulated by CAP in the absence or presence of 1 mM NPF. FIGS. 15 D and 15E are eigenimages of cells co-transfected with TRPV1 and NPFR1 cDNAs and stimulated by CAP in the absence or presence of NPF. FIG. 15 F is a graph of SOARS analysis of changes in the ratio between Fluo-4 to Fura-red fluorescence levels during the entire 300-sec recording period. Each trace is generated from at least 3 independent experiments.

FIGS. 16A to 16D illustrate the effects of cyclic nucleotides, PKA and NPFR1, on TRP channel activities. FIG. 16A is a graph illustrating that TRPV1 is sensitized by 8-Br-cAMP and slightly inhibited by 8-Br-cGMP (see line 1, 3 and 5) and that NPFR1 significantly attenuated sensitization of TRPV1 by 8-Br-cAMP (compare line 3 and 4). FIG. 16B is a graph showing that NPFR1 suppression of TRPV1 is enhanced in the presence of 8-BrcGMP (compare line 5 and 6). FIG. 16C is a bar graph illustrating quantification of avoidance response of transgenic larvae in media containing apple juice. Larvae co-expressing PKAc and NPF in PAIN neurons showed attenuated food-averse migration. Most pupated on the medium. P<0.001. FIG. 16D is a bar graph showing quantification of avoidance response of transgenic larvae in sugar-free media. Control larvae that express UAS-PKAc alone pupated mostly outside of the agar medium. Larvae co-expressing PKAc and NPF or NPFR1 in PAIN neurons displayed attenuated food-averse migration. P<0.001.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, organic chemistry, biochemistry, genetics, medicine pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a cell” includes a plurality of cells unless otherwise clearly indicated. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

In describing and claiming the disclosed subject matter, the following terminology will be used in accordance with the definitions set forth below.

The term “isolated cell or population of cells” as used herein refers to an isolated cell or plurality of cells excised from a tissue or grown in vitro by tissue culture techniques. The term “a cell or population of cells” may refer to isolated cells as described above or may also refer to cells in vivo in a tissue of an animal or human.

The term “contacting a cell or population of cells” as used herein refers to delivering a compound, agent, peptide, or the like according to the present disclosure to an isolated or cultured cell or population of cells or administering the compound in a suitable pharmaceutically acceptable carrier or in a growth medium to the target tissue of an animal or human. “Contacting” may include exposing, delivering, and the like. When in reference to an organism, “contacting” the organism with a compound includes administration, delivery, etc. of the compound and/or exposure of the organism to the compound such that the appropriate contact with the compound occurs. Administration may be, but is not limited to, intravenous delivery, intraperitoneal delivery, intramuscularly, subcutaneously, topically, orally or by any other method known in the art.

The term “stressor” as used herein refers to any stimulus which may be applied to a cell or organism that, at some point in the organism's development, produces an adverse response in the cell or organism. Exemplary stressors include, but are not limited to, nociceptive stimuli such as physical pain induced by mechanical stimulation, noxious heat simulation, noxious chemical stimulation (e.g., by capsaicin or isothiocyanate, and the like, and even sugar in some instances), or neuropathic pain (e.g., enhanced sensitivity to benign stimuli and/or abnormal sensitivity to mild noxious stimuli, and spontaneous pain). In some cases, especially with higher organisms, stressors may include, stimuli such as mental or physical stress (such as, but not limited to, stress and other neurological effects of emotional trauma and/or physical hardship (e.g., starvation, and the like) or a combination of both emotional and physical stress. When used in reference to a multicellular organism herein, “adverse response” generally indicates avoidance of the stressor or other physical manifestation by the organism of a negative or abnormal response to a stimulus (including, but not limited to, movement, migration, cooperative behaviors, and the like as indicated by the circumstances). However, when used in reference to a cell or population of cells (isolated or in-vivo) an “adverse response” may include an indicator of nociceptive stimuli visible on the cellular level, such as intracellular calcium uptake via a TRP ion-channel polypeptide.

As used herein, the term “inhibit,” “decrease,” and/or “reduce” generally refers to the act of reducing, either directly or indirectly, a function, activity, or behavior relative to the natural, expected, or average or relative to current conditions. For instance, if an agent inhibits or reduces a response to a stressor, it reduces or eliminates the occurrence of the response to a particular stressor (e.g., a stimulus) that would be expected to or has been demonstrated to occur in the absence of the agent. For instance, if a certain action or compound is said to reduce an aversion response in a Drosophila melanogaster larvae to a particular stimulus, this indicates the occurrence or the extent of the aversive response is less than what would be expected if the action or compound had not been administered. With respect to decreasing or inhibiting expression of a peptide, this may indicate downregulation of the peptide.

As used herein, the term “modulate,” “modify” and/or “modulator” generally refers to the act of promoting/activating or interfering with/inhibiting a specific function or behavior. In some instances a modulator may increase or decrease a certain activity or function relative to its natural state or relative to the average level of activity that would generally be expected. For instance, a modulator of a response to a stressor might increase, decrease or otherwise change a response to a stressor from what would otherwise be the expected response. A modulator may act by causing the overexpression or underexpression of a peptide (e.g., by acting to upregulate or downregulate expression of the peptide), or it may directly interact with the subject peptide to increase and/or decrease activity.

The term “dye” or “fluorescent compounds” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). Suitable fluorophores(chromes) for the use in the present disclosure may be selected from, but not intended to be limited to, Fluo-4 and Fura-red fluorescent dyes, and the like.

The term “polymerase chain reaction” or “PCR” as used herein refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

The term “polymerase” as used herein refers to an enzyme that catalyzes the sequential addition of monomeric units to a polymeric chain, or links two or more monomeric units to initiate a polymeric chain. In advantageous embodiments of this invention, the “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

The term “primer” as used herein refers to an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand.

The term “protein” as used herein refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

The term “target” as used herein refers to a peptide, cell, tissue, tumor, etc, for which it is desired to detect. The target peptide may be on a cell surface, the cell being isolated from an animal host, a cultured cell or a cell or population of cells in a tissue of an animal.

The term “peptide” or “polypeptide” as used herein refers to proteins and fragments thereof. Peptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term “variant” refers to a peptide or polynucleotide that differs from a reference peptide or polynucleotide, but retains essential properties. A typical variant of a peptide differs in amino acid sequence from another, reference peptide. Generally, differences are limited so that the sequences of the reference peptide and the variant are closely similar overall and, in many regions, identical. A variant and reference peptide may differ in amino acid sequence by one or more modifications (e.g., substitutions, additions, and/or deletions). A variant of a peptide includes conservatively modified variants (e.g., conservative variant of about 75, about 80, about 85, about 90, about 95, about 98, about 99% of the original sequence). A substituted or inserted amino acid residue may or may not be one encoded by the genetic code. A variant of a peptide may be naturally occurring, such as an allelic variant, or it may be a variant that is not known to occur naturally.

The present disclosure includes peptides which are derivable from the naturally occurring sequence of the peptide. A peptide is said to be “derivable from a naturally occurring amino acid sequence” if it can be obtained by fragmenting a naturally occurring sequence, or if it can be synthesized based upon knowledge of the sequence of the naturally occurring amino acid sequence or of the genetic material (DNA or RNA) that encodes this sequence. Included within the scope of the present disclosure are those molecules which are said to be “derivatives” of a peptide. Such a “derivative” or “variant” shares substantial similarity with the peptide or a similarly sized fragment of the peptide and is capable of functioning with the same biological activity as the peptide.

A derivative of a peptide is said to share “substantial similarity” with the peptide if the amino acid sequences of the derivative is at least 80%, at least 90%, at least 95%, or the same as that of either the peptide or a fragment of the peptide having the same number of amino acid residues as the derivative.

The derivatives of the present disclosure include fragments which, in addition to containing a sequence that is substantially similar to that of a naturally occurring peptide may contain one or more additional amino acids at their amino and/or their carboxy termini. Similarly, the invention includes peptide fragments which, although containing a sequence that is substantially similar to that of a naturally occurring peptide, may lack one or more additional amino acids at their amino and/or their carboxy termini that are naturally found on the peptide.

The disclosure also encompasses the obvious or trivial variants of the above-described fragments which have inconsequential amino acid substitutions (and thus have amino acid sequences which differ from that of the natural sequence) provided that such variants have an activity which is substantially identical to that of the above-described derivatives. Examples of obvious or trivial substitutions include the substitution of one basic residue for another (i.e. Arg for Lys), the substitution of one hydrophobic residue for another (i.e. Leu for Ile), or the substitution of one aromatic residue for another (i.e. Phe for Tyr), etc. Modifications and changes can be made in the structure of the peptides of this disclosure and still obtain a molecule having similar characteristics as the peptide (e.g., a conservative amino acid substitution). For example, certain amino acids can be substituted for other amino acids in a sequence without appreciable loss of activity. Because it is the interactive capacity and nature of a peptide that defines that peptide's biological functional activity, certain amino acid sequence substitutions can be made in a peptide sequence and nevertheless obtain a peptide with like properties.

In making such changes, the hydropathic index of amino acids can be considered. The importance of the hydropathic amino acid index in conferring interactive biologic function on a peptide is generally understood in the art. It is known that certain amino acids can be substituted for other amino acids having a similar hydropathic index or score and still result in a peptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant peptide, which in turn defines the interaction of the peptide with other molecules, such as enzymes, substrates, receptors, antibodies, antigens, and the like. It is known in the art that an amino acid can be substituted by another amino acid having a similar hydropathic index and still obtain a functionally equivalent peptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent peptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent peptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, H is), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu).

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompass a polynucleotide as defined above.

In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically, or metabolically modified forms of polynucleotides, as well as the chemical forms of DNA and RNA characteristic of viruses and cells, including simple and complex cells, inter alia.

By way of example, a polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminus positions of the reference nucleotide sequence or anywhere between those terminus positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

As used herein, DNA may be obtained by any method. For example, the DNA includes complementary DNA (cDNA) prepared from mRNA, DNA prepared from genomic DNA, DNA prepared by chemical synthesis, DNA obtained by PCR amplification with RNA or DNA as a template, and DNA constructed by appropriately combining these methods.

As used herein, an “isolated nucleic acid” is a nucleic acid, the structure of which is not identical to that of any naturally occurring nucleic acid or to that of any fragment of a naturally occurring genomic nucleic acid spanning more than three genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in random, uncharacterized mixtures of different DNA molecules, transfected cells, or cell clones, e.g., as these occur in a DNA library such as a cDNA or genomic DNA library.

The DNA encoding the proteins and peptides disclosed herein can be prepared by the usual methods: cloning cDNA from mRNA encoding the protein, isolating genomic DNA and splicing it, chemical synthesis, and so on. cDNA can be cloned from mRNA encoding the protein by, for example, the method described as follows:

First, the mRNA encoding the protein is prepared from the above-mentioned tissues or cells expressing and producing the protein. mRNA can be prepared by isolating total RNA by a known method such as guanidine-thiocyanate method (Chirgwin et al., Biochemistry, 18:5294, 1979), hot phenol method, or AGPC method, and subjecting it to affinity chromatography using oligo-dT cellulose or poly-U Sepharose.

Then, with the mRNA obtained as a template, cDNA is synthesized, for example, by a well-known method using reverse transcriptase, such as the method of Okayama et al (Mol. Cell. Biol. 2:161 (1982); Mol. Cell. Biol. 3:280 (1983)) or the method of Hoffman et al. (Gene 25:263 (1983)), and converted into double-stranded cDNA. A cDNA library is prepared by transforming E. coli with plasmid vectors, phage vectors, or cosmid vectors having this cDNA or by transfecting E. coli after in vitro packaging.

The term “substantially pure” as used herein in reference to a given polypeptide indicates that the polypeptide is substantially free from other biological macromolecules. For example, the substantially pure polypeptide is at least 75%, 80, 85, 95, or 99% pure by dry weight. Purity can be measured by any appropriate standard method known in the art, for example, by column chromatography, polyacrylamide gel electrophoresis, or HPLC analysis. As used herein, the term “purified” and like terms relate to the isolation of a molecule or compound in a form that is substantially free (at least 60% free, preferably 75% free, and most preferably 90% free) from other components normally associated with the molecule or compound in a native environment.

The term “host” or “organism” as used herein includes humans, mammals (e.g., cats, dogs, horses, etc.), insects, living cells, and other living organisms. A living organism can be as simple as, for example, a single eukaryotic cell or as complex as a mammal. Typical hosts to which embodiments of the present disclosure relate will be insects (e.g., Drosophila melanogaster) mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For some applications, hosts may also include plants. For diagnostic or research applications, a wide variety of mammals will be suitable subjects, including rodents (e.g., mice; rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. Hosts that are “predisposed to” condition(s) can be defined as hosts that do not exhibit overt symptoms of one or more of these conditions but that are genetically, physiologically, or otherwise at risk of developing one or more of these conditions.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. Typically the introduced exogenous sequence is a recombinant sequence. While an exogenous nucleic acid sometimes originates from a different organism or species, it may also originate from the same species. For instance, an exogenous sequence may be extra copy or recombinant form of a nucleic acid introduced into a cell or organism in addition to or as a replacement for the naturally occurring nucleic acid. Or, the exogenous sequence may be a sequence derived from the same source, but placed in a location in which it is not normally found. For instance, an exogenous nucleic acid may include a nucleic acid taken from one type of cell in an organism and expressed in a different type of cell in the organism, in which it is generally not found.

The term “heterologous” typically indicates derived from a separate genetic source, a separate organism, or a separate species. Thus, a heterologous nucleotide is nucleotide from a first genetic source expressed by a second genetic source. The second genetic source may be a vector. In some instances “heterologous” may indicate something derived from the same source, but that has been placed in a location in that source where it is not typically found. For instance, a “heterologous” nucleic acid may include a nucleic acid taken from one location or type of cell in an organism and expressed in a different type of cell in the organism, in which it is generally not found.

The term “operably linked” refers to the arrangement of various nucleotide sequences relative to each other such that the elements are functionally connected to and are able to interact with each other. Such elements may include, without limitation, one or more promoters, enhancers, polyadenylation sequences, and transgenes. The nucleotide sequence elements, when properly oriented, or operably linked, act together to modulate the activity of one another, and ultimately may affect the level of expression of the transgene. For example, control sequences or promoters operably linked to a coding sequence are capable of effecting the expression of the coding sequence, and an organelle localization sequence operably linked to protein will direct the linked protein to be localized at the specific organelle. The position of each element relative to other elements may be expressed in terms of the 5′ terminus and the 3′ terminus of each element, and the distance between any particular elements may be referenced by the number of intervening nucleotides, or base pairs, between the elements.

A “vector” is a genetic unit (or replicon) to which or into which other DNA segments can be incorporated to effect replication, and optionally, expression of the attached segment. Examples include, but are not limited to, plasmids, cosmids, viruses, chromosomes and minichromosomes. Exemplary expression vectors include, but are not limited to, baculovirus vectors, modified vaccinia Ankara (MVA) vectors, plasmid DNA vectors, recombinant poxvirus vectors, bacterial vectors, recombinant baculovirus expression systems (BEVS), recombinant rhabdovirus vectors, recombinant alphavirus vectors, recombinant adenovirus expression systems, recombinant DNA expression vectors, and combinations thereof.

A “coding sequence” is a nucleotide sequence that is transcribed into mRNA and translated into a protein, in vivo or in vitro.

“Regulatory sequences” are nucleotide sequences, which control transcription and/or translation of the coding sequences that they flank.

The term “transform” or “transformation” refers to permanent or transient genetic change induced in a cell following incorporation of exogenous DNA. As used herein, a transformed cell, host cell, or population of cells generally refers to a cell into which (or into an ancestor of which) has been introduced, by means of recombinant DNA techniques, a heterologous polynucleotide.

The term “overexperss” and/or “over-expression” indicates that a particular peptide is expressed (e.g., by a cell) in a greater amount than would normally be expected. For instance transformation of a cell with heterologous DNA that results in the cell having additional copies of the DNA or transformation with heterologous DNA under the control of an inducible or consitutative promoter are methods used to induce over-expression of a peptide encoded by the heterologous DNA.

As used herein, the terms “treatment”, “treating”, and “treat” are defined as acting upon a disease, disorder, or condition with an agent to reduce or ameliorate the pharmacologic and/or physiologic effects of the disease, disorder, or condition and/or its symptoms. “Treatment,” as used herein, covers any treatment of a disease in a host (e.g., a mammal, typically a human or non-human animal of veterinary interest), and includes: (a) reducing the risk of occurrence of the disease in a subject determined to be predisposed to the disease but not yet diagnosed as infected with the disease (b) impeding the development of the disease, and (c) relieving the disease, i.e., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of a disease or pathogen inhibiting agent that provides for enhanced or desirable effects in the subject (e.g., reduction of pathogen load, reduction of disease symptoms, etc.).

As used herein, the terms “prophylactically treat” or “prophylactically treating” refers completely or partially preventing a disease or symptom thereof and/or may be therapeutic in terms of a partial or complete cure for a disease and/or adverse effect attributable to the disease.

As used herein the term “test compound” may include peptides, peptidomimetic, a chemical, and a nucleic acid sequences. In some embodiments the “test compound” may be a compound, such as a chemical or peptide that is suspected of having a modulating effect on a biological response to a particular stressor. In other embodiments, a library of multiple “test compounds” may be examined for a modulatory effect on a particular stressor.

### Discussion

Embodiments of the present disclosure include methods of identifying compounds capable of modulating and, in particular, inhibiting, a response to a stressor.

Such methods include methods utilizing a Drosophila melanogaster model, as well as in vitro methods utilizing recombinant cell lines. The present disclosure also includes recombinant cell lines for use in the methods of the present disclosure and for further understanding of the chemical and neural pathways associated with biological responses to various stimuli.

All animals must cope with stress. In mammals, opioids and adrenalins are effective agents for treating certain types of stress. As demonstrated in the present disclosure, NPY family peptides, which are widely conserved among various species, play a role in stress and pain responses. NPF, the sole member of the NPY family in Drosophila melanogaster, is also involved in the fly's ability to cope with certain stressors. Also evolutionarily conserved, the transient receptor potential (TRP) family of cation channels represents sensors of diverse stressful stimuli. For instance, mammalian TRPV1 responds to noxious heat, protons and capsaicin, and plays an important role in nociception. The Drosophila TRPA channel protein PAINLESS (PAIN) (e.g., SEQ ID NO. 1, accession No. NM_{—}138135) (Nucleic acid sequence SEQ ID NO. 2) is implicated for fly aversive responses to thermal, mechanical and chemical stressors and also plays a developmental role in the behavioral changes in larval response to food.

The present disclosure describes how NPF and other NPY family peptides promote resistance to diverse stressors through a pathway involving neuropeptide receptors for NPY family peptides. NPF suppresses PAIN-mediated sugar aversion throughout early larval development in Drosophila, and loss of NPF signaling in feeding larvae triggers precocious sugar aversion behaviors. Further, the examples below demonstrate that NPFR1, a G-protein coupled receptor for NPF, (e.g., SEQ ID NO: 3, accession No. NM_{—}079521) (DNA SEQ ID NO: 4) suppressed avoidance behaviors of fly larvae mediated by different subtypes of TRP channels. The present disclosure also illustrates that NPFR1 exerts this inhibitory effect by reducing Ca²⁺ influx mediated by fly TRPA and mammalian TRP channels in cultured human cells. These findings and the broad distribution of different TRP channel proteins in central and peripheral neurons indicate that NPF/NPFR1 signaling system is representative of a broad and ancient stress-coping mechanism that reduces response to stressful stimulation by attenuating different TRP family channels. The methods and cells of the present disclosure utilize the NPY/NPYR (e.g., NPF/NPFR1) system to identify compounds capable of inhibiting a response to a stressor in a host.

In an embodiment, a method of the present disclosure includes a method of identifying a composition capable of inhibiting a response to a stressor by using a Drosophila model system. Prior to metamorphosis, post-feeding Drosophila larvae migrate away from the feeding habitat and burrow into food-free soil for pupation. This developmentally regulated habitat switching prevents immobile pupae from drowning or killing by microorganisms inside food proper. The Drosophila NPY family member, NPF (e.g., SEQ ID NO: 5, accession No. AAF55339) (DNA SEQ ID NO: 6), temporally controls larval habitat switching. NPF activity in the brain of feeding larvae is high, but precipitously down-regulated in older larvae exiting the feeding phase. Prolonged brain expression of NPF in post-feeding larvae suppressed food aversion, while premature reduction of NPF signaling in feeding larvae elicited precocious food-averse behaviors normally associated with older larvae, thereby indicating that NPF inhibits the food aversion behavior in feeding larvae.

As discussed in more detail in the examples below, fructose-averse behaviors in Drosophila are mediated by a subset of sensory neurons on the ventral side of larval thoracic body segments that express the TRPA channel protein, PAIN. Importantly, NPFR1 appears to be selectively expressed in the fructose-responsive but not other PAIN sensory neurons. Since the NPF/NPFR1 pathway is such a potent inhibitor of fly nociceptors, fly larva normally have it only when it is needed for growth and survival (e.g., to allow larva to stay in a fructose-rich medium during the feeding phase). The studies described in the present application demonstrate that NPF/NPFR1 acts selectively on PAINLESS signaling only in certain places (e.g., selective neurons), making it possible for other PAIN neurons (e.g., those that are NPFR1-negative) to be responsive to other aversive stimuli (e.g., noxious heat).

Thus, NPF, which is highly expressed in the central nervous system (CNS) of feeding larvae suppresses larval sugar aversion by directly silencing the NPFR1-positive PAIN neurons. Adult flies also demonstrate aversion to isothiocyanate (a chemical compound found in horseradish and wasabi). As shown in the examples, NPFR1 over-expression also desensitizes adult flies to noxious chemical stimulus with isothiocyanate. Further, as shown in the examples, the NPFR1 protein can be introduced into other types of PAIN sensory neurons in both fly and mammalian cells by introduction of a heterologous nucleic acid encoding for the NPFR1 protein (SEQ ID NO. 3) where it interacts with NPF to suppress aversive behaviors mediated by noxious stimuli applied to the transformed PAIN neurons.

Since the present disclosure demonstrates that the Drosophila NPF/NPFR1 pathway has also been shown to suppress mammalian TRP channels (e.g., rat TPRV1), to function in mammalian cells, and to exhibit parallel activity to the NPY/NPYR pathway in higher organisms, a Drosophila model represents a simple and inexpensive way to screen for potential inhibitors of stressors in higher organisms, including but not limited to mammals (e.g., humans). Thus, the present disclosure presents methods of using a Drosophila model system to identify potential inhibitors for various stressors.

In embodiments of the present disclosure, a method of the disclosure for identifying a composition capable of inhibiting a response to a stressor includes exposing (e.g., introducing, contacting, and the like) a Drosophila melanogaster organism to a medium containing a compound that elicits an avoidance response in a wild-type Drosophila organism, where the Drosophila organism exhibits an avoidance response to the medium, and contacting (e.g., exposing, introducing, and the like) the larva with a test compound, wherein a reduction in the avoidance response to the medium in the presence of the test compound as compared to in the absence of the test compound indicates that the test compound modulates response to a stressor in a higher organism. Additional description regarding procedures for comparing Drosophila behavior is provided in the examples below. The compound contained in the medium can include, but is not limited to a chemical that produces an avoidance behavior in Drosophila, although the chemical might only cause the avoidance behavior during certain phases of Drosophila development. For instance, adult flies show avoidance to isothiocyanate, a compound found in horseradish and wasabi. Thus, if an adult fly is used in the method, the medium may contain a chemical such as isothiocyanate or other chemical compound that produces aversion in adult flies. In the larval stage, post-feeding Drosophila avoid sugar, so if larval Drosophila are used, the medium may contain sugar, such as fructose, or a other fructose-containing medium, such as apple juice medium. The test compound can be, but is not limited to, a chemical, peptide, nucleic acid, or other compound suspected of suppressing the stressor. In embodiments, a library of test compounds can be screened to determine if any compounds suppress the sugar avoidance response in Drosophila, indicating the compound(s) may modulate a response to a stressor in a higher organism (e.g., animal or human). In embodiments the test compound interacts with Drosophila NPF1 to inhibit TPRA activity. In embodiments the test compound mimics and/or modulates NPF activity.

In exemplary embodiments, a method of the disclosure for identifying a composition capable of inhibiting a response to a stressor includes exposing a larval form of a Drosophila melanogaster to a sugar medium, where the Drosophila larva exhibits an avoidance response to the medium, and contacting the larva with a test compound, wherein a reduction in the avoidance response to the sugar medium in the presence of the test compound as compared to in the absence of the test compound indicates that the test compound modulates response to a stressor in a higher organism. Additional description regarding procedures for comparing Drosophila behavior is provided in the examples below. The sugar medium can include, but is not limited to, a fructose-containing medium, such as apple juice medium.

In other exemplary embodiments of the methods of the disclosure, an adult Drosophila fly is exposed to a medium containing a chemical that induces an avoidance response in Drosophila. The chemical may include, but is not limited to, a noxious compound such as isothiocyanate. The fly is also contacted with a test compound, where a reduction in the avoidance response to the medium containing the noxious compound in the presence of the test compound, as compared to in the absence of the test compound indicates that the test compound modulates response to a stressor in a higher organism. The test compound may be contained in the medium, or it may be administered to the Drosophila by other methods known to those of skill in the art, such as by injection, topical administration, subcutaneous, oral, or in the form of an expression vector if the test compound is a peptide or nucleic acid that may be produced in vivo. Additional details regarding these methods are included in the examples below and are known to those of skill in the art.

In embodiments of the methods of the disclosure, the Drosophila organism (e.g., adult fly or larval form) may be modified to over-express fly NPFR1 (e.g., SEQ ID NO: 3), thereby enhancing the effect of a suppression activity by a test compound that acts by binding NPFR1. As discussed above, exemplary methods to achieve over-expression of a NPFR1 polypeptide include, but are not limited to, transforming the organism with additional copies of NPFR1, such as by use of an expression vector, or transforming the organism or cell with a recombinant nucleotide encoding for NPFR1 that is operably linked to a constitutive promoter. Additional methods for over-expressing a peptide in an organism are known to those of skill in the art, and exemplary embodiments are provided in the examples below and/or are incorporated by reference.

In other embodiments, recombinant Drosophila organisms (e.g., larvae or adult flies) are produced by modifying the organism to express heterologous NPFR1 (e.g., SEQ ID NO. 3) in PAIN neurons that do not express NPFR1 in wild-type flies. For example, as described in greater detail below, recombinant Drosophila larvae (e.g., pain-gal4 X UAS-npfr1^{cDNA} larvae) were produced that ectopically expressed NPFR1 in the entire set of PAIN neurons including those that are responsive to noxious heat, whereas wild-type flies typically express NPFR1 only in PAIN neurons responsive to sugar. These pain-gal4 X UAS-npfr1^{cDNA} larvae displayed attenuated aversive response to noxious heat not observed in wild-type flies. Such recombinant organisms are useful for screening for modulators of TRP ion-channel activated by various stressors.

Thus, in an exemplary embodiment, a method of identifying a composition capable of inhibiting a response to a stressor includes providing a recombinant Drosophila melanogaster organism with PAIN neurons including a nucleic acid encoding for a transient receptor potential (TRP) ion-channel polypeptide that is responsive to a particular stressor (e.g., noxious heat, noxious chemicals, mechanical stimulus, and the like) and also including a heterologous nucleic acid encoding a neuropeptide family receptor (e.g., NPFR1 from Drosophila). The method further includes exposing the recombinant Drosophila to the stressor and observing the organism's response to the stressor, exposing the organism to the stressor in the presence of a test compound and observing the organism's response to the stressor. A change in the organism's response to the stressor in the presence of the test compound as compared to the response in the absence of the test compound indicates that the test compound modulates the response to the stressor. The heterologous nucleic acid encoding a neuropeptide family receptor can be any nucleic acid encoding any neuropeptide family receptor capable of suppressing TRP ion-channel polypeptides. Exemplary neuropeptide family receptors include, but are not limited to NPFR1 from Drosophila (e.g., SEQ. ID NO. 3), and other NPY family receptors, such as those from higher organisms, including mammals. In exemplary embodiments, the TRP ion-channel polypeptide is a Drosophila TRPA peptide sensitive to noxious heat stimulus (e.g., SEQ ID NO. 1), and the stressor is a heat probe. Recombinant flies can be produced by methods known to those of skill in the art and as generally described in the examples below. Thus, the present disclosure also includes embodiments of a recombinant Drosophila melanogaster organism including a heterologous nucleic acid encoding for a neuropeptide receptor present in neural cells, that don't typically express the neuropeptide receptor. These neural cells in also include a TRP ion-channel polypeptide is responsive to a particular stressor. In embodiments the recombinant Drosophila organisms according to the present disclosure may include, but are not limited to, a Drosophila organism including a heterologous nucleic acid encoding for a neuropeptide receptor polypeptide found in Drosophila, but not expressed in those particular cells (e.g., Drosophila NPFR1, e.g., SEQ ID NO. 3), or a neuropeptide receptor from a different organism, such as, but not limited to, a mammal (e.g., an NPY receptor).

Additionally, the examples demonstrate that heterologous expression of rat TRPV1 (e.g., SEQ ID NO. 7) in post-feeding larvae in PAIN neurons was sufficient to trigger larval aversion to capsaicin media, and post-feeding larvae co-expressing NPFR1 and TRPV1 in PAIN neurons displayed no capsaicin-averse behaviors. Also, younger feeding larvae expressing rat TRPV1 have been shown to be insensitive to capsaicin (Xu et al., 2008 Nature Neuroscience 11: 676-682, which is incorporated herein by reference). These results illustrate that TRP signaling in other organisms, particularly mammals, can also be blocked by NPFR1.

Thus, another embodiment of a method of the present disclosure for identifying a composition capable of modulating a response to a stressor, includes providing a recombinant form of a Drosophila melanogaster (e.g., larva or adult flies) comprising a heterologous transient receptor potential (TRP) ion-channel polypeptide from a different organism, where the TRP ion-channel polypeptide is responsive to a particular stressor; exposing the larva to the stressor, where the Drosophila organism exhibits an avoidance response to the stressor; and contacting the larva with a test compound, where a reduction in the avoidance response to the stressor in the presence of the test compound as compared to in the absence of the test compound indicates that the test compound modulates response to a stressor in a higher organism. Recombinant Drosophila organisms can be produced as described in the examples below, and as taught in the art. In an exemplary embodiment of the disclosure, the heterologous TRP ion-channel polypeptide is a rat TRPV1. In an embodiment where the heterologous TRP ion-channel polypeptide is rat TRPV1, the stressor is capsaicin.

Thus, the present disclosure also includes embodiments of a recombinant Drosophila melanogaster organism including a heterologous transient receptor potential (TRP) ion-channel polypeptide from a different organism, where the TRP ion-channel polypeptide is responsive to a particular stressor. For example, embodiments of recombinant Drosophila organisms according to the present disclosure may include, but are not limited to, a Drosophila organism including a heterologous nucleic acid encoding for a TRP ion-channel polypeptide from a mammal (e.g., TRPV1 from rat, e.g., SEQ ID NO. 7).

Additionally, a recombinant cell or cell line expressing a heterologous transient receptor potential (TRP) ion-channel polypeptide and a heterologous neuropeptide receptor would be useful for in vitro screening for compounds that inhibit a response to a stressor. In particular, using recombinant mammalian cells, and even human cells, further validates the potential suppressive action of a test compound in a higher organism. As described in more detail in the examples, Drosophila NPFR1 cDNA and rat TRPV1 were co-expressed in mammalian cells (HEK293 cells). In these cells, NPFR1 potently suppressed TRPV1 signaling activity. Control cells (TRPV1 alone, TRPV1/NPFR1 without NPF, or TRPV1 with only NPF) responded to capsaicin within 30 sec, and TRPV1 channels appeared to remain active for at least five minutes (the entire test period). However, experimental cells (NPFR1/TRPV1 with NPF) showed greatly attenuated response to capsaicin.

Thus, embodiments of the present disclosure include a recombinant eukaryotic cell including a heterologous nucleic acid encoding a transient receptor potential (TRP) ion-channel polypeptide and a heterologous nucleic acid encoding a neuropeptide Y family receptor. In embodiments the cell is a mammalian cell (e.g., rat, primate, human, etc.). In a particular embodiment the cell is a human cell and/or cell line (e.g., HEK293 cells). Suitable cell lines useful for providing the recombinant cells of the present disclosure are known to those of skill in the art. Methods of producing the recombinant cells of the present disclosure include, but are not limited to, stably transferring the heterologous nucleic acids to the cell line via methods known in the art (e.g., use of expression vectors, and the like). Exemplary methods of producing the recombinant cells of the present disclosure are provided in the examples below.

In embodiments of the recombinant cell according to the present disclosure, the TRP ion-channel polypeptide is selected from TRP ion-channel polypeptides including, but not limited to, Drosophila TRPA (e.g., SEQ. ID. NO: 1) and rat TRPV1 (e.g., SEQ ID NO: 7). In embodiments, the neuropeptide Y family receptor includes, but is not limited to, Drosophila NPRF1 (e.g., SEQ. ID NO: 3). In embodiments the neuropeptide includes, but is not limited to, a neuropeptide Y family member, such as, but not limited to NPF from Drosophila melanogaster (e.g., SEQ ID NO: 5). In another embodiment the neuropeptide is a Y-family member peptide from a mammal (e.g., humans).

In another embodiment, cellular imaging can be used to examine how the NPF/NPFR1 (or NPY/NPY receptor) signaling pathway affects the intracellular uptake of Ca²⁺ via TRP family channels. Example 2 demonstrates that Ca²⁺ uptake is reduced in cells and larvae expressing NPFR1, demonstrating the NPFR1 mediated suppression of TRP activity. Thus, observation of cellular Ca²⁺ uptake provides another method for identifying potential suppressors of TRP activity and thus, compounds capable of inhibiting/reducing a response to a stressor.

In an embodiment, a method of identifying a composition capable of reducing a cellular response to a stressor includes providing a population of cells that include a heterologous nucleic acid encoding a transient receptor potential (TRP) ion-channel polypeptide and a heterologous nucleic acid encoding a neuropeptide receptor, where the TRP ion-channel polypeptide transports calcium into the cell in response to a stressor (e.g., noxious heat, mechanical stimulation, noxious chemicals (e.g., capsacisn, isothiocyanate, fructose, etc., as appropriate). The cells can be exposed to a fluorescent compound capable of intracellularly fluorescing in the presence of calcium. When the cells are exposed to the stressor, the TRP ion-channel polypeptide transports calcium into the cell in response to the stressor. Fluorescence imaging technology can be used to detect fluorescence, which indicates activity of the TRP ion-channel polypeptide. A second population is also provided and is exposed to the same stressor as well as a test compound, and observed for differences. The second population of cells also includes the heterologous nucleic acid encoding the TRP ion-channel polypeptide, and the heterologous nucleic acid encoding the neuropeptide receptor, and is in contact with the fluorescent compound capable of intracellularly fluorescing in the presence of calcium and the test compound. The level fluorescence of the first and second cell populations are determined and compared, and if the level of fluorescence in the first cell population is greater than in the second cell population, the test compound inhibits the uptake of calcium via the transient receptor potential ion-channel polypeptide. Fluorescent compounds and detection methods and technology useful in the methods of the present disclosure include those described in the examples below, as well as those known to those of skill in the art and described herein. In an embodiment the fluorescent compounds include, but are not limited to Fluo-4 and Fura-red fluorescent dyes, and the like.

This observation of cellular TRP activity by measuring intracellular Ca²⁺ levels also provides methods for screening for additional compounds that modulate NPFR1/NPF mediated suppression of TRP activity. As described in Example 2, a cGMP analog, 8-BR-cGMP, was found to synergistically enhance the NPFR1/NPF suppression of TRP ion-channel activity. Thus, this represents another method for screening for compounds that directly and/or indirectly inhibit response to a stressor via modulation of the NPFR1/NPF/TRP pathway. Such compounds may modulate the NPF/NPFR1 pathway by directly interacting with NPF and/or NPFR1 and/or TRP. Such modulators may increase or decrease the suppression of intracellular Ca²⁺ uptake by TRP channels. Preferably, such compounds would act to enhance the suppression mediated by the NPF/NPFR1 pathway, thereby representing a potential compound for modulating stressors in a host needing treatment for a stressor (e.g., physical pain, as well as mental and physical stress and symptoms thereof).

Another embodiment of the present disclosure includes a method of identifying a composition capable of reducing a cellular response to a stressor, including exposing a first population of cells and a second population of cells to a stressor, and testing the effect of a test compound on the response to the stressor in the second population of cells. The first set of cells include a heterologous nucleic acid encoding a TRP ion-channel polypeptide and a heterologous nucleic acid encoding a neuropeptide receptor and are in contact with a medium including a neuropeptide capable of selectively binding to the neuropeptide receptor and a fluorescent compound capable of intracellularly fluorescing in the presence of calcium. As discussed above and demonstrated in the examples, the TRP ion-channel polypeptide transports calcium into the cell in response to a stressor. The second population of cells also include the heterologous nucleic acid encoding the TRP ion-channel polypeptide and the heterologous nucleic acid encoding the neuropeptide receptor, and the cells are in contact with a medium including the neuropeptide capable of selectively binding to the neuropeptide receptor, and the fluorescent compound capable of intracellularly fluorescing in the presence of calcium. The medium in contact with the second population of cells also includes a test compound. Then, the difference in the level fluorescence of the first and second cell populations is determined, and if the level of fluorescence in the first cell population is greater than in the second cell population, the test compound inhibits the uptake of calcium via the transient receptor potential ion-channel polypeptide. This method is useful for screening for compounds that modulate the inhibitory effect of NPF, such as, but not limited to, 8-BR-cGMP, a cGMP analog, and other compounds that directly or indirectly modulate the NPFR1/NPF/TRP signaling pathway.

In the above embodiments that employ a population of cells including heterologous nucleic acids encoding TRP peptides and heterologous nucleic acids encoding neuropeptide receptors, the TRP ion-channel polypeptide may be, but is not limited to, Drosophila TRPA and rat TRP1, as described above. In embodiments, the neuropeptide Y family receptor includes, but is not limited to, Drosophila NPRF1 and other neuropeptide Y family members. In embodiments where the cells are in contact with a neuropeptide capable of selectively binding to the neuropeptide receptor, the neuropeptide may include, but is not limited to, NPF from Drosophila and other NPY neuropeptides.

Now having described the embodiments of the present disclosure, in general, Examples 1 and 2, below, describe some additional embodiments of the present disclosure. While embodiments of the present disclosure are described in connection with Examples 1-2 and the corresponding text and figures, there is no intent to limit embodiments of the present disclosure to these descriptions. On the contrary, the intent is to cover all alternatives, modifications, and equivalents included within the spirit and scope of embodiments of the present disclosure.

### EXAMPLES

### Example 1

### Drosophila TRPA Channel PAINLESS Modulates Sugar-Stimulated Neuronal Excitation, Avoidance and Social Interaction

The contents of this Example are also described in Xu J, Sornborger A T, Lee J K, Shen P (2008) Drosophila TRPA channel modulates sugar-stimulated neural excitation, avoidance and social response. Nat Neurosci 11:676-682, which is incorporated herein by reference in its entirety.

D. melanogaster post-feeding larvae display food-averse migration towards food-free habitats prior to metamorphosis. This developmental switching from food attraction to aversion is regulated by a neuropeptide Y (NPY)-related brain signaling peptide. The present example utilizes the fly larva model to delineate the neurobiological basis of age-restricted response to environmental stimuli. This data provides evidence for a fructose-responsive chemosensory pathway that modulates food-averse migratory and social behaviors. This demonstrates that fructose potently elicits larval food-averse behaviors, and PAINLESS (PAIN), a TRPA channel responsive to noxious stimuli, is involved in the fructose response. A subset of pain-expressing sensory neurons have been identified that display PAIN-dependent excitation by fructose. Although evolutionarily conserved avoidance mechanisms are widely appreciated for their roles in stress coping and survival, their biological significance in animal physiology and development remains underexplored. The present findings demonstrate how an avoidance mechanism is recruited to facilitate animal development.

### Introduction

Sensory systems, which define an animal's perception of its own world, are of primary importance to behavioral development and adaptation. It has been observed in both vertebrate and invertebrate species that an organism may restrict or modify its behavioral response to a particular sensory stimulus in an age-dependent manner. However, regulatory mechanisms underlying developmentally programmed modifications of natural behaviors remain to be better understood.

The genetically tractable D. melanogaster larva offers a useful model to investigate how an animal modulates its chemosensory properties and behaviors in coordination with development. Third-instar fly larvae display two opposing food responses: younger larvae live mostly inside aqueous food media such as overripe fruits and apple juice-agar paste; in contrast, older post-feeding larvae avoid food media and display migration (also known as wandering) towards food-free sites such as soils or plastic surface for pupation. New post-feeding larvae also display a social response to aversive food stimuli; these larvae instinctively aggregate on harder apple juice-agar media and dig cooperatively through the food proper. These food-conditioned migratory and social behaviors are likely beneficial to the survival of pupae by minimizing their exposure to harmful microorganisms and drowning in the feeding habitat such as rotten fruits.

Neuropeptide Y (NPY), an abundant signaling peptide in the brain of mammals, has been implicated in diverse physiological processes and behaviors including food and alcohol response and the suppression of anxiety and pain. NPY family signaling peptides have been found in organisms ranging from humans to worms. The genome of D. melanogaster encodes a single member of the NPY family, neuropeptide F (NPF). The brain expression of NPF is high in younger third instars that live mostly inside food media, but is rapidly downregulated in new post-feeding larvae. Prolonged NPF expression in older larvae is sufficient to block the developmental onset of food-averse migratory and social behaviors and extend the feeding phase. Conversely, attenuated NPF signaling in younger feeding larvae triggers precocious display of food-averse behaviors normally associated with wandering larvae. These findings suggest the Drosophila NPY-like system may be a developmental regulator of larval food aversion.

The conserved transient receptor potential (TRP) family ion channel proteins are polymodal receptors capable of responding to diverse stressful stimuli including noxious chemicals, light, heat and touch. The well-characterized mammalian vanilloid receptor TRPV1 has been shown to respond to noxious heat, protons and capsaicin, a spicy substance in hot chili peppers. The D. melanogaster genome contains at least 13 TRP family members including the pain gene, which mediates sensation of noxious heat and mechanical touch in fly larvae. Thus, TRP channel proteins may play a role in larval sensation of aversive food chemicals.

This example demonstrates that the developmental onset of food-averse migratory and social behaviors in D. melanogaster larvae is regulated by a chemosensory neuronal pathway responsive to fructose. A TRPA channel protein, PAIN, is essential for larval chemosensory response to fruit juice or fructose. Furthermore, a subset of larval sensory neurons that display PAIN-dependent excitation by fructose have been identified, and targeted ablation of these neurons abolished larval food aversion. These findings illustrate that the larval behavioral switch from food attraction to aversion may require modulation of a PAIN-dependent peripheral sensory module by a temporal control module involving brain NPF signaling.

### Methods

Flies, media and larval growth. Conditions for rearing adult flies and egg collection are described in the following references, which are incorporated herein by reference in their entirety. (Shen and Cai, 2001; Wen et al., 2005; Roberts 1986). The larvae were raised at 25° C. with exposure to natural lighting. Synchronized eggs were collected within a 2 h interval, and late second instars were transferred to a fresh apple-juice plate with yeast paste (<80 larvae per plate). The pain-rescue, pain¹, pain³, pain^{gal4}, Gr66a-gal4 and UAS-shi^{ts1}, UAS-VR1E600K, UAS-YC2.1 lines are described in the following references, which are incorporated herein by reference in their entirety. (Tracey et al., 2003; Kitamoto, 2002; Marella, S., et al., 2006; Liu et al., 2003; Wen et al., 2005).

Behavioral Assays. The food aversion assay was performed in plastic petri dishes (60 mm in diameter). Each apple juice-agar plate contains a mix (ca. 7 ml) of 1 g of Drosophila agar powder (USB, Swampscott, Mass.) and 6 ml apple-juice solution (0.1 g carbohydrates per ml, equivalent to 20% of frozen concentrate). Other soft agar media were made by mixing 1 g agar powder with 6.5 ml of distilled water or solution containing 10% fructose, 10% lactose, 10% sorbitol or 50 μM capsaicin, respectively. The amount of agar powder may require adjustment depending on agar quality. Twenty-five new post-feeding larvae (96 h AEL) were transferred onto a plate. The larvae were allowed to move freely on the medium, and those that crawled onto the plastic surface became less mobile and eventually formed pupae there. The percent of pupae on agar media was scored after 24 hours. All experiments were performed at room temperature except for the temperature shift assay. In these experiments, larvae and food media were pre-incubated at 30° C. for 60 min before the assay. The larvae were subsequently transferred onto the medium and kept at 30° C. All assays were performed in the dark. At least three separate trials were performed per assay.

The locomotion test was performed in a plate (87 mm in diameter) containing 1.3 g agarose powder mixed with 8.7 ml of distilled water. Larvae were rinsed repeatedly to remove visible food particles. Eight larvae were allowed to crawl on the medium, and their locomotion activities were recorded by a SONY DCR-HC36 video camera. The video clip was converted to 1 frame s⁻¹ by iMovie HD, and imported into the Image J software. The track lengths were calculated using the MTrack2 plug-in and converted to speed. At least 20 individual larvae were tested for each data point. All data were analyzed using one-way ANOVA, followed by the Student-Newman-Keuls analysis.

The two-choice medium preference assay was performed on a 2.5% agar plate. The apple juice or water agar paste is the same as described above. 10 μl of 5 mM capsaicin stock solution (in 100% ethanol) or 10 μl ethanol was added to each ml of the agar media. 30 larvae were washed extensively to remove any food particles. They were then placed between the two piles of capsaicin and capsaicin-free media (1 cm in diameter), which are located 4 mm apart. The number of larvae in each medium was recorded after 20 min. A preference index was defined as the fraction of larvae choosing the capsaicin-free medium, minus the fraction of larvae choosing the capsaicin medium. A preference index close to +1 indicates that the larvae are attracted to the tested medium, whereas −1 indicates strong rejection. The larvae outside of both media (typically 5%) were excluded from the calculation.

Laser ablation. Selected pain-expressing sensory neurons were ablated using a 337 nm nitrogen laser unit (Spectra-Physics, Model VSL337NDS, Irvine, Calif.). Power calibration was performed by focusing laser beam onto a mirror. The graduated neutral density filter was adjusted until the reflective layer of the mirror can be penetrated by a single shot. The filter was then moved 4 stops toward the clear end. Early third instar larvae (pain-gal4 X UAS-YC 2.1, ca. 78 h AEL) were rinsed briefly and placed onto a coverslip, and then anesthetized by CO₂ for 10 min. The immobilized larvae were transferred onto a microscope slide with larval ventral side facing straight up. 100 μl of ether was added to a piece of absorbent paper sandwiched between the slide and cover slip to keep the larvae immobile during laser ablation, which typically takes about 10 min.

To ablate the neurons, the laser beam was focused to the nucleus. 4 bursts of 10 shots were fired at a repetition rate of 10 shots/s. Ablated neurons showed reduced GFP intensity, and became invisible after 24 h. After ablation, each individual larva was allowed to recover on a 35 mm soft apple juice agar plate with 30 μl yeast paste on the surface. The pupation site was recorded after 48 h. Larvae from the mock group were handled and anesthetized in the same manner as experimental larvae except without laser treatment. The mortality rates of the control and experimental groups were similar (32.4% vs. 36.7%). The survived larvae developed into adults normally. Data was analyzed using an unpaired Student t-test.

Immunohistochemistry. Larval epidermis was filleted from the dorsal side. The CNS and epidermal tissues were fixed according to a previously published protocol with some modifications³. The fixation time was 35 min. Tissues were washed in PBS with 0.4% TritonX-100, and permeabilized with Proteinase K digestion and post-fixation. Tissues blocked in 10% BSA were incubated overnight at 4° C. with mouse anti-DsRed (1:250, BD Biosciences, San Jose, Calif.) and affinity-purified rabbit anti-NPFR1 antibodies (1:100). The NPFR1 peptide antibodies were raised and purified using two peptide antigens (CMTGHHEGGLRSAIT and SSNSVRYLDDRHPLC). Alexa 488-conjugated anti-rabbit IgG and Alexa 568-conjugated anti-mouse IgG secondary antibodies were diluted to 1:2,000.

Imaging. The live images of second instar larvae (ca. 48 h AEL) expressing DsRed driven by pain-gal4 were obtained using a Leica epifluorescence microscope. The immunofluorescence images were taken by a Zeiss LSM 510 Meta confocal microscope and processed by Zeiss AIM Image Examiner. Calcium imaging of sensory neurons was performed using third instar larvae (ca. 96 h AEL) transgenic for the genetically encoded yellow cameleon Ca²⁺ indicator (UAS-YC2.1) under the pain promoter. Larvae were dissected in a modified HL6 solution containing lactose (HL6-Lac, 23.7 mM NaCl, 15 mM MgCl₂, 24.8 mM KCl, 0.5 mM CaCl₂, 10 mM NaHCO₃, 5 mM HEPES, 240 mM lactose)³⁸. An incision was made along the dorsal midline, and the gut and the fat bodies were removed. The tissue was placed ventral side up in HL6 solution on a silicon-coated coverslip (Sylgard 184, DOW Corning Corp., Midland, Mich.) in a Dvorak-Stotler perfusion chamber (Lucas Highland, Chantilly, Va.). A minute amount of cyanoacrylate glue (Nexaband S/C, Abott Laboratories, North Chicago, Ill.) was applied to the edge of the cuticle using a glass micropipette to immobilize the tissue. The imaging was performed on a Zeiss LSM 510 Meta confocal microscope (Carl Zeiss Microimaging, Thornwood, N.Y.). YC2.1 was excited at 458 nm with an argon laser. Emission fluorescence was filtered by a BP 475-525 filter (cyan) and an LP 530 filter (yellow). 256×256 pixel images for ratiometric analysis were collected at 1 frame s⁻¹. The tissue was perfused at a rate of 8.3 ul s⁻¹ with HL6-Lac, periodically alternating with a modified HL6 solution containing fructose (HL6-Fru, with 240 mM fructose substituting 240 mM lactose in HL6-Lac), switching at 120-second intervals for a total of 800 seconds. The images were subsequently analyzed with the SOARS method (Statistical Optimization for the Analysis of Ratiometric Signals, Version 1.1) as described in the following references, which are hereby incorporated by reference herein in their entireties (Fan, X., et al., 2007; Broder, J., et al., 2007), using Matlab (MathWorks, Natick, Mass.; also see http://www.engr.uga.edu/research/groups/atslab/Software.html).

In brief, SOARS is a multivariate statistical optimization procedure performed on both CFP and YFP channels of the FRET imaging data. The analysis results in a set of eigenimages and associated time courses that represent the part of the imaged signal displaying statistically significant spatial correlation and temporal anti-correlation (e.g., the aspects of the signal that are demonstrable due to FRET). These eigenimages represent the spatial distribution of anti-correlated FRET changes in response to sugar stimulation. Because the stimulus was periodic, the time courses were tested for the presence of stimulus-locked activity. To quantify the significance of the periodic part of the FRET response, p-values for a periodic (sinusoidal) response at the stimulus frequency were calculated using multitaper harmonic analysis (a common method for the detection of sinusoids in noisy data) as described in the following references, which are hereby incorporated herein by reference in their entirety. (Thomson 1982; Mitra et al., 1999, Sornborger et al., 2003; Mitra et al., 2008).

### Results

Behavioral Paradigms for Larval Food Aversion

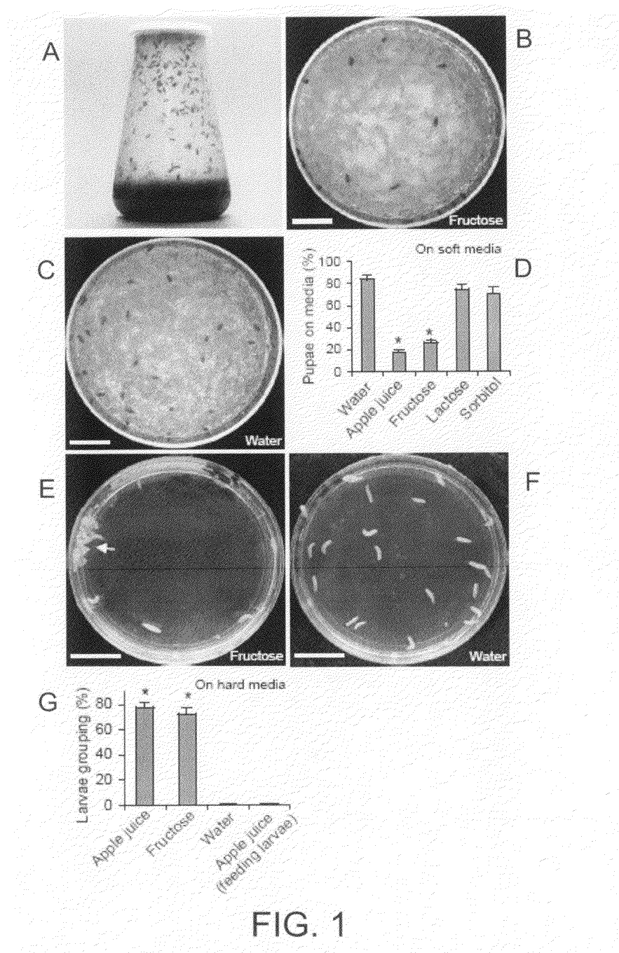

Under controlled laboratory conditions, new post-feeding larvae (ca. 96 hr after egg laying, 96 hr AEL) display migration towards food-free plastic surfaces prior to metamorphosis (FIG. 1A). Two behavioral paradigms were employed to quantitatively assess larval behavioral response to food media. In the first assay, larval migratory response was measured by placing 25 new post-feeding larvae on the center surface of apple juice-containing soft agar media. A majority of normal w¹¹¹⁸ larvae (˜85%) moved away from the apple juice medium within two hours, and they became less mobile and subsequently pupated on a dry plastic surface. In one set of experiments, apple juice was replaced with 10% fructose in the agar paste. Again, almost 80% of larvae displayed the wandering behavior and selected pupation sites outside of the fructose medium (FIG. 1B). In contrast, on water agar paste, larvae browsed randomly and remained in the medium. Consequently, about 80% larvae were found to form pupae that were embedded in the surface layer (FIG. 1C). Moreover, 10% lactose or sorbitol was not effective in triggering larval wandering (FIG. 1D), suggesting that larvae are responsive to external gustatory stimulation by fructose, the most abundant sugar in many overripe fruits, rather than to osmotic pressure.

On a harder apple juice-agar surface, new post-feeding larvae exhibited rapid aggregation, which provides the synergy that enables larvae to dig efficiently through a hard food medium. Since fruit fly larvae exiting rotten fruits display a strong preference to quickly burrow into the pupation habitat (moist soil), this instinctive cooperative behavior may conceivably facilitate larval penetration of fruit juice-stained compact soil near the fallen fruits. The present data also demonstrate that post-feeding larvae (96 hr AEL) displayed aggregation and cooperative burrowing on solid 10% fructose but not water agar media (FIG. 1E-G). These results suggest that fructose can also trigger the grouping behavior of larvae on harder surfaces.

Pain Mediation of Larval Aversion to Fruit Juice/Fructose

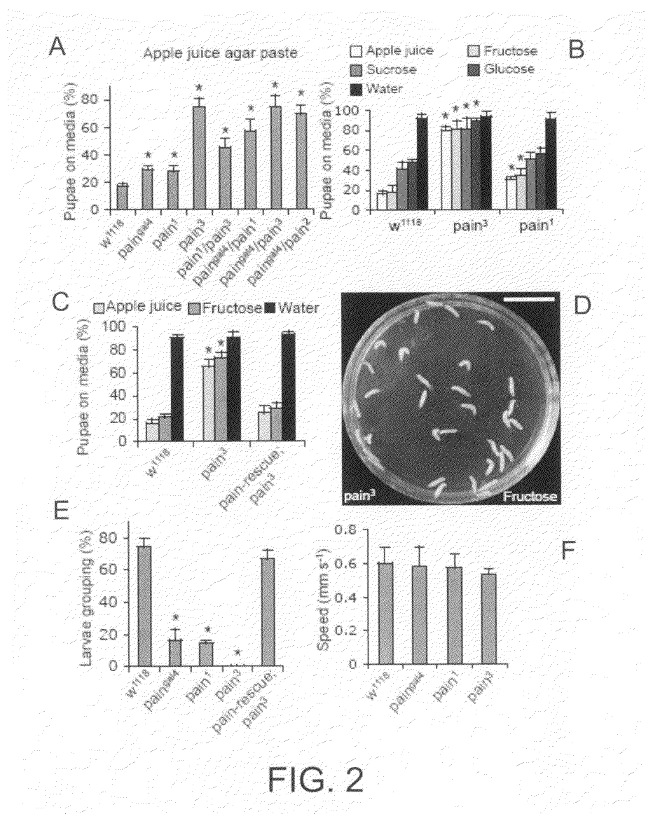

The pain gene is involved in the sensation of noxious heat and mechanical touch in third-instar larvae of D. melanogaster. This example investigated whether pain may play a role in larval food-averse migration. The effects of four mutant alleles of pain on larval wandering behavior were tested. For example, it was found that pain³ larvae were largely insensitive to the apple juice medium, with about 75% remaining on the medium (FIG. 2A). Also, pain¹ larvae showed smaller but significant deficits in migration. The trans-heterozygous larvae (e.g., pain¹/pain³) exhibited an intermediate food-averse response. These results are consistent with the findings that pain³ larvae displayed stronger defects in noxious heat response relative to pain¹ (see Tracey, 2003), which is hereby incorporated by reference herein). Other trans-heterozygous larvae also exhibited significant deficits in migration. The pain³ larvae were also largely insensitive to 10% fructose and other sugar media (FIG. 2B), and a transgenic construct containing an 8.5-kb genomic sequence of pain rescued the mutant phenotype (FIG. 2C).

The behavioral response of pain³ larvae to solid 10% fructose agar media was further tested. These larvae browsed randomly on both 10% fructose and water agar media, and no aggregation and burrowing activities were observed (FIGS. 2D and E). Importantly, pain³ and wild type larvae showed comparable locomotor activities on the water agar medium (FIG. 2F). For example, pain³ larvae crawled at a speed of about 0.5 mm/sec, which could allow a larva to move across an assay plate in 3 min, suggesting that the mutant phenotypes of pain³ larvae are unlikely due to a locomotor defect. Taken together, these results indicate that the TRP channel protein PAIN is important for larval chemosensory response to aversive fructose in the feeding habitat.

Conditional Disruption of PAIN Neuronal Signaling by shibire^{ts1}

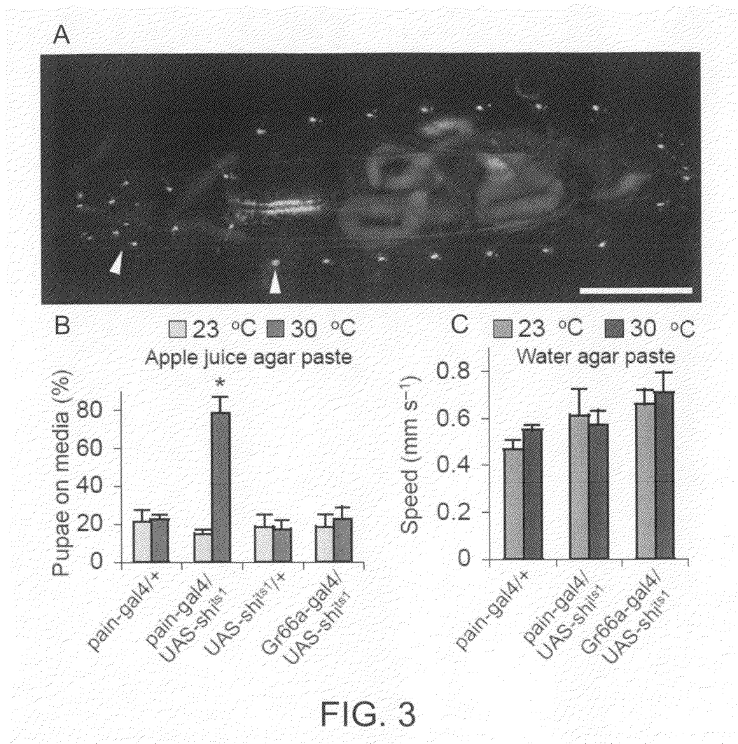

The pain^{gal4} allele contains a GAL4 coding sequence inserted immediately downstream of the pain promoter (see Tracey (2003) above). This pain-gal4 driver has been shown to direct reporter expression in the PAIN cells of the central and peripheral nervous system (CNS and PNS (FIG. 3A). To conditionally disrupt the activity of PAIN neurons, pain-gal4 was used to express a temperature-sensitive allele of shibire (shi^{ts1}), which encodes a semi-dominant negative form of dynamin capable of blocking neurotransmission at a restrictive temperature (>29° C.)²¹. At 23° C., both experimental (pain-gal4 X UAS-shi^{ts1}) and control larvae (e.g., UAS-shi^{ts1} alone) displayed similar wandering activities on apple juice media. However, a majority of experimental larvae remained in the food medium at 30° C., while most of control larvae migrated away (FIG. 3b). The locomotor activity of pain-gal4 X UAS-shi^{ts1} larvae was similar to those of controls (FIG. 3c). These findings suggest that the signaling activity of PAIN neurons is directly involved in maintaining the food-averse response in wandering larvae.

Larval Aversion to Capsaicin Triggered by Mammalian TRPV1

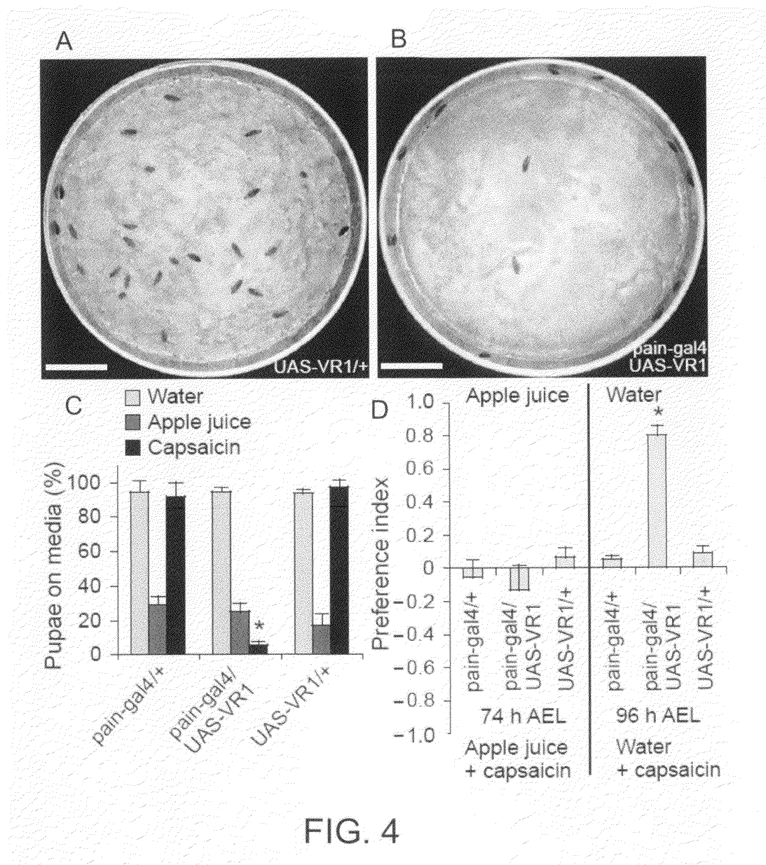

To provide further evidence that larval wandering behavior represents an aversive response to fructose, pain-gal4 was used to express a mammalian TRP channel protein VR1E600K, a variant of vanilloid receptor TRPV1 responsive to a spicy substance capsaicin from chili peppers (see the following references, which are incorporated herein by reference in their entireties: Caterina, M. J., et al., 1997; Marella, S., et al., 2006; Tobin, D. M., et al., 2002). The majority of control larvae (e.g., VR1E600K alone) browsed and eventually pupated on the agar paste containing 25 μM capsaicin, showing no aversive response to the medium (FIG. 4A). However, virtually all of the post-feeding larvae expressing VR1E600K migrated away from the capsaicin-agar medium (FIGS. 4B and C). Moreover, in a two-choice assay, post-feeding VR1E600K-expressing larvae displayed preference for capsaicin-free media while younger feeding VR1E600K-expressing larvae showed no such preference (FIG. 4D). These data strongly support the notion that fructose is an aversive chemical to post-feeding larvae, and peripheral PAIN sensory neurons are responsible for the migratory response to aversive chemical cues.

Abolishing Fructose Response of Thoracic Sensory Neurons by Pain Mutations

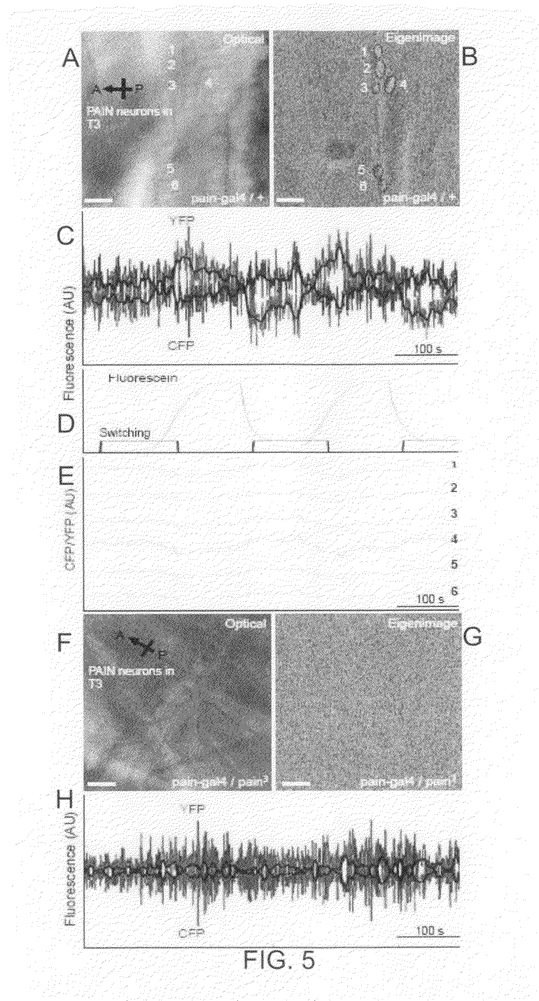

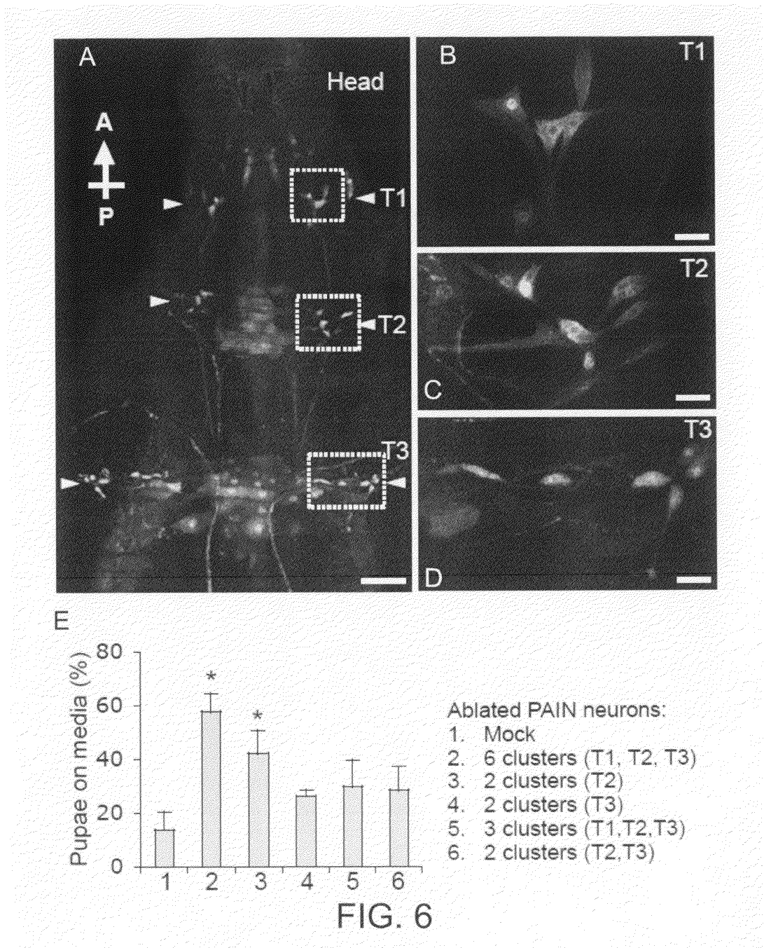

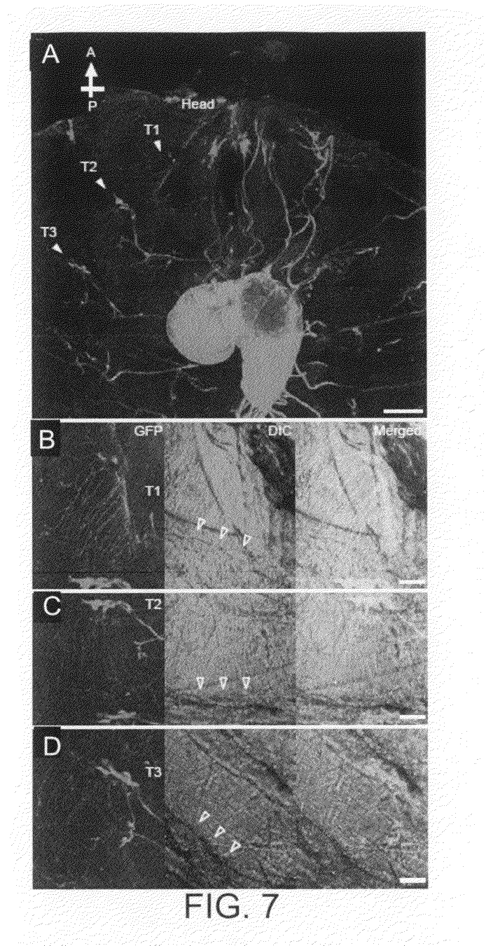

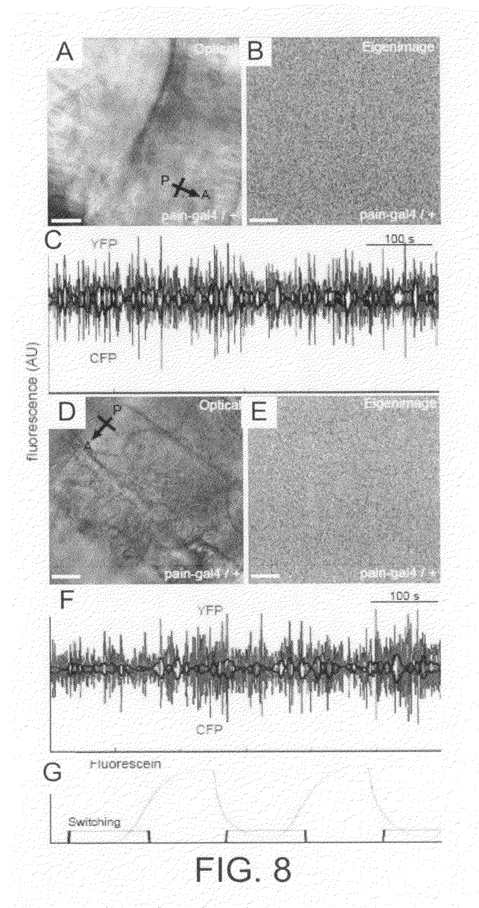

Pain neurons are present widely in the larval PNS. Since larvae crawling on a flat surface of apple-juice agar display the aversive response, it was believed that pain may mediate sugar stimulation in those sensory neurons located in the ventral side of the larval body. Of particular interest are six clusters of pain-expressing ventral neurons in the three thoracic segments (see FIG. 6A and FIG. 7). The somata of these neurons are located near the Keilin's organs (the presumed primordial legs of larvae), and their processes are projected directly into the thoracic ganglia and the ventral denticle belts. Neuronal excitation was imaged with a Ca²⁺-sensitive fluorescent protein, yellow cameleon 2.1 (YC2.1) as described in Liu, L. et al., 2003, which is hereby incorporated by reference herein in its entirety. Initial imaging studies provided evidence for fructose-stimulated intracellular Ca²⁺ increases in three pairs of clustered neurons from each of the thoracic segments in normal third-instar larvae (pain^{gal4}/UAS-YC2.1; 96 h AEL). Analysis was then focused on the PAIN neurons in the second and third thoracic segments. The imaging results of PAIN neurons from the third thoracic segment are shown as an example in FIGS. 5A-E. It was found that these neurons in pain^{gal4}/UAS-YC2.1 larvae were stimulated by lactose-to-fructose but not lactose-to-lactose switch. Moreover, the nearby pain-expressing chordotonal neurons were not responsive to the fructose treatment nor the thoracic Pain neurons from younger feeding larvae (76 h AEL; see Table 1 and FIG. 8). On the other hand, the same thoracic PAIN neurons in pain mutants (pain^{gal4}/pain³; UAS-YC2.1) showed no significant Ca²⁺ increases upon fructose stimulation (FIGS. 5F-H and Table 1). These results indicate that pain is involved in the excitation of these thoracic sensory neurons by fructose.

TABLE 1
Summary of the calcium imaging data.

| | | | | Anti- | |
|---|---|---|---|---|---|
| | | Stimulation | Neuron cluster | correlated | No. of |
| Line | Age | padigram | examined | signal | tissues |
| pain^{gal4}/+; | 96 h | Lactose-fructose, | Ventral sensory | Yes | 8 |
| UAS-YC 2.1/+ | AEL | alternate, 3 repeats | neurons in T2 and T3 | p < 1 × 10⁻⁵ | |
| | | | segments | | |
| pain^{gal4}/+; | 74 h | Lactose-fructose, | Ventral sensory | No | 9 |
| UAS-YC 2.1/+ | AEL | alternate, 3 repeats | neurons in T2 and T3 | | |
| | | | segments | | |
| pain^{gal4}/pain³; | 96 h | Lactose-fructose, | Ventral sensory | No | 8 |
| UAS-YC 2.1/+ | AEL | alternate, 3 repeats | neurons in T2 and T3 | | |
| | | | segments | | |
| pain^{gal4}/+; | 96 h | Lactose-lactose, | Ventral sensory | No | 8 |
| UAS-YC 2.1/+ | AEL | alternate, 3 repeats | neurons in T2 and T3 | | |
| | | | segments | | |
| pain^{gal4}/+; | 96 h | Lactose-fructose, | Chordotonal neurons | No | 7 |
| UAS-YC 2.1/+ | AEL | alternate, 3 repeats | in A1 segment | | |

Disruption of Food Aversion by Ablating Thoracic PAIN Neurons

Selective ablation of the fructose-responsive PAIN neurons was performed to determine if this could abolish food-averse behaviors. Simultaneous ablation of all six clusters of ventral PAIN neurons in the three thoracic segments (T1 to T3) effectively disrupted larval aversion to apple-juice media (FIG. 6E). Moreover, partial ablation of two of the six clusters in the second thoracic segment was sufficient to severely disrupt larval food aversion. However, asymmetric ablation of one of the two clusters in two or all three thoracic segments had at most marginal effect on larval food aversion. These results indicate that the fructose-responsive PAIN neurons in the thoracic segments, especially those in the T2 segment, are involved in larval food aversion.

NPFR1 Expression in Peripheral PAIN Neurons

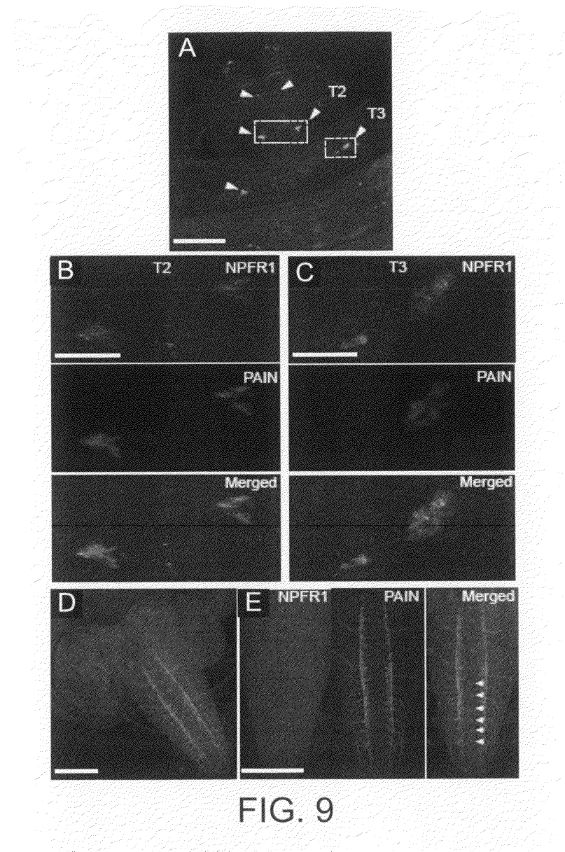

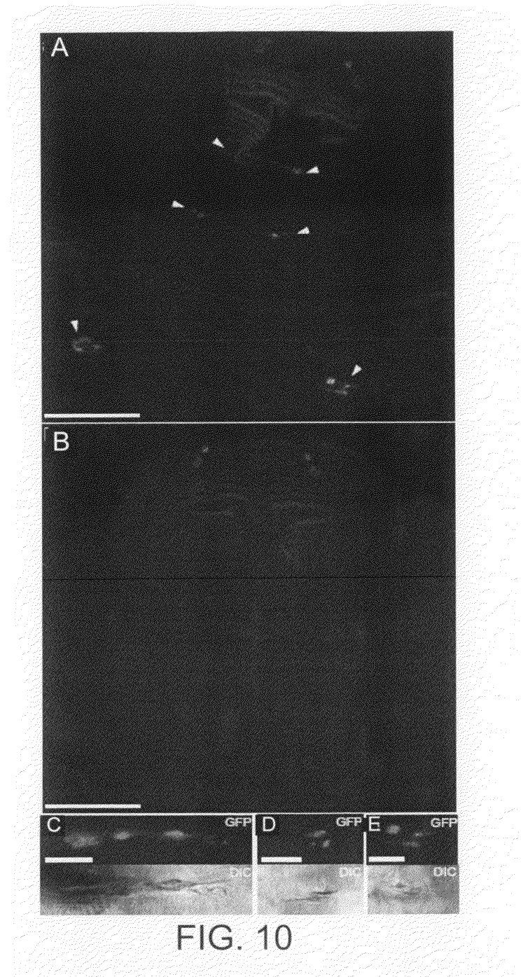

To determine whether PAIN neurons could potentially respond to NPF directly, the location of NPFR1 expression in the nervous system was examined. The intact CNS and epidermis tissues of larvae (96 h AEL) expressing DsRed driven by pain-gal4 were immunostained with both anti-DsRed and anti-NPFR1 peptide antibodies. It was found that NPFR1 immunoreactivity co-localizes with DsRed in three pairs of clustered pain-expressing neurons near the Keilin's organs in the ventral epidermis of the thoracic segments (see FIG. 9 and FIG. 10). These results raise the possibility that fructose-responsive PAIN neurons may be directly regulated by NPF. NPFR1 immunoreactivity was also detected in the somata and neuropils of the brain lobes, subesophageal ganglia and ventral nerve cord. However, it does not appear to overlap with pain-expressing cells in the CNS (FIG. 9E). Importantly, mammalian Y1 and Y2, which are most closely related to NPFR1, have also been found in the nociceptors of the dorsal root ganglia and spinal neurons. Thus, the present findings suggest another potential parallel activity between NPF and NPY in the suppression of stressful sensation.

### Discussion

The present example provides both neuroanatomical and functional evidence that the developmental onset of migratory and social behaviors in the wandering larvae of D. melanogaster is stimulated by aversive stimulants (e.g., fructose) from the feeding habitat, and the conserved nociceptive gene pain is involved in fructose stimulation of thoracic sensory neurons and fructose-conditioned aversive behaviors. NPF, the sole fly homolog of human NPY, is highly expressed in the brain of feeding larvae but downregulated in new wandering larvae; thus, it was considered to potently suppresses larval aversion to apple juice media. The present data indicates that the developmental switch from food attraction to aversion of wandering larvae is regulated by a conserved neural signaling network involving a TRPA-like peripheral sensory module and an NPY-like central module for temporal control. These results also provide a rare example of how an avoidance mechanism has been recruited during evolution to facilitate animal development.

The present results have revealed that a subset of thoracic PAIN sensory neurons directly projects to thoracic ganglia and the areas near the bristles of the ventral denticle belts. Loss-of-function pain mutations completely abolished the fructose-responsive intracellular calcium increase in these neurons. Therefore, the ventral projections of these fructose-responsive neurons are likely present to allow them to be in direct contact with the medium surface that wandering larvae crawl on, making them well suited to mediate larval food-averse response. These findings also suggest that fly larvae use two separate chemosensory pathways for sensing sugars. The appetitive response to sugar is likely to be mediated by maxillary or terminal organs, whereas the aversive response to sugar may be mediated by sensory organs on the ventral thoracic surface. Although thoracic PAIN neurons expressing DsRed in each of the clusters showed comparable red fluorescence levels, some of the neurons in the cluster showed significantly less immunofluorescent signals when stained with anti-DsRed antibodies. Therefore, it is possible that some PNS neurons may be less accessible to antibodies, and the actual number of NPFR1-positive neurons could also be higher.

The following example adds to these findings by helping to elucidate how fructose triggers excitation of PAIN neurons in the thoracic neurons. The PAIN ion channel could act as a promiscuous receptor for diverse chemicals such as fructose and isothiocyanate. Alternatively, it may mediate the signaling activity of a yet uncharacterized member of the seven-transmembrane gustatory receptor family. Interestingly, a significant number of gustatory receptor-expressing neurons (e.g., Gr66a neurons) in the fly have been found to express pain. The functional significance of PAIN in gustatory neurons for food tasting remains to be determined.

NPF suppresses food-averse behaviors in feeding larvae, and its neural signaling activity is regulated developmentally. The present example demonstrates that in feeding larvae, pain-expressing ventral neurons in the thoracic segments do not respond to fructose. It was also observed that NPFR1 overexpression directed by pain-gal4 suppressed the onset of larval food-averse behaviors as well as other sensory functions of abdominal PAIN neurons that normally do not express NPFR1 (unpublished data). The present finding that fructose-responsive thoracic PAIN neurons are positive for NPFR1 immunoreactivity suggests that NPF may act directly upon these primary sensory neurons. We postulate that NPF could act as an end effector of a temporal control module that may suppress PAIN channel activity or render PAIN neurons incompetent in the sensation of aversive stimuli.

In mammalian models, NPY and its two receptors, Y1 and Y2, have been implicated in the suppression of fear and anxiety (see Heilig, M., 2004; Greco, B. & Carli, M. 2006 which are hereby incorporated herein by reference), and most published data suggest that NPY has an anti-nociceptive role (see Brumovsky, P., et al., 2007, and Tatemoto, K., 2004), which are hereby incorporated herein by reference). For example, NPY injected into the forebrain of rats significantly elevated the nociceptive threshold in a paw-withdraw test (see Li, J.-J., 2005), which is hereby incorporated herein by reference). NPY Y1 receptor knockout mice develop hyperalgesia to acute thermal and chemical pain, and exhibit mechanical hypersensitivity (see Naveilhan, P., et al., 2001), which is hereby incorporated herein by reference). In addition, Y1 and nociceptive TRPV1 channel protein are both strongly expressed in the nociceptors of the DRG, further supporting an inhibitory role of Y1 in TRP family protein-mediated pain sensitivity (see Gibbs, J. et al., 2004), which is hereby incorporated herein by reference). It is conceivable that the action of NPF on peripheral PAIN neurons may be parallel to that of NPY on the DRG neurons. Thus, the present data suggest that the NPF system may be a useful platform for elucidating the molecular and cellular underpinnings of neuropeptide-mediated pain suppression.

### Example 2

### A G-Protein Coupled Neuropeptide Y-Like Receptor Suppresses Behavioral and Sensory Response to Multiple Stressful Stimuli in Drosophila

### Introduction

As discussed above, human NPY and Drosophila Neuropeptide F (NPF) display parallel activities in the role of the organism's response to various stressors. However, it was unclear how NPY family peptides modulate physical and emotional responses to such stressors. The present example demonstrates that NPFR1, a G-protein coupled NPF receptor, exerts an inhibitory effect on larval aversion to diverse stressful stimuli mediated by different subtypes of fly and mammalian TRP family channels. Imaging analysis in larval sensory neurons and cultured human cells showed that NPFR1 attenuates Ca²⁺ influx mediated by fly TRPA and rat TRPV1 channels. These findings suggest that suppression of TRP channel-mediated neural excitation by the conserved NPF/NPFR1 system represents a major mechanism for attaining its broad anti-stress function.

As demonstrated above, the NPF system represents a central regulator of stress response. In food-deprived larvae, NPF signaling is responsible for resistance to diverse stressors, enabling animals to engage in hunger-driven behaviors such as risk-prone food acquisition and motivated procurement of hard food media. Moreover, increased NPF signaling in fed animals selectively elicits stress-resistant seeking behaviors but not food ingestion per se, suggesting that the NPF pathway promotes foraging motivation in hungry animals through an uncharacterized anti-stress mechanism.

As discussed, the transient receptor potential (TRP) family cation channels are evolutionarily conserved sensors of diverse stressful stimuli, and mammalian TRPV1 responds to noxious heat, protons and capsaicin, and plays a prominent role in nociception. TRPV1 activity is regulated by multiple signaling pathways. For example, endogenous pain suppressors such as opioid peptides and endocannabinoids attenuate TRPV1-mediated external noxious stimulation through their G-protein coupled receptors expressed in peripheral nociceptors. On the other hand, TRPV1 activity can also be sensitized by diverse signaling molecules and kinase-mediated pathways, many of which are responsible for inflammatory and neuropathic pain.

As demonstrated in the previous example, the Drosophila PAIN-mediated sugar aversion drives postfeeding larvae out of the aquatic feeding habitat to food-free sites (e.g., from fallen overripe fruits to soil underneath), thereby preventing mobile pupae from microbial killing and drowning in liquid food. However, the PAIN-mediated neuronal pathway for sugar avoidance must be suppressed in younger feeding larvae that live mostly inside sugar-rich food proper. Drosophila larvae appear to be a useful model for investigating the potential functional interaction between NPY family peptides and TRP family channels. As demonstrated below, Drosophila adult flies may also represent a useful model in some circumstances.

The present example demonstrates that NPFR1, a G-protein coupled NPF receptor, is capable of suppressing fly behavioral responses to diverse stressors mediated by different subtypes of TRP family channels. Imaging analysis showed that NPFR1 attenuates Ca2+ influx mediated by fly TRPA in larval sensory neurons and rat TRPV1 channels in cultured human cells. Given the broad distribution of different TRP channel proteins in the central and peripheral neurons, these findings suggest that suppression of TRP channels-mediated neural excitation by NPF/NPFR1 signaling may be a major mechanism for attaining its broad anti-stress function.

### Materials and Methods

Flies, media and larval growth. Conditions for rearing adult flies and egg collection were as described in the following, which are hereby incorporated by reference in their entirety. (Shen and Cai, 2001; Wen et al., 2005). The larvae were raised at 25° C. with exposure to natural lighting. Synchronized eggs were collected within a 2 h interval, and late second instars were transferred to a fresh apple-juice plate with yeast paste (<80 larvae per plate). The pain^{gal4}, UAS-TRPV1, UAS-npfr1^{cDNA}, UAS-npfr1^{dsRNA}, UAS-npf and UAS-PKAc lines are described in the following references, which are incorporated herein by reference in their entirety. (Kiger et al., 1999; Tracey et al., 2003; Wu et al., 2003; Wen et al., 2005; Marella et al., 2006).

Behavioral assays. The migration assay on soft agar media was performed as described previously (Xu et al., 2008), which is hereby incorporated by reference herein in its entirety. Twenty-five postfeeding larvae (96 h AEL) in one plate were allowed to move freely on the medium, and those that crawled onto the plastic surface became less mobile, and eventually formed pupae there. The percentage of pupae on agar media was scored after 24 hours. The clumping assay was also described in the following, which is hereby incorporated by reference herein in its entirety (Wu et al., 2003). Briefly, 45 mm petri dishes containing solid fructose agar (3% agar in a 10% fructose solution) were coated with a thin layer of yeast paste (0.5 g yeast powder in 10% fructose solution). Thirty larvae per plate were allowed to browse for 30 min, and those in clumps were scored immediately. The social burrowing assay was performed on solid agar media containing apple juice or 10% fructose, as described (Wu et al., 2003). All assays, unless stated otherwise, were performed at room temperature in the dark. At least three separate trials were performed per assay.

The thermonociception assay was performed according to previously published procedure with modifications (Tracey et al., 2003), which is hereby incorporated by reference herein in its entirety. The temperature of the electric heating probe was set at 40° C. using a variable transformer (Model 72-110, Tenma), and monitored by a digital thermometer (Model 52 II, Fluke). At least 150 larvae (96 h AEL) were individually tested for each line.

The two-choice preference test was based on a published procedure with some modifications (Al-Anzi et al., 2006), which is hereby incorporated by reference herein in its entirety. 2-day-old males withheld from food for 24 hr were presented with a 48-well plate containing two-colored food media in the alternating well rows. They contain 1% agar/10% fructose with 0.4 mM benzyl-isothiocyanate (BITC) in 75% ethanol or ethanol only. In each assay, 90-100 flies per line were tested in the dark. A preference index was defined as the fraction of larvae choosing the BITC medium, minus the fraction of larvae choosing the BITC-free medium. A preference index close to +1 indicates that the larvae are attracted to BITC, whereas −1 indicates strong rejection. At least three trials were done for each line.

Immunohistochemistry. Larval epidermis was filleted from the dorsal side. Tissues were fixed for 35 min with 4% paraformaldehyde, washed in PBS with 0.4% TritonX-100, and permeablized with Proteinase K digestion followed by post-fixation. Tissues blocked in 10% BSA were then incubated overnight at 4° C. with affinity-purified rabbit anti-NPFR1 antibodies (1:100). The NPFR1 peptide antibodies were raised and purified using two peptide antigens (CMTGHHEGGLRSAIT (SEQ ID NO: 9) and SSNSVRYLDDRHPLC (SEQ ID NO: 10). Alexa 488-conjugated anti-rabbit IgG secondary antibody was diluted to 1:2,000. At least 15 epidermal tissues were examined.

In vivo calcium imaging. Detailed procedures for calcium imaging of sensory neurons and SOARS (Statistical Optimization for the Analysis of Ratiometric Signals) analysis were described previously (Xu et al., 2008). The SOARS method extracts the anti-correlated change of Fluo-4 and Fura-Red signals in a cell (represented by a cluster of ˜100 pixels) that respond to stimulations in a common dynamic pattern. At least 6 epidermal tissues were imaged for each group. To quantify the significance of anti-correlated FRET response to fructose stimulation, p-values were calculated for a periodic (sinusoidal) response at the stimulus frequency using multitaper harmonic analysis, a common method for the detection of sinusoids in noisy data (Thomson, 1982), which is hereby incorporated by reference herein in its entirety.

Transfection and Calcium imaging of HEK 293 cells. HEK293 cells were maintained under standard conditions (37° C., 5% CO2) in Dulbecco's Modified Eagle's Medium (Mediatech) supplemented with 10% fetal bovine serum, penicillin and streptomycin. pcDNA3.1D directional expression system (Invitrogen) was used to clone and express rat TRPV1 and fly NPFR1 cDNA in HEK293 cells. TRPV1 cDNA was PCR amplified from TRPV1 (E600K) sequence (Marella et al., 2006), using the following primers: 5′ CACCATGGAAC AACGGGCTAGCTTA 3′ (SEQ ID NO: 11) and 5′ TTCTTTCTCCCCTGGGACCATGGA 3′ (SEQ ID NO: 12). NPFR1 cDNA was PCR amplified from an NPFR1 cDNA plasmid(34), using two primers: 5′ CACCATGATAATCAGCATGAATCAGA 3′ (SEQ ID NO: 13) and 5′ TTACCGCGGCATCAGCTTGGT 3′ (SEQ ID NO: 14). HEK293 cells were cultured on 8-well polyornithine-coated chambered coverglasses (Nunc) for 24 hrs before transfection. A suspension of 200 μl of water containing 0.4 μg plasmid DNA and 0.8 μl of Lipofectamine 2000 (Invitrogen) was used for transfection. The amounts of TRPV1 and NPFR1 cNDA used were 20 ng and 200 ng, respectively. pcDNA3.1 vector DNA was supplemented, when necessary, to ensure the equal amount of total DNA per transfection.

Calcium imaging was performed between 36-42 hours post transfection at 23° C. Cells were loaded with 1 μM Fluo-4 and 2 μM Fura-red (Invitrogen) for 90 min in Hanks' balanced salt solution (HBSS; Gibco), washed once with HBSS, and imaged using a Zeiss Axiovert 200M scope equipped with a Zeiss LSM 510 Meta laser scanning module. Dyes were excited at 488 nm with argon laser. Emission fluorescence was filtered by a BP505-530 and an LP585 filter. Images were collected for 300 s upon capsaicin stimulation at 1 frame s⁻¹, and analyzed with the SOARS analysis. NPF was synthesized by Quality Controlled Biochemicals. NPF and cyclic nucleotide analogs (Sigma) were pre-incubated at 23° C. with cells for 20-25 min before adding capsaicin.

### Results

NPFR1 Suppression of Peripheral Aversive Stimulation

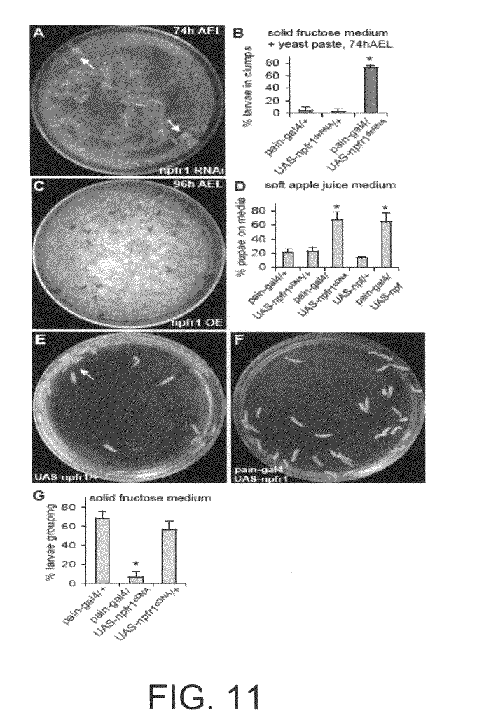

To investigate the potential role of NPFR1 in NPF suppression of premature onset of PAIN-mediated sugar-averse behaviors of feeding larvae, npfr1 activity in the nervous system of feeding larvae (74 h after egg laying, AEL) was knocked down with RNA interference. Like, NPF signaling-deficient larvae, expression of npfr1 double-stranded RNA (dsRNA) in feeding larvae driven by pain-gal4 triggered precocious onset of sugar-elicited grouping behavior normally associated with older postfeeding larvae (FIGS. 11A and 11B). Two PAIN-mediated food-averse behaviors of postfeeding larvae (96 h AEL) that overexpress NPFR1 directed by pain-gal4 were also examined. In soft apple juice agar media, wild type postfeeding larvae normally migrate out the food medium, as shown in the above example. As expected, a majority of control larvae (e.g., UAS-npfr1^{cDNA} alone) moved out of the medium, while about 60% of NPFR1-overexpressing larvae (pain-gal4/UAS-npfr1^{cDNA}) remained (FIGS. 11C and 11D). Moreover, postfeeding larvae expressing NPF (pain-gal4/UAS-npf) also showed attenuated food aversion. Thus, increased NPF or NPFR1 activity dominantly suppresses larval food-averse migration.

When exposed to hard sugar-containing media, postfeeding larvae (96 h AEL), but not feeding larvae, rapidly swarm towards each other and form stable aggregates (Wu et al., 2003). This instinctive cooperative behavior may enable larvae to quickly burrow through sugar-containing hard media (e.g., fruit juice-stained compact surface soil) for pupation (Thomas, 1995; Alyokhin et al., 2001). The present data also demonstrate that pain-gal4/UAS-npfr1^{cDNA} postfeeding larvae failed to display aggregation and burrowing on the 10% fructose agar medium (FIGS. 11E-G). Together, these findings suggest that the G-protein coupled NPF receptor NPFR1 negatively regulates PAIN-mediated larval food-averse behaviors.

NPFR1 Suppression of TRPA Channel-Mediated Nociception

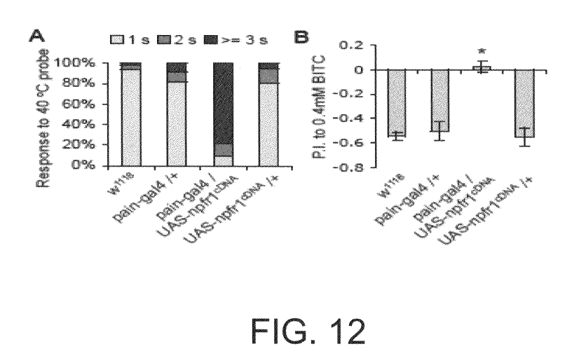

In pain-gal4/UAS-npfr1^{cDNA} larvae, NPFR1 is ectopically expressed in the entire set of PAIN neurons including those that are responsive to noxious heat (Tracey et al., 2003). The effect of NPFR1 on noxious heat response of those larvae was also tested. pain-gal4/UASnpfr1^{cDNA} larvae showed significantly delayed aversive response to the touch of a 40° C. probe (FIG. 12A). In addition, the PAIN-mediated response to isothiocyanate, the pungent ingredient of horseradish, was also abolished by NPFR1 overexpression in adult PAIN neurons (FIG. 12B). These results demonstrate that NPFR1 is capable of suppressing fly sensory responses to various chemical stressors and noxious heat mediated by nociceptive TRPA channels.

Suppression of Mammalian TRPV1-Mediated Avoidance by NPFR1

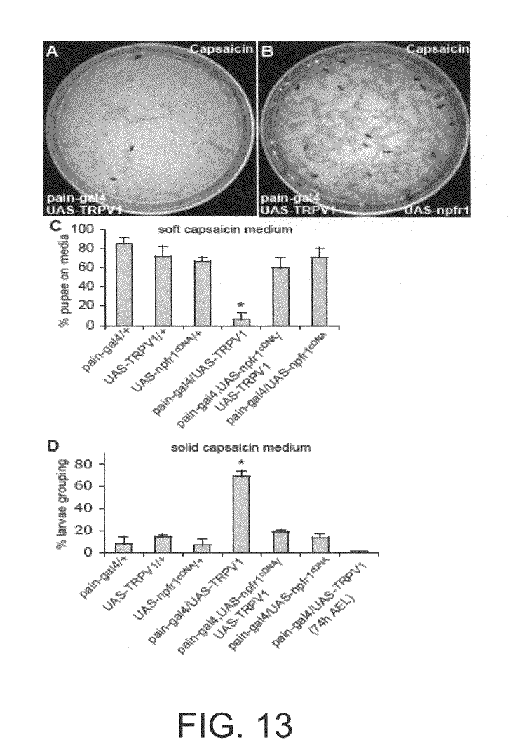

Wild type Drosophila larvae display neither attractive nor aversive response to capsaicin, the spicy substance from hot chili peppers. However, expression of a rat capsaicin receptor TRPV1 in PAIN neurons of postfeeding larvae is sufficient to trigger larval aversion to capsaicin, as demonstrated in the above example. This finding provides an opportunity to test whether NPFR1 is capable of suppressing a mammalian nociceptive TRP channel of a different subtype in Drosophila sensory neurons. The results show that postfeeding larvae co-expressing NPFR1 and TRPV1, driven by pain-gal4, failed to display capsaicin-averse behaviors (FIG. 13). Consistent with this finding, younger feeding larvae expressing rat TRPV1 are also insensitive to capsaicin (see Example 1). These results suggest that NPFR1 signaling is capable of suppressing sensory response to diverse forms of stressors mediated by different subtypes of TRP channels, and its antinociceptive activity may be mediated by a signaling mechanism conserved between flies and mammals.

NPFR1 Suppression of TRPA Channel-Mediated Ca2+ Influx

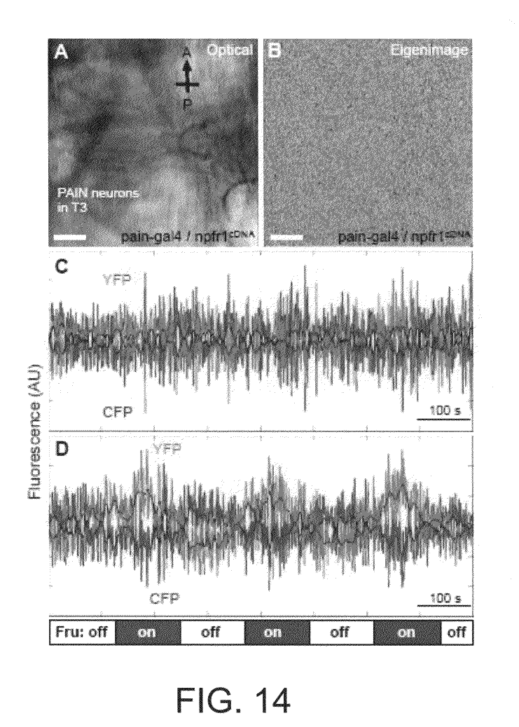

Cellular imaging analysis was also performed to examine how NPFR1 might affect the activity of TRP family channels in sugar-responsive PAIN neurons excitation with a Ca²⁺ indicator, yellow cameleon 2.1 (YC2.1) (see Miyawaki et al., 1999, which is incorporated by reference herein in its entirety). The thoracic PAIN neurons of 96 h AEL postfeeding larvae were imaged using confocal laser scanning microscopy, and subsequently processed using the SOARS algorithm, which is designed to extract the anti-correlated changes between yellow and cyan fluorescence levels in response to fructose stimulation (see Broder et al., 2007, which is hereby incorporated by reference herein in its entirety). Similar to the situation in pain³ larvae (96 h AEL), fructose failed to trigger excitation of the thoracic PAIN neurons in pain-gal4/UAS-npfr1^{cDNA} larvae (96 h AEL, FIG. 14) (see also Example 1). This finding suggests that NPFR1 may function as a potent inhibitor of the activities of TRP channels.

NPFR1 Suppression of TRP Channels in Human Cells

HEK 293 cells have been widely used for studying the suppression of TRPV1 activity by mammalian opioid receptors (see Diaz-Laviada and Ruiz-Llorente, 2005; Vetter et al., 2008, both of which are incorporated by reference herein in their entirety).

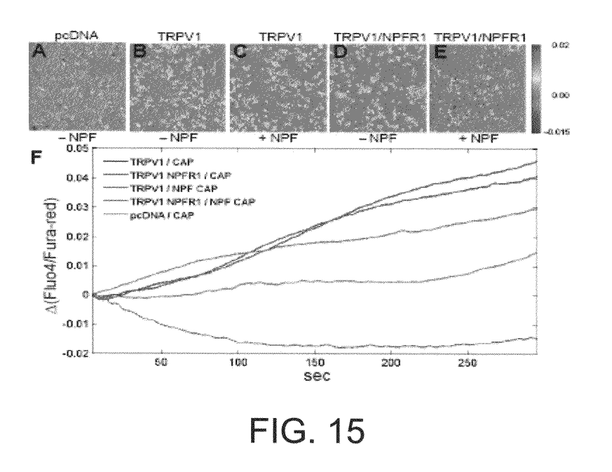

To provide direct evidence that NPF/NPFR1 signaling suppresses TRP channels through a conserved antinociceptive mechanism, NPFR1 signaling was tested to determine sufficiency to suppress TRPV1 in human cells. Both npfr1 and rat TRPV1 cDNAs were co-expressed in HEK 293 cells, and capsaicin-induced Ca²⁺ influx was imaged using Fluo-4 and Fura-red fluorescent dyes. HEK293 cells, co-transfected with NPFR1 and rat TRPV1 cDNAs, displayed significantly reduced TRPV1-mediated Ca²⁺ influx relative to control groups during a 300-sec test period (FIGS. 15A-15F). For example, HEK293 cells transfected with TRPV1 cDNA alone showed significant Ca²⁺ influx in response to capsaicin within 30 seconds, and TRPV1 channels remained active during the entire test period. Experimental cells co-transfected with both NPFR1 and TRPV1 cDNAs, in the presence of NPF, showed drastically attenuated and delayed responses to capsaicin. During the initial 200-second period, cells showed low levels of calcium-dependent fluorescence, and a subset of cells displayed an increase of intracellular Ca²⁺ between 200-300 seconds (FIGS. 15E, and 15F). Addition of NPF to cells transfected with TRPV1 cDNA alone caused a mild reduction in TRPV1 activity (FIG. 55F). Since HEK293 cells express endogenous NPY receptor subtypes (Y2 and Y5, unpublished data), this mild inhibitory effect of NPF might be due to its crossactivation of an endogenous NPY receptor. The present results demonstrate again that NPFR1 suppression of nociceptive TRPV1 activity is mediated by a signaling mechanism conserved between flies and humans.

Modulation of TRPV1 by Cyclic Nucleotides

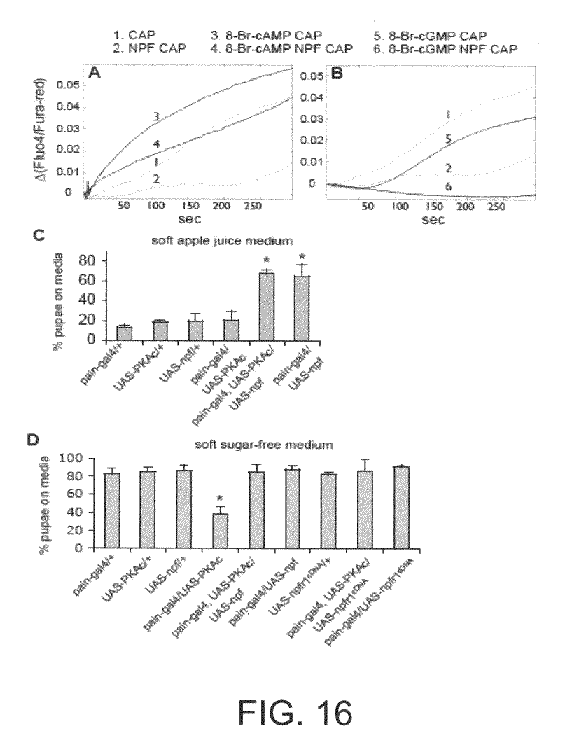

It has been shown that the cAMP/PKA pathway potentiates TRPV1 activity in HEK 293 cells and nociceptive sensory neurons, and may be acutely involved in inflammatory and neuropathic pain (Bhave et al., 2002). In the present study, introduction of a cAMP analog (8-Br-cAMP) to HEK 293 cells significantly increased TRPV1-mediated Ca²⁺ influx (FIG. 16A). Using this sensitized in vitro model, it was found that NPFR1 was capable of attenuating the potentiation of TRPV1 by 8-Br-cAMP, providing further evidence that fly NPFR1 functions well in heterologous mammal cells. In addition, 8-Br-cGMP, a cGMP analog, slightly reduced TRPV1 activity, and NPFR1 suppression of TRPV1 was significantly enhanced in the presence of 8-Br-cGMP (FIG. 16B). Pharmacological evidence suggests that NPFR1 is coupled with Gi/o protein (Garczynski et al., 2002). Therefore, it is possible that the antinociceptive NPFR1 may involve downregulation of intracellular cAMP though inhibition of adenylyl cyclase.

NPF/NPFR1 Suppression of PKA-Induced Hypersensitivity

The in vitro finding raised the question of whether PKA has a sensitizing effect on PAIN-mediated larval sugar aversion. Indeed, postfeeding larvae expressing a constitutively active form of PKA (UAS-PKAc), driven by pain-gal4, showed aversive response to agar media regardless of the presence or absence of sugar (FIGS. 16C and 16D). For example, a majority of pain-gal4/UAS-PKAc larvae migrated out of the sugar-free soft medium, while control larvae (e.g., UAS-PKAc alone) pupated mostly on the medium. These results indicate that the increased activity of the cAMP/PKA pathway causes sensitized behavioral response to media. Mammalian PKA has been shown to sensitize TRPV1 channels and acutely mediates hyperexcitation of nociceptive sensory neurons (Hu et al., 2001; Song et al., 2006). It is possible that fly PKA may have similar excitatory effects in PAIN neurons.

Further tests were conducted to determine whether the NPF pathway is able to suppress sugar aversion of PKA-sensitized larvae. Postfeeding larvae co-expressing UAS-PKAc and UAS-npf, directed by pain-gal4, were placed onto apple juice and sugar-free soft agar media. Most of the larvae pupated on both media (FIGS. 16C and 16D). Moreover, larvae co-expressing UAS-PKAc and UAS-npfr1^{cDNA} also displayed similar phenotype (FIG. 16D). These results indicate that the NPF pathway has a dominant suppressive effect on the sensitization of PAIN neurons by exuberant PKA activity. Since the constitutively active PKAc is thought to be insensitive to the reduction of intracellular cAMP (Kiger et al., 1999), the Gi/o-protein coupled NPF receptor may antagonize the PKAc effect through a cAMP-independent pathway.

### Discussion

The present example demonstrates that the Drosophila NPY-like receptor NPFR1 negatively regulates fly avoidance response to diverse external stressors mediated by different subtypes of TRP family channels. These findings suggest that NPFR1 appears to exert its suppressive effect through attenuation of TRP channel induced neuronal excitation. The present study also provides the first evidence that the antinociceptive functions of invertebrates and mammals can be mediated by a conserved mechanism that suppresses nociceptive TRP family channels. Given the implicated role of human NPY in stress and pain resiliency (Bannon et al., 2000; Thorsell et al., 2000; Thorsell and Heilig, 2002; Zhou et al., 2008), the Drosophila NPY-like system appears to be a promising model for the identification and characterization of genetic factors that influence pain threshold and tolerance and genetic predispositions to pain disorders.

The G-protein coupled receptors of mammalian opioid peptides and endocannabinoids are expressed in peripheral nociceptors, and mediate suppression of peripheral noxious stimulation (Pertwee, 2001; Endres-Becker et al., 2007). The present example demonstrates that the Drosophila NPY-like system suppresses peripheral stressful stimulation in feeding larvae. Several lines of evidence suggest that NPF directly acts on sensory neurons expressing TRPA channel protein PAIN. First, the G-protein coupled NPF receptor NPFR1 is expressed selectively in a subset of PAIN sensory neurons responsive to aversive sugar stimulation. Second, laser ablation of NPFR1-expressing PAIN neurons abolished larval aversion to sugar. Finally, overexpression of NPFR1 in PAIN neurons blocks sugar-stimulated TRPA channel activity. The mammalian NPY receptor Y1 is also expressed in the primary nociceptive neurons of dorsal root ganglia (DRG) and trigeminal ganglia (Brumovsky et al., 2007). The pharmacological study with an Y1 agonist supports a role of Y1 in the reduction of capsaicin-stimulated release of calcitonin gene-related peptide (Gibbs and Hargreaves, 2008). Together, these findings suggest that NPY family peptides are involved in the modulation of the sensation of external stressful stimuli in flies and possibly mammals.

TRPV1 appears to be one of the primary targets of endogenous antinociceptive activities in mammals. TRPV1 and the receptors of opioid peptides, endocannabinoids and NPY co-localize in different nociceptors. It has also been shown that both opioid and cannabinoid receptors suppress TRPV1-mediated Ca²⁺ influx in primary sensory neurons or in HEK 293 cells (Endres-Becker J, et al., 2007; Vetter I et al., 2008). Now evidence has been obtained that activation of NPFR1 also suppresses TRP channel activities in larval sensory neurons and heterologous HEK293 cells. These findings suggest that a conserved signaling mechanism may underlie the suppression of peripheral stressful stimulation by invertebrate and mammalian antinociceptive activities. It remains largely unclear how NPFR1 and mammalian opioid and cannabinoid receptors negatively regulate TRPV1 activity. Since all of these receptors are coupled with Gi/o, their antinociceptive activities may be mediated by a common mechanism involving downregulation of adenylyl cyclase and intracellular cAMP. Expression of mammalian TRPV1 renders the transgenic larvae to display migratory and grouping behaviors in response to aversive capsaicin stimulation. However, increased PKA activity in the PAIN neurons of postfeeding larvae is sufficient to elicit such behaviors without the need of any aversive chemicals in the medium. Thus, higher PKA activity appears to sensitize larval nociceptive sensory neurons. Consistent with this notion, PKA has been shown to potentiate TRP channel activity through direct phosphorylation the N-terminal domain (Bhave et al., 2002). PKA activity can also reverse the desensitized TRP channels. In the DRG sensory neurons of rats with spinal nerve ligation, PKA activity is acutely required for the peripheral neuropathic pain (Hu et al., 2001). The PKA-elicited independence of aversive stimulation may result from its sensitization of an endogenous TRP channel (e.g., PAIN).

It remains to be determined how NPFR1 signaling dominantly suppresses the migratory and grouping behaviors in wild type and PKA-overexpressing larvae. One simple explanation is that NPFR1 signaling may lead to a large reduction of the intracellular cAMP level, thereby downregulating the activity of PKA. However, this explanation may not be completely satisfactory because the transgenic UAS-PKAc construct encodes a constitutively active form of PKA whose activity is cAMP-independent. Therefore, the present findings strongly argue for the existence of PKA-independent mechanism(s) by which NPFR1 suppresses TRP channel in PKA-overexpressing PAIN neurons.

NPY family peptides have been shown to promote diverse stress-resistant behaviors. Over expression of NPFR1 in flies and administration of NPY in mice rendered animals to be more willing to work for food and become more resilient to aversive taste and deleteriously cold temperature (Flood and Morley, 1991; Jewett et al., 1995; Wu et al., 2005a; Wu et al., 2005b; Lingo et al., 2007). The present findings of suppression of different TRP family channels by a single receptor NPFR1 provides a molecular explanation of how a NPY family peptide could mediate resiliency to diverse gustatory, thermal and mechanical stressors. In food-deprived larvae, reduced fly insulin signaling triggers NPFR1-mediated stressor-resistant foraging activities (Wu et al., 2005a; Wu et al., 2005b). The present findings also raise the possibility that TRP family channels may be indirectly regulated by insulin signaling.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, or ±10%, of the numerical value(s) being modified. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

### REFERENCES

Al-Anzi B, Tracey W D, Jr., Benzer S (2006) Response of Drosophila to wasabi is mediated by painless, the fly homolog of mammalian TRPA1/ANKTM1. Curr Biol 16:1034-1040.
Alyokhin A, Mille C, Messing R, Duan J (2001) Selection of Pupation Habitats by Oriental Fruit Fly Larvae in the Laboratory. Journal of Insect Behavior 14:57-67.
Ashburner M (1989) Drosophila: A Laboratory Handbook. Cold Spring Harbor, N.Y.: Cold Spring Harbor Laboratory.
Bannon A W, Seda J, Carmouche M, Francis J M, Norman M H, Karbon B, McCaleb M L (2000) Behavioral characterization of neuropeptide Y knockout mice. Brain Research 868:79-87.
Bhave G, Zhu W, Wang H, Brasier D J, Oxford G S, Gereau RWt (2002) cAMP-dependent protein kinase regulates desensitization of the capsaicin receptor (VR1) by direct phosphorylation. Neuron 35:721-731.
Bolhuis, J. J. & Gahr, M. Neural mechanisms of birdsong memory. Nat Rev Neurosci 7, 347-357 (2006).
Broder J, Majumder A, Porter E, Srinivasamoorthy G, Keith C, Lauderdale J, Sornborger A (2007) Estimating weak ratiometric signals in imaging data. I. Dual-channel data. J Opt Soc Am A Opt Image Sci V is 24:2921-2931.
Brown, M. R., et al. Identification of a Drosophila brain-gut peptide related to the neuropeptide Y family. Peptides 20, 1035-1042 (1999).
Brumovsky, P., Shi, T. S., Landry, M., Villar, M. J. & Hokfelt, T. Neuropeptide tyrosine and pain. Trends in pharmacological sciences 28, 93-102 (2007).
Caterina M J, Julius D (2001) The vanilloid receptor: a molecular gateway to the pain pathway. Annu Rev Neurosci 24:487-517.
Caterina M J, Schumacher M A, Tominaga M, Rosen T A, Levine J D, Julius D (1997) The capsaicin receptor: a heat-activated ion channel in the pain pathway. Nature 389:816-824.
Chiang H C H, A. G. (1950) An analytical study of population growth in Drosophila melanogaster. Ecol Monogr 20:172-206.
Cohen, B., Wimmer, E. A. & Cohen, S. M. Early development of leg and wing primordia in the Drosophila embryo. Mech Dev 33, 229-240 (1991).
Diaz-Laviada I, Ruiz-Llorente L (2005) Signal transduction activated by cannabinoid receptors. Mini Rev Med Chem 5:619-630.
Endres-Becker J, Heppenstall P A, Mousa S A, Labuz D, Oksche A, Schafer M, Stein C, Zollner C (2007) Mu-opioid receptor activation modulates transient receptor potential vanilloid 1 (TRPV1) currents in sensory neurons in a model of inflammatory pain. Mol Pharmacol 71:12-18.
Fan, X., et al. New statistical methods enhance imaging of cameleon fluorescence resonance energy transfer in cultured zebrafish spinal neurons. J Biomed Opt 12, 034017 (2007).
Garczynski S F, Brown M R, Shen P, Murray T F, Crim J W (2002) Characterization of a functional neuropeptide F receptor from Drosophila melanogaster. Peptides 23:773-780.
Gibbs, J., Flores, C. M. & Hargreaves, K. M. Neuropeptide Y inhibits capsaicin-sensitive nociceptors via a Y1-receptor-mediated mechanism. Neuroscience 125, 703-709 (2004).
Greco, B. & Carli, M. Reduced attention and increased impulsivity in mice lacking NPY Y2 receptors: relation to anxiolytic-like phenotype. Behav Brain Res 169, 325-334 (2006).
Han, D. D., Stein, D., and Stevens, L. M. Investigating the function of follicular subpopulations during Drosophila oogenesis through hormone-dependent enhancer-targeted cell ablation. Development 127, 573-583 (2000)
Heilig, M. The NPY system in stress, anxiety and depression. Neuropeptides 38, 213-224 (2004).
Hu S J, Song X J, Greenquist K W, Zhang J M, LaMotte R H (2001) Protein kinase A modulates spontaneous activity in chronically compressed dorsal root ganglion neurons in the rat. Pain 94:39-46.
Hucho T B, Dina O A, Levine J D (2005) Epac mediates a cAMP-to-PKC signaling in inflammatory pain: an isolectin B4(+) neuron-specific mechanism. J Neurosci 25:6119-6126.
Ji, R. R., Zhang, X., Wiesenfeld-Hallin, Z. & Hokfelt, T. Expression of neuropeptide Y and neuropeptide Y (Y1) receptor mRNA in rat spinal cord and dorsal root ganglia following peripheral tissue inflammation. J Neurosci 14, 6423-6434 (1994).
Kiger J A, Jr., Eklund J L, Younger S H, O'Kane C J (1999) Transgenic inhibitors identify two roles for protein kinase A in Drosophila development. Genetics 152:281-290.
Kitamoto, T. Targeted expression of temperature-sensitive dynamin to study neural mechanisms of complex behavior in Drosophila. J Neurogenet 16, 205-228 (2002).
Larhammar, D. Evolution of neuropeptide Y, peptide YY and pancreatic polypeptide. Regul Pept 62, 1-11 (1996).
Li, J.-J., Zhou, X. & Yu, L.-C. Involvement of neuropeptide Y and Y1 receptor in antinociception in the arcuate nucleus of hypothalamus, an immunohistochemical and pharmacological study in intact rats and rats with inflammation. Pain 118, 232-242 (2005).
Liu, L., Yermolaieva, O., Johnson, W. A., Abboud, F. M. & Welsh, M. J. Identification and function of thermosensory neurons in Drosophila larvae. Nat Neurosci 6, 267-273 (2003).
Macleod, G. T., Hegstrom-Wojtowicz, M., Charlton, M. P. & Atwood, H. L. Fast calcium signals in Drosophila motor neuron terminals. J Neurophysiol 88, 2659-2663 (2002).
Marella S, Fischler W, Kong P, Asgarian S, Rueckert E, Scott K (2006) Imaging taste responses in the fly brain reveals a functional map of taste category and behavior. Neuron 49:285-295.
McVeigh, P., Kimber, M. J., Novozhilova, E. & Day, T. A. Neuropeptide signalling systems in flatworms. Parasitology 131 Suppl, S41-55 (2005).
Mitra, P. P. & Pesaran, B. Analysis of dynamic brain imaging data. Biophys J 76, 691-708 (1999).
Mitra, P. & Bokil, H. Observed brain dynamics (Oxford University Press, New York, 2008).
Miyawaki A, Griesbeck O, Heim R, Tsien R Y (1999) Dynamic and quantitative Ca2+ measurements using improved cameleons. Proc Natl Acad Sci USA 96:2135-2140.
Montell C (2005) Drosophila TRP channels. Pflugers Arch.
Montell C, Birnbaumer L, Flockerzi V (2002) The TRP channels, a remarkably functional family. Cell 108:595-598.
Moran M M, Xu H, Clapham D E (2004) TRP ion channels in the nervous system. Curr Opin Neurobiol 14:362-369.
Mostany R, Diaz A, Valdizan E M, Rodriguez-Munoz M, Garzon J, Hurle M A (2008) Supersensitivity to mu-opioid receptor-mediated inhibition of the adenylyl cyclase pathway involves pertussis toxin-resistant Galpha protein subunits. Neuropharmacology 54:989-997.
Mukhopadhyay S, Shim J Y, Assi A A, Norford D, Howlett A C (2002) CB(1) cannabinoid receptor-G protein association: a possible mechanism for differential signaling. Chem Phys Lipids 121:91-109.
Naveilhan, P., et al. Reduced antinociception and plasma extravasation in mice lacking a neuropeptide Y receptor. Nature 409, 513-517 (2001).
Pertwee R G (2001) Cannabinoid receptors and pain. Prog Neurobiol 63:569-611.
Ramsey I S, Delling M, Clapham D E (2006) An introduction to TRP channels. Annual Review of Physiology 68:619-647.
Roberts. Drosophila: A Practical Approch. (1986).
Scott, K., et al. A chemosensory gene family encoding candidate gustatory and olfactory receptors in Drosophila. Cell 104, 661-673 (2001).
Shen P, Cai H N (2001) Drosophila neuropeptide F mediates integration of chemosensory stimulation and conditioning of the nervous system by food. J Neurobiol 47:16-25.
Sokolowski, M. B. NPY and the regulation of behavioral development. Neuron 39, 6-8 (2003).
Song X-J, Wang Z-B, Gan Q, Walters E T (2006) cAMP and cGMP Contribute to Sensory Neuron Hyperexcitability and Hyperalgesia in Rats With Dorsal Root Ganglia Compression. J Neurophysiol 95:479-492.
Sornborger, A., Sailstad, C., Kaplan, E. & Sirovich, L. Spatiotemporal analysis of optical imaging data. Neuroimage 18, 610-621 (2003).
Tatemoto, K. Neuropeptide Y: History and overview. Handb. exp. pharmacol. 162, 1-21 (2004).
Thomas D (1995) Predation on the soil inhabiting stages of the Mexican fruit fly. Southwest Entomol 20:61-71.
Thomson D (1982) Spectrum estimation and harmonic analysis. Proc IEEE 70:1055-1096.
Thorsell A, Heilig M (2002) Diverse functions of neuropeptide Y revealed using genetically modified animals. Neuropeptides 36:182-193.
Thorsell A, Michalkiewicz M, Dumont Y, Quirion R, Caberlotto L, Rimondini R, Mathe A A, Heilig M (2000) Behavioral insensitivity to restraint stress, absent fear suppression of behavior and impaired spatial learning in transgenic rats with hippocampal neuropeptide Y overexpression. Proc Natl Acad Sci USA 97:12852-12857.
Tobin, D. M., et al. Combinatorial Expression of TRPV Channel Proteins Defines Their Sensory Functions and Subcellular Localization in C. elegans Neurons. Neuron 35, 307-318 (2002).
Tracey W D, Jr., Wilson R I, Laurent G, Benzer S (2003) painless, a Drosophila gene essential for nociception. Cell 113:261-273.
Vetter I, Cheng W, Peiris M, Wyse B D, Roberts-Thomson S J, Zheng J, Monteith G R, Cabot P J (2008) Rapid, opioid-sensitive mechanisms involved in transient receptor potential vanilloid 1 sensitization. J Biol Chem 283:19540-19550.
Wen T, Parrish C A, Xu D, Wu Q, Shen P (2005) Drosophila neuropeptide F and its receptor, NPFR1, define a signaling pathway that acutely modulates alcohol sensitivity. Proc Natl Acad Sci USA 102:2141-2146.
Wu Q, Zhao Z, Shen P (2005a) Regulation of aversion to noxious food by Drosophila neuropeptide Y- and insulin-like systems. Nat Neurosci 8:1350-1355.
Wu Q, Zhang Y, Xu J, Shen P (2005b) Regulation of hunger-driven behaviors by neural ribosomal S6 kinase in Drosophila. Proc Natl Acad Sci USA 102:13289-13294.
Wu Q, Wen T, Lee G, Park J H, Cai H N, Shen P (2003) Developmental control of foraging and social behavior by the Drosophila neuropeptide Y-like system. Neuron 39:147-161.
Xu J, Sornborger A T, Lee J K, Shen P (2008) Drosophila TRPA channel modulates sugar-stimulated neural excitation, avoidance and social response. Nat Neurosci 11:676-682.
Zhou Z et al. (2008) Genetic variation in human NPY expression affects stress response and emotion. Nature 452:997-1001.
Zhu W, Xu P, Cuascut F X, Hall A K, Oxford G S (2007) Activin acutely sensitizes dorsal root ganglion neurons and induces hyperalgesia via PKC-mediated potentiation of transient receptor potential vanilloid I. J Neurosci 27:13770-13780.

SEQUENCES

SEQ ID NO: 1

Drosophila melanogaster
painless (pain) peptide
PRT
Accession # NM_138135
MDFNNCGFIDPQAQLAGALAKQDIRQFVAALDSGALADLQDDRHTSIYEK
ALSTPGCRDFIEACIDHGSQVNYINKKLDKAAISYAADSRDPGNLAALLK
YRPGNKVQVDRKYGQLTPLNSLAKNLTDENAPDVYSCMQLLLDYGASPNI
VDQGEFTPLHHVLRKSKVKAGKKELIQLFLDHPELDIDSYRNGEVRRLLQ
AQFPELKLPEERHTGPEIDIQTLQRTLRDGDETLFEQQFAEYLQNLKGGA
DNQLNAHQEEYFGLLQESIKRGRQRAFDVILSTGMDINSRPGRANEANLV
ETAVIYGNWQALERLLKEPNLRLTPDSKLLNAVIGRLDEPPYDGSSHQRC
FELLINSDRVDINEADSGRLVPLFFAVKYRNTSAMQKLLKNGAYIGSKSA
FGTLPIKDMPPEVLEEHFDSCITTNGERPGDQNFEIIIDYKNLMRQERDS
GLNQLQDEMAPIAFIAESKEMRHLLQHPLISSFLFLKWHRLSVIFYLNFL
IYSLFTASIITYTLLKFHESDQRALTAFFGLLSWLGISYLILRECIQWIM
SPVRYFWSITNIMEVALITLSIFTCMESSFDKETQRVLAVFTILLVSMEF
CLLVGSLPVLSISTHMLMLREVSNSFLKSFTLYSIFVLTFSLCFYILFGK
SVEEDQSKSATPCPPLGKKEGKDEEQGFNTFTKPIEAVIKTIVMLTGEFD
AGSIQFTSIYTYLIFLLFVIFMTIVLFNLLNGLAVSDTQVIKAQAELNGA
ICRTNVLSRYEQVLTGHGRAGFLLGNHLFRSICQRLMNIYPNYLSLRQIS
VLPNDGNKVLIPMSDPFEMRTLKKASFQQLPLSAAVPQKKLLDPPLRLLP
CCCSLLTGKCSQMSGRVVKRALEVIDQKNAAEQRRKQEQINDSRLKLIEY
KLEQLIQLVQDRK

SEQ ID NO: 2

Drosophila melanogaster
DNA, painless (pain)
Gene ID 37985
gtcgttgtct ggatattaac gaggaagacc aaaccaatgg
actttaacaa ctgcggcttc attgatccgc aggcccagct
agctggagct ttggccaagc aggacatccg acagttcgtt
gctgccctgg acagcggtgc cctggccgat ctacaagacg
accgccatac cagtatctac gagaaggcac tctcaacacc
aggttgtcgt gacttcattg aagcctgcat cgaccacggc
agccaggtga actacatcaa caagaagctg gacaaggccg
caatcagcta tgcggctgac tctagggatc caggaaacct
ggcggctctc cttaagtacc gccccggaaa caaagtccag
gttgatagaa aatatgggca gcttactcca cttaactcac
ttgccaagaa tctcacggat gaaaatgccc cagacgtgta
ctcctgcatg caactcttgc tggactacgg cgcctcgccg
aatatcgtag accagggcga gttcacaccc ttgcaccatg
tgctgagaaa gagcaaggtg aaggctggga agaaggaact
gattcagctc tttctggacc atccggagct ggatatcgat
agttaccgaa acggggaggt gcgcagactg ctgcaggcgc
aatttccgga gcttaagctg ccggaagagc gtcataccgg
gccggagatt gacatccaaa ctcttcaaag gactctacgg
gacggggacg aaacactgtt tgagcagcag ttcgctgagt
acttgcagaa tctcaaaggc ggagcggata accaactaaa
tgcccaccag gaggaatact tcggactgct gcaggagagc
atcaagaggg gcaggcagcg agccttcgat gtcattttgt
ccactggcat ggatatcaac tcgagaccag gcagggccaa
cgaggccaat ctcgtagaga cggccgtgat atacggtaac
tggcaggcgt tggagcgact gcttaaggag ccaaacctgc
gacttactcc agactccaag ctactaaatg cagtaatcgg
ccgtctggat gagccaccgt atgatggctc cagccaccag
cgctgctttg aattgctcat taacagcgat cgcgtagaca
tcaacgaagc tgattccgga cgcctggtgc ctctgttctt
cgctgttaag taccgcaaca cgagtgcgat gcaaaaactc
ctgaagaacg gtgcctacat tggttctaag agcgcatttg
gcacactacc catcaaggac atgccacccg aggttctcga
agagcacttc gactcgtgta tcaccacaaa cggagagagg
cctggtgacc agaactttga gatcatcatc gattataaga
acctaatgcg ccaggagaga gactccggac tcaaccagct
gcaagacgaa atggccccga tcgcattcat cgccgagtcg
aaggagatgc gccacctgct ccagcacccg ctgatctcga
gctttctatt cctcaagtgg caccgacttt ccgtgatatt
ctacctgaac ttcctgatat actcgctttt taccgcctcc
ataattacct acacgctcct caagttccac gaaagcgatc
aaagggctct tactgcattt ttcggattgc tttcctggct
gggaatcagc taccttatat tacgggagtg catccagtgg
ataatgtctc cagttcggta cttttggtct ataacgaata
ttatggaggt ggctcttatt acactatcta tctttacctg
catggaatcc agcttcgaca aggagacgca gcgcgtctta
gccgtattta ccatcctact cgtctccatg gagttttgtt
tactagtggg ctccctgcca gtgctctcaa tttcgacgca
catgctgatg ctgcgagagg tgtcaaacag cttcttaaag
agctttaccc tctactcgat cttcgtgctc accttcagcc
tgtgtttcta tatcctcttc ggcaagtcag tggaggaaga
ccagtctaaa agcgctacgc catgtccacc tctggggaag
aaggagggga aggacgagga acagggcttc aacacattta
ccaagcctat cgaggccgtg atcaagacca ttgtgatgct
gacaggcgag tttgacgccg gaagcatcca gtttaccagc
atctacacct acctgatttt cctgctcttc gtgatcttta
tgacgatagt gctgttcaac cttttgaacg gtcttgcagt
gagcgacacc caagttatta aggctcaggc ggaactgaac
ggagccattt gcagaaccaa cgtccttagt cggtacgagc
aggttctcac tggccacgga cgcgctgggt ttttgttggg
caaccatctc ttccgcagca tctgccaacg tttgatgaac
atctacccga actacttaag tctgcgtcag atttccgtgc
tgccgaacga tggaaacaaa gtgcttattc caatgagcga
tcccttcgaa atgaggaccc ttaagaaggc tagctttcag
caattgcccc tgagtgctgc agtgccccag aagaagctgt
tggatccacc gcttagactt ctgccctgct gctgttccct
gctcaccgga aagtgctccc agatgagcgg ccgggtggtc
aaacgggccc tcgaggtaat cgatcagaag aacgcggcgg
agcagaggcg gaaacaggaa cagatcaacg acagtcgact
gaagctgatc gagtacaagc tggagcaatt aatacagctg
gtccaggacc ggaagtgatg gagaatgtat tttggtagct
ttagtattta tgagactaat caacctttta gaacgatttg
catttaacat tcagtttaaa gagccgagtt agtcggaaat
tgtttttatt aacatacgag taatgaaatt gaacaaaacc
cttaataatt gtcagtaagt aagtagtata taatggttat
atagacagta aatattgtat aaacgaatat cattactgta
ctatttgtac ccgagtaaat atttaatttc aaatgtt

SEQ ID NO: 3

Drosophila melanogaster
PRT
Accession # NM_079521
MIISMNQTEPAQLADGEHLSGYASSSNSVRYLDDRHPLDYLDLGTVHALN
TTAINTSDLNETGSRPLDPVLIDRFLSNRAVDSPWYHMLISMYGVLIVFG
ALGNTLVVIAVIRKPIMRTARNLFILNLAISDLLLCLVTMPLTLMEILSK
YWPYGSCSILCKTIAMLQALCIFVSTISITAIAFDRYQVIVYPTRDSLQF
VGAVTILAGIWALALLLASPLFVYKELINTDTPALLQQIGLQDTIPYCIE
DWPSRNGRFYYSIFSLCVQYLVPILIVSVAYFGIYNKLKSRITVVAVQAS
SAQRKVERGRRMKRTNCLLISIAIIFGVSWLPLNFFNLYADMERSPVTQS
MLVRYAICHMIGMSSACSNPLLYGWLNDNFRKEFQELLCRCSDTNVALNG
HTTGCNVQAAARRRRKLGAELSKGELKLLGPGGAQSGTAGGEGGLAATDF
MTGHHEGGLRSAITESVALTDHNPVPSEVTKLMPR

SEQ ID NO: 4

Drosophila melanogaster
DNA
neuropeptide F receptor (NPFR1)
Gene ID 40754
aacagatggt cgctgactgt gcacgcgtgt ggttatcgga
gatcagtaaa cagcccaact aaacaccgaa acttactgta
ataaaaaaaa acgggaaata agcgaaataa tcaaaatgcg
gccgcatact tatttataat tttgaggcgg ccgagcaccg
gggccccaaa ctctttggat ctgcacggaa tccagaattc
cgagagagca aaaacacaaa gcgaagtccc gtgagtgcat
tccaagttga aaactaagtg agcaactgct gctttggcag
ccggaaaaac agagattcac tcgtgtcact cgcagaagga
aaaacaagaa ccgacggcca ggaaaacaat acggtaccac
gcactatagt aaatatatag catacatatc cccagggcga
aggagattgc caggacgatg ataatcagca tgaatcagac
ggagcccgcc cagctggcag atggggagca tctgagtgga
tacgccagca gcagcaacag cgtgcgctat ctggacgacc
ggcatccgct ggactacctt gacctgggca cggtgcacgc
cctcaacacc actgccatca acacctcgga tctgaatgag
actgggagca ggccgctgga cccggtgctt atcgataggt
tcctgagcaa cagggcggtg gacagcccct ggtaccacat
gctcatcagc atgtacggcg tgctaatcgt cttcggcgcc
ctaggcaaca ccctggttgt tatagccgtc atccggaagc
ccatcatgcg cactgctcgc aatctgttca tcctcaacct
ggccatatcg gacctacttt tatgcctagt caccatgccg
ctgaccttga tggagatcct gtccaagtac tggccctacg
gctcctgctc catcctgtgc aaaacgattg ccatgctgca
ggcactttgt attttcgtgt cgacaatatc cataacggcc
attgccttcg acagatatca ggtgatcgtg taccccacgc
gggacagcct gcagttcgtg ggcgcggtga cgatcctggc
ggggatctgg gcactggcac tgctgctggc ctcgccgctg
ttcgtctaca aggagctgat caacacagac acgccggcac
tcctgcagca gatcggcctg caggacacga tcccgtactg
cattgaggac tggccaagtc gcaacgggcg cttctactac
tcgatcttct cgctgtgcgt acaatacctg gtgcccatcc
tgatcgtctc ggtggcatac ttcgggatct acaacaagct
gaagagccgc atcaccgtgg tggctgtgca ggcgtcctcc
gctcagcgga aggtggagcg ggggcggcgg atgaagcgca
ccaactgcct actgatcagc atcgccatca tctttggcgt
ttcttggctg ccgctgaact ttttcaacct gtacgcggac
atggagcgct cgccggtcac tcagagcatg ctagtccgct
acgccatctg ccacatgatc ggcatgagct ccgcctgctc
caacccgttg ctctacggct ggctcaacga caacttccgt
aaagaatttc aagaactgct ctgccgttgc tcagacacta
atgttgctct taacggtcac acgacaggct gcaacgtcca
ggcggcggcg cgcaggcgtc gcaagttggg cgccgaactc
tccaaaggcg aactcaagct gctggggcca ggcggcgccc
agagcggtac cgccggcggg gaaggcggtc tggcggccac
cgacttcatg accggccacc acgagggcgg actgcgcagc
gccataaccg agtcggtggc cctcacggac cacaaccccg
tgccctcgga ggtcaccaag ctgatgccgc ggtaaagcac
agggtagtcc taaggtcctt gaggtctggt ctcgtgtcta
agtcctcatg atacacgcgt gcatgtcctt ttgtacgccc
tcgggctgat tggatttgca tgctccaaac gtcgctgctg
ctcgctttac gtttcacttg ttccaactgc aactgccacc
tctctagaac actgagcgaa atgccgtgtc ctctaatcgg
gaaacactct ggctgtaaaa tctataagca gccgagtcaa
acgtttctag cgttctaaaa gtttcttatg attattttat
tttatatatt aataacaatg acttcgttcc caattatatg
cttgttttca tcgtttttga atgtaacaat tgatcaatat
tcgaccaaaa gcaagttttg aaatatttgt gtaaatatcg
ttttcaaatt tgttcgcctt aatcttactt aataataata
ataataataa tctttactcc gataatcatt aacgtaacat
ttctacttgt aaaaatattt ctgatctaag gggcttgctc
ttttcggctc caccttgaat tacttttcag ttgactaact
aggcgtatat ttttgtcagt gtatgcatgc gctcctctta
tcgttgcctt gtcgagctgt aactcttgtt gttgccatgt
tgtgacatta tttgcttttg agctgcaatc gattatgacc
cgtcttttgt gacacatttt cagtttggaa cagtactaaa
ttggcaatca atcctggagc agcaggcggc tggagcagca
ttctagggag gcgactgcct gtcacccata gacttaacac
gtattgtcca gtgatccaaa gccaagctga gttagcccta
agttaagaca cacgcgacta agagctcgaa gcctgtaaac
tattcttaaa cgaccatgtc atggcatcca tcatcaactc
ggactaagtc tatggttaga tcactttcgt atcaaatgcc
gaaaagtaat tgaatgagcc cccaattaga tttcgggtat
ttgataagtc gaatacgcct aagactcgaa taagttctct
caacagttcc taggaaatat tttcgtttta tctcagcatt
tcttggtatc atctaagcta aggattagct ataattgatg
ttctgttcgc tattcaataa tgataggata ggtcgaagtc
cactaagcca aagtgaattt aaagtatagt atagtataaa
gtataattgt aaaagataac tttaaaataa atgtcagctg
ctcttaaaca tttaatgcaa

SEQ ID NO: 5

Drosophila melanogaster
PRT
Accesssion # AAF55339
Drosophila NPF peptide
MCQTMRCILV ACVALALLAA GCRVEASNSR PPRKNDVNTM
ADAYKFLQDL DTYYGDRARV RFGKRGSLMD ILRNHEMDNI
NLGKNANNGG EFARGFNEEE IF

SEQ ID NO: 6

Drosophila melanogaster
DNA
Gene ID: 42018
DNA encoding for Drosophila NPF peptide
tacagtccga cgaacaattg cattgtgaca ccgttgcgct
ttccaatact caaactccca gttgaaccag aactatgtgc
caaacaatgc gttgcatcct ggttgcctgt gtggcccttg
ccctcctagc cgccggctgc cgagtggagg cgtccaactc
cagacctccg cgaaagaacg atgtcaacac tatggctgat
gcctacaagt tcctgcagga tctggacacc tactacggcg
acagagcccg cgttcggttc ggaaagcgcg gatcgctgat
ggatatcctg aggaatcacg agatggacaa cataaatcta
ggaaaaaatg ccaacaatgg aggagaattt gctcgcggtt
ttaatgagga ggagatattc taaatccatt ttagacgacc
atggcaacgt cactaactca tgatgatagt tattagcata
cgcattttta tttaaattgt ttttcggggc aatagtttaa
cgtgctggga aagaacaagt agttgcagct acagaaataa
gtatttactc tagtcttgat gtcggttgaa taaatgaatt
accccaataa

SEQ ID NO: 7

Rattus norvegicus
PRT
Accession # NM_031982
transient receptor potential cation channel, sub-
family V, member 1 (Trpv1)
MEQRASLDSEESESPPQENSCLDPPDRDPNCKPPPVKPHIFTTRSRTRLF
GKGDSEEASPLDCPYEEGGLASCPIITVSSVLTIQRPGDGPASVRPSSQD
SVSAGEKPPRLYDRRSIFDAVAQSNCQELESLLPFLQRSKKRLTDSEFKD
PETGKTCLLKAMLNLHNGQNDTIALLLDVARKTDSLKQFVNASYTDSYYK
GQTALHIAIERRNMTLVTLLVENGADVQAAANGDFFKKTKGRPGFYFGEL
PLSLAACTNQLAIVKFLLQNSWQPADISARDSVGNTVLHALVEVADNTVD
NTKFVTSMYNEILILGAKLHPTLKLEEITNRKGLTPLALAASSGKIGVLA
YILQREIHEPECRHLSRKFTEWAYGPVHSSLYDLSCIDTCEKNSVLEVIA
YSSSETPNRHDMLLVEPLNRLLQDKWDRFVKRIFYFNFFVYCLYMIIFTA
AAYYRPVEGLPPYKLKNTVGDYFRVTGEILSVSGGVYFFFRGIQYFLQRR
PSLKSLFVDSYSEILFFVQSLFMLVSVVLYFSQRKEYVASMVFSLAMGWT
NMLYYTRGFQQMGIYAVMIEKMILRDLCRFMFVYLVFLFGFSTAVVTLIE
DGKNNSLPMESTPHKCRGSACKPGNSYNSLYSTCLELFKFTIGMGDLEFT
ENYDFKAVFIILLLAYVILTYILLLNMLIALMGETVNKIAQESKNIWKLQ
RAITILDTEKSFLKCMRKAFRSGKLLQVGFTPDGKDDYRWCFRVDEVNWT
TWNTNVGIINEDPGNCEGVKRTLSFSLRSGRVSGRNWKNFALVPLLRDAS
TRDRHATQQEEVQLKHYTGSLKPEDAEVFKDSMVPGEK

SEQ ID NO: 8

DNA
Rattus norvegicus
Gene ID 83810
cagctccaag gcacttgctc catttggggt gtgcctgcac
ctagctggtt gcaaattggg ccacagagga tctggaaagg
atggaacaac gggctagctt agactcagag gagtctgagt
ccccacccca agagaactcc tgcctggacc ctccagacag
agaccctaac tgcaagccac ctccagtcaa gccccacatc
ttcactacca ggagtcgtac ccggcttttt gggaagggtg
actcggagga ggcctctccc ctggactgcc cttatgagga
aggcgggctg gcttcctgcc ctatcatcac tgtcagctct
gttctaacta tccagaggcc tggggatgga cctgccagtg
tcaggccgtc atcccaggac tccgtctccg ctggtgagaa
gcccccgagg ctctatgatc gcaggagcat cttcgatgct
gtggctcaga gtaactgcca ggagctggag agcctgctgc
ccttcctgca gaggagcaag aagcgcctga ctgacagcga
gttcaaagac ccagagacag gaaagacctg tctgctaaaa
gccatgctca atctgcacaa tgggcagaat gacaccatcg
ctctgctcct ggacgttgcc cggaagacag acagcctgaa
gcagtttgtc aatgccagct acacagacag ctactacaag
ggccagacag cactgcacat tgccattgaa cggcggaaca
tgacgctggt gaccctcttg gtggagaatg gagcagatgt
ccaggctgcg gctaacgggg acttcttcaa gaaaaccaaa
gggaggcctg gcttctactt tggtgagctg cccctgtccc
tggctgcgtg caccaaccag ctggccattg tgaagttcct
gctgcagaac tcctggcagc ctgcagacat cagcgcccgg
gactcagtgg gcaacacggt gcttcatgcc ctggtggagg
tggcagataa cacagttgac aacaccaagt tcgtgacaag
catgtacaac gagatcttga tcctgggggc caaactccac
cccacgctga agctggaaga gatcaccaac aggaaggggc
tcacgccact ggctctggct gctagcagtg ggaagatcgg
ggtcttggcc tacattctcc agagggagat ccatgaaccc
gagtgccgac acctatccag gaagttcacc gaatgggcct
atgggccagt gcactcctcc ctttatgacc tgtcctgcat
tgacacctgt gaaaagaact cggttctgga ggtgatcgct
tacagcagca gtgagacccc taaccgtcat gacatgcttc
tcgtggaacc cttgaaccga ctcctacagg acaagtggga
cagatttgtc aagcgcatct tctacttcaa cttcttcgtc
tactgcttgt atatgatcat cttcaccgcg gctgcctact
atcggcctgt ggaaggcttg cccccctata agctgaaaaa
caccgttggg gactatttcc gagtcaccgg agagatcttg
tctgtgtcag gaggagtcta cttcttcttc cgagggattc
aatatttcct gcagaggcga ccatccctca agagtttgtt
tgtggacagc tacagtgaga tacttttctt tgtacagtcg
ctgttcatgc tggtgtctgt ggtactgtac ttcagccaac
gcaaggagta tgtggcttcc atggtgttct ccctggccat
gggctggacc aacatgctct actatacccg aggattccag
cagatgggca tctatgctgt catgattgag aagatgatcc
tcagagacct gtgccggttt atgttcgtct acctcgtgtt
cttgtttgga ttttccacag ctgtggtgac actgattgag
gatgggaaga ataactctct gcctatggag tccacaccac
acaagtgccg ggggtctgcc tgcaagccag gtaactctta
caacagcctg tattccacat gtctggagct gttcaagttc
accatcggca tgggcgacct ggagttcact gagaactacg
acttcaaggc tgtcttcatc atcctgttac tggcctatgt
gattctcacc tacatccttc tgctcaacat gctcattgct
ctcatgggtg agaccgtcaa caagattgca caagagagca
agaacatctg gaagctgcag agagccatca ccatcctgga
tacagagaag agcttcctga agtgcatgag gaaggccttc
cgctctggca agctgctgca ggtggggttc actcctgacg
gcaaggatga ctaccggtgg tgtttcaggg tggacgaggt
aaactggact acctggaaca ccaatgtggg tatcatcaac
gaggacccag gcaactgtga gggcgtcaag cgcaccctga
gcttctccct gaggtcaggc cgagtttcag ggagaaactg
gaagaacttt gccctggttc cccttctgag ggatgcaagc
actcgagata gacatgccac ccagcaggaa gaagttcaac
tgaagcatta tacgggatcc cttaagccag aggatgctga
ggttttcaag gattccatgg tcccagggga gaaataatgg
acactatgca gggatcaatg cggggtcttt gggtggtctg
cttagggaac cagcagggtt gacgttatct gggtccactc
tgtgcctgcc taggcacatt cctaggactt cggcgggcct
gctgtgggaa ctgggaggtg tgtgggaatt gagatgtgta
tccaaccatg atctccaaac atttggcttt caactcttta
tggactttat taaacagagt gaatggcaaa tctctacttg
gacacat

SEQ ID NO: 9

PRT
Artificial
Chemically synthesized peptide antigen to NPFR1
peptide antibodies.
CMTGHHEGGLRSAIT

SEQ ID NO: 10

PRT
Artificial
Chemically synthesized Peptide antigen to NPFR1
peptide antibodies
SSNSVRYLDDRHPLC

SEQ ID NO: 11

DNA
Artificial
Chemically synthesized primer sequence
caccatggaacaacgggctagctta

SEQ ID NO: 12

DNA
Artificial
Chemically synthesized primer sequence
ttctttctcccctgggaccatgga

SEQ ID NO: 13

DNA
Artificial
Chemically synthesized primer sequence
caccatgataatcagcatgaatcaga

SEQ ID NO: 14

DNA
Artificial
Chemically synthesized primer sequence
ttaccgcggcatcagcttggt

We claim:

1. A method of identifying a composition capable of inhibiting a response to a stressor, comprising:
exposing a Drosophila melanogaster organism to a medium comprising a compound that elicits an avoidance response in a wild-type Drosophila organism, wherein the Drosophila organism exhibits an avoidance response to the medium; and
contacting the Drosophila organism with a test compound, wherein a reduction in the avoidance response to the medium in the presence of the test compound as compared to in the absence of the test compound indicates that the test compound modulates response to a stressor in a higher organism.

2. The method of claim 1, wherein the Drosophila organism over-expresses a neuropeptide receptor NPF1.

3. The method of claim 1, wherein the test compound interacts with a Drosophila NPFR1 polypeptide to inhibit activity of a TPRA ion-channel polypeptide.

4. The method of claim 1, wherein the test compound mimics or modulates the activity of a Drosophila neuropeptide F (NPF), and wherein the compound is not Drosophila NPF.

5. The method of claim 1, wherein the Drosophila organism is a post-feeding larval form of Drosophila, and wherein the compound in the medium that elicits an avoidance response is a sugar.

6. The method of claim 5, wherein the sugar is fructose.

7. The method of claim 1, wherein the Drosophila organism is an adult fly, and wherein the compound in the medium that elicits an avoidance response is isothiocyanate.

8. A method of identifying a composition capable of modulating a response to a stressor, comprising:
providing a recombinant Drosophila melanogaster organism comprising a heterologous transient receptor potential (TRP) ion-channel polypeptide from a different organism, wherein the TRP ion-channel polypeptide is responsive to a stressor;
exposing the larva to the stressor, wherein the Drosophila larva exhibits an avoidance response to the stressor; and
contacting the larva with a test compound, wherein a reduction in the avoidance response to the stressor in the presence of the test compound as compared to in the absence of the test compound indicates that the test compound modulates response to a stressor in a higher organism.

9. The method of claim 8, wherein the TRP ion-channel polypeptide is a rat TRPV1 comprising SEQ. ID NO: 7.

10. The method of claim 9, wherein the stressor is capsaicin.

11. A recombinant Drosophila melanogaster organism comprising: a heterologous transient receptor potential (TRP) ion-channel polypeptide from a different organism, wherein the TRP ion-channel polypeptide is responsive to a stressor.

12. The recombinant Drosophila melanogaster organism of claim 11, wherein the TRP ion-channel polypeptide is a rat TRPV1 and the stressor is capsaicin.

13. A method of identifying a composition capable of inhibiting a response to a stressor, comprising:
providing a recombinant Drosophila melanogaster organism comprising neurons comprising a nucleic acid encoding for a transient receptor potential (TRP) ion-channel polypeptide that is responsive to a stressor, wherein the neurons further comprise a heterologous nucleic acid encoding a neuropeptide family receptor;
exposing the organism to the stressor and observing the organism's response to the stressor;
exposing the organism to the stressor in the presence of a test compound; and
observing the organism's response to the stressor, wherein a change in the organism's response to the stressor in the presence of the test compound as compared to the response in the absence of the test compound indicates that the test compound modulates the response to the stressor.

14. The method of claim 13, wherein the TRP ion-channel polypeptide is a Drosophila TRPA peptide sensitive to noxious heat stimulus, and wherein the stressor is a heat probe.

15. The method of claim 13, wherein the neuropeptide family receptor comprises a neuropeptide receptor from Drosophila.

16. The method of claim 15, wherein the neuropeptide family receptor comprises NPFR1.

17. A recombinant Drosophila melanogaster organism comprising a neural cell comprising:
a nucleic acid encoding for a transient receptor potential (TRP) ion-channel polypeptide that is responsive to a particular stressor, and
a heterologous nucleic acid encoding a neuropeptide family receptor that is not present in the neural cell of a wild-type Drosophila organism.

18. A method of identifying a composition capable of reducing a cellular response to a stressor, comprising:
exposing a first population of cells to a stressor, wherein the cells comprise a heterologous nucleic acid encoding a transient receptor potential (TRP) ion-channel polypeptide and a heterologous nucleic acid encoding a neuropeptide receptor, and a fluorescent compound capable of intracellularly fluorescing in the presence of calcium, wherein the TRP ion-channel polypeptide transports calcium into the cell in response to a stressor;
delivering to a second population of cells a stressor, wherein the cells comprise the heterologous nucleic acid encoding the TRP ion-channel polypeptide, and the heterologous nucleic acid encoding the neuropeptide receptor, the fluorescent compound is capable of intracellularly fluorescing in the presence of calcium, and a test compound; and
determining the difference in the level fluorescence of the first and second cell populations, wherein if the level of fluorescence in the first cell population is greater than in the second cell population, the test compound inhibits the uptake of calcium via the transient receptor potential ion-channel polypeptide.

19. The method of claim 18, wherein the TRP ion-channel polypeptide is Drosophila TRPA.

20. The method of claim 19, wherein the Drosophila TRPA comprises SEQ ID NO: 1.

21. The method of claim 18, wherein, wherein the TRP ion-channel polypeptide is rat TRPV1.

22. The method of claim 21, wherein the rat TRPV1 comprises SEQ ID NO: 7.

23. The method of claim 18, wherein the neuropeptide Y family receptor comprises Drosophila neuropeptide receptor F1 (NPFR1).

24. The method of claim 23, wherein the NPFR1 peptide comprises SEQ ID NO: 3.

25. The method of claim 18, wherein the neuropeptide receptor is a neuropeptide Y family receptor and wherein the test compound interacts with the neuropeptide Y family receptor to inhibit activity of the TPP ion-channel polypeptide.

26. The method of claim 18, wherein the test compound interacts with a Drosophila NPFR1 polypeptide to inhibit activity of a TPRA ion-channel polypeptide.

27. The method of claim 26, wherein the test compound mimics or modulates the activity of a Drosophila neuropeptide F (NPF), and wherein the compound is not Drosophila NPF.

28. A method of identifying a composition capable of reducing a cellular response to a stressor, comprising:
exposing a first population of cells to a stressor, wherein the cells comprise a heterologous nucleic acid encoding a transient receptor potential (TRP) ion-channel polypeptide and a heterologous nucleic acid encoding a neuropeptide receptor, and wherein the cells are in contact with a medium comprising a neuropeptide capable of selectively binding to the neuropeptide receptor, and a fluorescent compound capable of intracellularly fluorescing in the presence of calcium, wherein the TRP ion-channel polypeptide transports calcium into the cell in response to a stressor;
delivering to a second population of cells a stressor, wherein the cells comprise the heterologous nucleic acid encoding the TRP ion-channel polypeptide, and the heterologous nucleic acid encoding the neuropeptide receptor, and wherein the cells are in contact with a medium comprising the neuropeptide capable of selectively binding to the neuropeptide receptor, the fluorescent compound is capable of intracellularly fluorescing in the presence of calcium, and a test compound; and
determining the difference in the level fluorescence of the first and second cell populations, wherein if the level of fluorescence in the first cell population is greater than in the second cell population, the test compound inhibits the uptake of calcium via the transient receptor potential ion-channel polypeptide.

29. The method of claim 28, wherein the TRP ion-channel polypeptide is Drosophila TRPA.

30. The method of claim 29, wherein the Drosophila TRPA comprises SEQ ID NO: 1.

31. The method of claim 28, wherein, wherein the TRP ion-channel polypeptide is rat TRPV1.

32. The method of claim 31, wherein the rat TRPV1 comprises SEQ ID NO: 7.

33. The method of claim 28, wherein the neuropeptide Y family receptor comprises Drosophila neuropeptide receptor F1 (NPFR1).

34. The method of claim 33, wherein the NPFR1 peptide comprises SEQ ID NO: 3.

35. The method of claim 28, wherein the neuropeptide comprises a neuropeptide Y family member

36. The method of claim 28, wherein the neuropeptide comprises neuropeptide F (NPF) from Drosophila melanogaster.

37. The method of claim 36, wherein the NPF comprises SEQ ID NO: 5.

38. A recombinant eukaryotic cell comprising: a heterologous nucleic acid encoding a transient receptor potential (TRP) ion-channel polypeptide, and a heterologous nucleic acid encoding a neuropeptide Y family receptor.

39. The cell of claim 38, wherein the TRP ion-channel polypeptide is Drosophila TRPA.

40. The cell of claim 39, wherein the Drosophila TRPA comprises SEQ ID NO: 1.

41. The cell of claim 38, wherein the TRP ion-channel polypeptide is rat TRPV1.

42. The cell of claim 41, wherein the rat TRPV1 comprises SEQ ID NO: 7

43. The cell of claim 38, wherein the neuropeptide Y family receptor comprises Drosophila neuropeptide receptor F1 (NPFR1).

44. The cell of claim 43, wherein the NPFR1 peptide comprises SEQ ID NO: 3

45. The cell of claim 38, wherein the neuropeptide Y family receptor interacts with a neuropeptide Y family member

46. The cell of claim 45, wherein the neuropeptide comprises neuropeptide F (NPF) from Drosophila melanogaster.


SEQUENCE LISTING (14 sequences)

SEQ ID NO: 1 - protein, 913 aa, Drosophila melanogaster
MDFNNCGFIDPQAQLAGALAKQDIRQFVAALDSGALADLQDDRHTSIYEKALSTPGCRDFIEACIDHGSQVNYINKKLDKAAISYAADSRDPGNLAALLKYRPGNKVQVDRKYGQLTPLNSLAKNLTDENAPDVYSCMQLLLDYGASPNIVDQGEFTPLHHVLRKSKVKAGKKELIQLFLDHPELDIDSYRNGEVRRLLQAQFPELKLPEERHTGPEIDIQTLQRTLRDGDETLFEQQFAEYLQNLKGGADNQLNAHQEEYFGLLQESIKRGRQRAFDVILSTGMDINSRPGRANEANLVETAVIYGNWQALERLLKEPNLRLTPDSKLLNAVIGRLDEPPYDGSSHQRCFELLINSDRVDINEADSGRLVPLFFAVKYRNTSAMQKLLKNGAYIGSKSAFGTLPIKDMPPEVLEEHFDSCITTNGERPGDQNFEIIIDYKNLMRQERDSGLNQLQDEMAPIAFIAESKEMRHLLQHPLISSFLFLKWHRLSVIFYLNFLIYSLFTASIITYTLLKFHESDQRALTAFFGLLSWLGISYLILRECIQWIMSPVRYFWSITNIMEVALITLSIFTCMESSFDKETQRVLAVFTILLVSMEFCLLVGSLPVLSISTHMLMLREVSNSFLKSFTLYSIFVLTFSLCFYILFGKSVEEDQSKSATPCPPLGKKEGKDEEQGFNTFTKPIEAVIKTIVMLTGEFDAGSIQFTSIYTYLIFLLFVIFMTIVLFNLLNGLAVSDTQVIKAQAELNGAICRTNVLSRYEQVLTGHGRAGFLLGNHLFRSICQRLMNIYPNYLSLRQISVLPNDGNKVLIPMSDPFEMRTLKKASFQQLPLSAAVPQKKLLDPPLRLLPCCCSLLTGKCSQMSGRVVKRALEVIDQKNAAEQRRKQEQINDSRLKLIEYKLEQLIQLVQDRK

SEQ ID NO: 2 - DNA, 2977 bp, Drosophila melanogaster
attgatccgcaggcccagctagctggagctttggccaagcaggacatccgacagttcgttgctgccctggacagcggtgccctggccgatctacaagacgaccgccataccagtatctacgagaaggcactctcaacaccaggttgtcgtgacttcattgaagcctgcatcgaccacggcagccaggtgaactacatcaacaagaagctggacaaggccgcaatcagctatgcggctgactctagggatccaggaaacctggcggctctccttaagtaccgccccggaaacaaagtccaggttgatagaaaatatgggcagcttactccacttaactcacttgccaagaatctcacggatgaaaatgccccagacgtgtactcctgcatgcaactcttgctggactacggcgcctcgccgaatatcgtagaccagggcgagttcacacccttgcaccatgtgctgagaaagagcaaggtgaaggctgggaagaaggaactgattcagctctttctggaccatccggagctggatatcgatagttaccgaaacggggaggtgcgcagactgctgcaggcgcaatttccggagcttaagctgccggaagagcgtcataccgggccggagattgacatccaaactcttcaaaggactctacgggacggggacgaaacactgtttgagcagcagttcgctgagtacttgcagaatctcaaaggcggagcggataaccaactaaatgcccaccaggaggaatacttcggactgctgcaggagagcatcaagaggggcaggcagcgagccttcgatgtcattttgtccactggcatggatatcaactcgagaccaggcagggccaacgaggccaatctcgtagagacggccgtgatatacggtaactggcaggcgttggagcgactgcttaaggagccaaacctgcgacttactccagactccaagctactaaatgcagtaatcggccgtctggatgagccaccgtatgatggctccagccaccagcgctgctttgaattgctcattaacagcgatcgcgtagacatcaacgaagctgattccggacgcctggtgcctctgttcttcgctgttaagtaccgcaacacgagtgcgatgcaaaaactcctgaagaacggtgcctacattggttctaagagcgcatttggcacactacccatcaaggacatgccacccgaggttctcgaagagcacttcgactcgtgtatcaccacaaacggagagaggcctggtgaccagaactttgagatcatcatcgattataagaacctaatgcgccaggagagagactccggactcaaccagctgcaagacgaaatggccccgatcgcattcatcgccgagtcgaaggagatgcgccacctgctccagcacccgctgatctcgagctttctattcctcaagtggcaccgactttccgtgatattctacctgaacttcctgatatactcgctttttaccgcctccataattacctacacgctcctcaagttccacgaaagcgatcaaagggctcttactgcatttttcggattgctttcctggctgggaatcagctaccttatattacgggagtgcatccagtggataatgtctccagttcggtacttttggtctataacgaatattatggaggtggctcttattacactatctatctttacctgcatggaatccagcttcgacaaggagacgcagcgcgtcttagccgtatttaccatcctactcgtctccatggagttttgtttactagtgggctccctgccagtgctctcaatttcgacgcacatgctgatgctgcgagaggtgtcaaacagcttcttaaagagctttaccctctactcgatcttcgtgctcaccttcagcctgtgtttctatatcctcttcggcaagtcagtggaggaagaccagtctaaaagcgctacgccatgtccacctctggggaagaaggaggggaaggacgaggaacagggcttcaacacatttaccaagcctatcgaggccgtgatcaagaccattgtgatgctgacaggcgagtttgacgccggaagcatccagtttaccagcatctacacctacctgattttcctgctcttcgtgatctttatgacgatagtgctgttcaaccttttgaacggtcttgcagtgagcgacacccaagttattaaggctcaggcggaactgaacggagccatttgcagaaccaacgtccttagtcggtacgagcaggttctcactggccacggacgcgctgggtttttgttgggcaaccatctcttccgcagcatctgccaacgtttgatgaacatctacccgaactacttaagtctgcgtcagatttccgtgctgccgaacgatggaaacaaagtgcttattccaatgagcgatcccttcgaaatgaggacccttaagaaggctagctttcagcaattgcccctgagtgctgcagtgccccagaagaagctgttggatccaccgcttagacttctgccctgctgctgttccctgctcaccggaaagtgctcccagatgagcggccgggtggtcaaacgggccctcgaggtaatcgatcagaagaacgcggcggagcagaggcggaaacaggaacagatcaacgacagtcgactgaagctgatcgagtacaagctggagcaattaatacagctggtccaggaccggaagtgatggagaatgtattttggtagctttagtatttatgagactaatcaaccttttagaacgatttgcatttaacattcagtttaaagagccgagttagtcggaaattgtttttattaacatacgagtaatgaaattgaacaaaacccttaataattgtcagtaagtaagtagtatataatggttatatagacagtaaatattgtataaacgaatatcattactgtactatttgtacccgagtaaatatttaatttcaaatgtt

SEQ ID NO: 3 - protein, 485 aa, Drosophila melanogaster
MIISMNQTEPAQLADGEHLSGYASSSNSVRYLDDRHPLDYLDLGTVHALNTTAINTSDLNETGSRPLDPVLIDRFLSNRAVDSPWYHMLISMYGVLIVFGALGNTLVVIAVIRKPIMRTARNLFILNLAISDLLLCLVTMPLTLMEILSKYWPYGSCSILCKTIAMLQALCIFVSTISITAIAFDRYQVIVYPTRDSLQFVGAVTILAGIWALALLLASPLFVYKELINTDTPALLQQIGLQDTIPYCIEDWPSRNGRFYYSIFSLCVQYLVPILIVSVAYFGIYNKLKSRITVVAVQASSAQRKVERGRRMKRTNCLLISIAIIFGVSWLPLNFFNLYADMERSPVTQSMLVRYAICHMIGMSSACSNPLLYGWLNDNFRKEFQELLCRCSDTNVALNGHTTGCNVQAAARRRRKLGAELSKGELKLLGPGGAQSGTAGGEGGLAATDFMTGHHEGGLRSAITESVALTDHNPVPSEVTKLMPR

SEQ ID NO: 4 - DNA, 3140 bp, Drosophila melanogaster
aacagatggtcgctgactgtgcacgcgtgtggttatcggagatcagtaaacagcccaactaaacaccgaaacttactgtaataaaaaaaaacgggaaataagcgaaataatcaaaatgcggccgcatacttatttataattttgaggcggccgagcaccggggccccaaactctttggatctgcacggaatccagaattccgagagagcaaaaacacaaagcgaagtcccgtgagtgcattccaagttgaaaactaagtgagcaactgctgctttggcagccggaaaaacagagattcactcgtgtcactcgcagaaggaaaaacaagaaccgacggccaggaaaacaatacggtaccacgcactatagtaaatatatagcatacatatccccagggcgaaggagattgccaggacgatgataatcagcatgaatcagacggagcccgcccagctggcagatggggagcatctgagtggatacgccagcagcagcaacagcgtgcgctatctggacgaccggcatccgctggactaccttgacctgggcacggtgcacgccctcaacaccactgccatcaacacctcggatctgaatgagactgggagcaggccgctggacccggtgcttatcgataggttcctgagcaacagggcggtggacagcccctggtaccacatgctcatcagcatgtacggcgtgctaatcgtcttcggcgccctaggcaacaccctggttgttatagccgtcatccggaagcccatcatgcgcactgctcgcaatctgttcatcctcaacctggccatatcggacctacttttatgcctagtcaccatgccgctgaccttgatggagatcctgtccaagtactggccctacggctcctgctccatcctgtgcaaaacgattgccatgctgcaggcactttgtattttcgtgtcgacaatatccataacggccattgccttcgacagatatcaggtgatcgtgtaccccacgcgggacagcctgcagttcgtgggcgcggtgacgatcctggcggggatctgggcactggcactgctgctggcctcgccgctgttcgtctacaaggagctgatcaacacagacacgccggcactcctgcagcagatcggcctgcaggacacgatcccgtactgcattgaggactggccaagtcgcaacgggcgcttctactactcgatcttctcgctgtgcgtacaatacctggtgcccatcctgatcgtctcggtggcatacttcgggatctacaacaagctgaagagccgcatcaccgtggtggctgtgcaggcgtcctccgctcagcggaaggtggagcgggggcggcggatgaagcgcaccaactgcctactgatcagcatcgccatcatctttggcgtttcttggctgccgctgaactttttcaacctgtacgcggacatggagcgctcgccggtcactcagagcatgctagtccgctacgccatctgccacatgatcggcatgagctccgcctgctccaacccgttgctctacggctggctcaacgacaacttccgtaaagaatttcaagaactgctctgccgttgctcagacactaatgttgctcttaacggtcacacgacaggctgcaacgtccaggcggcggcgcgcaggcgtcgcaagttgggcgccgaactctccaaaggcgaactcaagctgctggggccaggcggcgcccagagcggtaccgccggcggggaaggcggtctggcggccaccgacttcatgaccggccaccacgagggcggactgcgcagcgccataaccgagtcggtggccctcacggaccacaaccccgtgccctcggaggtcaccaagctgatgccgcggtaaagcacagggtagtcctaaggtccttgaggtctggtctcgtgtctaagtcctcatgatacacgcgtgcatgtccttttgtacgccctcgggctgattggatttgcatgctccaaacgtcgctgctgctcgctttacgtttcacttgttccaactgcaactgccacctctctagaacactgagcgaaatgccgtgtcctctaatcgggaaacactctggctgtaaaatctataagcagccgagtcaaacgtttctagcgttctaaaagtttcttatgattattttattttatatattaataacaatgacttcgttcccaattatatgcttgttttcatcgtttttgaatgtaacaattgatcaatattcgaccaaaagcaagttttgaaatatttgtgtaaatatcgttttcaaatttgttcgccttaatcttacttaataataataataataataatctttactccgataatcattaacgtaacatttctacttgtaaaaatatttctgatctaaggggcttgctcttttcggctccaccttgaattacttttcagttgactaactaggcgtatatttttgtcagtgtatgcatgcgctcctcttatcgttgccttgtcgagctgtaactcttgttgttgccatgttgtgacattatttgcttttgagctgcaatcgattatgacccgtcttttgtgacacattttcagtttggaacagtactaaattggcaatcaatcctggagcagcaggcggctggagcagcattctagggaggcgactgcctgtcacccatagacttaacacgtattgtccagtgatccaaagccaagctgagttagccctaagttaagacacacgcgactaagagctcgaagcctgtaaactattcttaaacgaccatgtcatggcatccatcatcaactcggactaagtctatggttagatcactttcgtatcaaatgccgaaaagtaattgaatgagcccccaattagatttcgggtatttgataagtcgaatacgcctaagactcgaataagttctctcaacagttcctaggaaatattttcgttttatctcagcatttcttggtatcatctaagctaaggattagctataattgatgttctgttcgctattcaataatgataggataggtcgaagtccactaagccaaagtgaatttaaagtatagtatagtataaagtataattgtaaaagataactttaaaataaatgtcagctgctcttaaacatttaatgcaa

SEQ ID NO: 5 - protein, 102 aa, Drosophila melanogaster
MCQTMRCILVACVALALLAAGCRVEASNSRPPRKNDVNTMADAYKFLQDLDTYYGDRARVRFGKRGSLMDILRNHEMDNINLGKNANNGGEFARGFNEEEIF

SEQ ID NO: 6 - DNA, 570 bp, Drosophila melanogaster
tacagtccgacgaacaattgcattgtgacaccgttgcgctttccaatactcaaactcccagttgaaccagaactatgtgccaaacaatgcgttgcatcctggttgcctgtgtggcccttgccctcctagccgccggctgccgagtggaggcgtccaactccagacctccgcgaaagaacgatgtcaacactatggctgatgcctacaagttcctgcaggatctggacacctactacggcgacagagcccgcgttcggttcggaaagcgcggatcgctgatggatatcctgaggaatcacgagatggacaacataaatctaggaaaaaatgccaacaatggaggagaatttgctcgcggttttaatgaggaggagatattctaaatccattttagacgaccatggcaacgtcactaactcatgatgatagttattagcatacgcatttttatttaaattgtttttcggggcaatagtttaacgtgctgggaaagaacaagtagttgcagctacagaaataagtatttactctagtcttgatgtcggttgaataaatgaattaccccaataa

SEQ ID NO: 7 - protein, 838 aa, Rattus norvegicus
MEQRASLDSEESESPPQENSCLDPPDRDPNCKPPPVKPHIFTTRSRTRLFGKGDSEEASPLDCPYEEGGLASCPIITVSSVLTIQRPGDGPASVRPSSQDSVSAGEKPPRLYDRRSIFDAVAQSNCQELESLLPFLQRSKKRLTDSEFKDPETGKTCLLKAMLNLHNGQNDTIALLLDVARKTDSLKQFVNASYTDSYYKGQTALHIAIERRNMTLVTLLVENGADVQAAANGDFFKKTKGRPGFYFGELPLSLAACTNQLAIVKFLLQNSWQPADISARDSVGNTVLHALVEVADNTVDNTKFVTSMYNEILILGAKLHPTLKLEEITNRKGLTPLALAASSGKIGVLAYILQREIHEPECRHLSRKFTEWAYGPVHSSLYDLSCIDTCEKNSVLEVIAYSSSETPNRHDMLLVEPLNRLLQDKWDRFVKRIFYFNFFVYCLYMIIFTAAAYYRPVEGLPPYKLKNTVGDYFRVTGEILSVSGGVYFFFRGIQYFLQRRPSLKSLFVDSYSEILFFVQSLFMLVSVVLYFSQRKEYVASMVFSLAMGWTNMLYYTRGFQQMGIYAVMIEKMILRDLCRFMFVYLVFLFGFSTAVVTLIEDGKNNSLPMESTPHKCRGSACKPGNSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGFTPDGKDDYRWCFRVDEVNWTTWNTNVGIINEDPGNCEGVKRTLSFSLRSGRVSGRNWKNFALVPLLRDASTRDRHATQQEEVQLKHYTGSLKPEDAEVFKDSMVPGEK

SEQ ID NO: 8 - DNA, 2847 bp, Rattus norvegicus
cagctccaaggcacttgctccatttggggtgtgcctgcacctagctggttgcaaattgggccacagaggatctggaaaggatggaacaacgggctagcttagactcagaggagtctgagtccccaccccaagagaactcctgcctggaccctccagacagagaccctaactgcaagccacctccagtcaagccccacatcttcactaccaggagtcgtacccggctttttgggaagggtgactcggaggaggcctctcccctggactgcccttatgaggaaggcgggctggcttcctgccctatcatcactgtcagctctgttctaactatccagaggcctggggatggacctgccagtgtcaggccgtcatcccaggactccgtctccgctggtgagaagcccccgaggctctatgatcgcaggagcatcttcgatgctgtggctcagagtaactgccaggagctggagagcctgctgcccttcctgcagaggagcaagaagcgcctgactgacagcgagttcaaagacccagagacaggaaagacctgtctgctaaaagccatgctcaatctgcacaatgggcagaatgacaccatcgctctgctcctggacgttgcccggaagacagacagcctgaagcagtttgtcaatgccagctacacagacagctactacaagggccagacagcactgcacattgccattgaacggcggaacatgacgctggtgaccctcttggtggagaatggagcagatgtccaggctgcggctaacggggacttcttcaagaaaaccaaagggaggcctggcttctactttggtgagctgcccctgtccctggctgcgtgcaccaaccagctggccattgtgaagttcctgctgcagaactcctggcagcctgcagacatcagcgcccgggactcagtgggcaacacggtgcttcatgccctggtggaggtggcagataacacagttgacaacaccaagttcgtgacaagcatgtacaacgagatcttgatcctgggggccaaactccaccccacgctgaagctggaagagatcaccaacaggaaggggctcacgccactggctctggctgctagcagtgggaagatcggggtcttggcctacattctccagagggagatccatgaacccgagtgccgacacctatccaggaagttcaccgaatgggcctatgggccagtgcactcctccctttatgacctgtcctgcattgacacctgtgaaaagaactcggttctggaggtgatcgcttacagcagcagtgagacccctaaccgtcatgacatgcttctcgtggaacccttgaaccgactcctacaggacaagtgggacagatttgtcaagcgcatcttctacttcaacttcttcgtctactgcttgtatatgatcatcttcaccgcggctgcctactatcggcctgtggaaggcttgcccccctataagctgaaaaacaccgttggggactatttccgagtcaccggagagatcttgtctgtgtcaggaggagtctacttcttcttccgagggattcaatatttcctgcagaggcgaccatccctcaagagtttgtttgtggacagctacagtgagatacttttctttgtacagtcgctgttcatgctggtgtctgtggtactgtacttcagccaacgcaaggagtatgtggcttccatggtgttctccctggccatgggctggaccaacatgctctactatacccgaggattccagcagatgggcatctatgctgtcatgattgagaagatgatcctcagagacctgtgccggtttatgttcgtctacctcgtgttcttgtttggattttccacagctgtggtgacactgattgaggatgggaagaataactctctgcctatggagtccacaccacacaagtgccgggggtctgcctgcaagccaggtaactcttacaacagcctgtattccacatgtctggagctgttcaagttcaccatcggcatgggcgacctggagttcactgagaactacgacttcaaggctgtcttcatcatcctgttactggcctatgtgattctcacctacatccttctgctcaacatgctcattgctctcatgggtgagaccgtcaacaagattgcacaagagagcaagaacatctggaagctgcagagagccatcaccatcctggatacagagaagagcttcctgaagtgcatgaggaaggccttccgctctggcaagctgctgcaggtggggttcactcctgacggcaaggatgactaccggtggtgtttcagggtggacgaggtaaactggactacctggaacaccaatgtgggtatcatcaacgaggacccaggcaactgtgagggcgtcaagcgcaccctgagcttctccctgaggtcaggccgagtttcagggagaaactggaagaactttgccctggttccccttctgagggatgcaagcactcgagatagacatgccacccagcaggaagaagttcaactgaagcattatacgggatcccttaagccagaggatgctgaggttttcaaggattccatggtcccaggggagaaataatggacactatgcagggatcaatgcggggtctttgggtggtctgcttagggaaccagcagggttgacgttatctgggtccactctgtgcctgcctaggcacattcctaggacttcggcgggcctgctgtgggaactgggaggtgtgtgggaattgagatgtgtatccaaccatgatctccaaacatttggctttcaactctttatggactttattaaacagagtgaatggcaaatctctacttggacacat

SEQ ID NO: 9 - protein, 15 aa, Artificial Sequence
  Chemically synthesized peptide antigen to NPFR1 peptide antibodies
CMTGHHEGGLRSAIT

SEQ ID NO: 10 - protein, 15 aa, Artificial Sequence
  Chemically synthesized peptide antigen to NPFR1 peptide antibodies
SSNSVRYLDDRHPLC

SEQ ID NO: 11 - DNA, 25 bp, Artificial Sequence
  Chemically synthesized primer sequence
caccatggaacaacgggctagctta

SEQ ID NO: 12 - DNA, 24 bp, Artificial Sequence
  Chemically synthesized primer sequence
ttctttctcccctgggaccatgga

SEQ ID NO: 13 - DNA, 26 bp, Artificial Sequence
  Chemically synthesized primer sequence
caccatgataatcagcatgaatcaga

SEQ ID NO: 14 - DNA, 21 bp, Artificial Sequence
  Chemically synthesized primer sequence
ttaccgcggcatcagcttggt